(19) United States
(12) Patent Application Publication
(10) Pub. No.: US 2024/0361289 A1
(43) Pub. Date: Oct. 31, 2024

(54) SYSTEMS AND METHODS FOR ANALYZING AIR OF AN INDOOR ENVIRONMENT

(71) Applicant: OpenAeros LLC, St.Paul, MN (US)

(72) Inventors: Aaron Collins, St. Paul, MN (US); Chia Hua M. Pang, St. Paul, MN (US); Rachel Andreasen, St. Paul, MN (US); Christopher Richard Saline Libby, St. Louis Park, MN (US); Louis Goessling, Minneapolis, MN (US)

(21) Appl. No.: 18/648,179

(22) Filed: Apr. 26, 2024

Related U.S. Application Data
(60) Provisional application No. 63/462,099, filed on Apr. 26, 2023.

(51) Int. Cl. G01N 33/00; F24F 11/39; F24F 110/64; F24F 110/66; F24F 110/70; F24F 120/10

(52) U.S. Cl. CPC G01N 33/0067; F24F 11/39; F24F 2110/64; F24F 2110/66; F24F 2110/70; F24F 2120/10

(57) ABSTRACT

Systems and methods for analysing air of an indoor environment having a plurality of particle mitigation systems. The system can comprise a particle monitor configured to receive air from the indoor environment to monitor particles, a gas monitor configured to receive air from the indoor environment to monitor a gas, and a control system operatively coupled to the particle monitor and the gas monitor to determine at least one air characteristic, which may include one or more of clean air delivery rate (CADR) for the indoor environment, room air volume, air change rate (ACH) of ventilation for the indoor environment, and mitigation equivalent air change rate (MEACH) for the plurality of particle mitigation systems, or portion thereof.

### CROSS-REFERENCE TO RELATED APPLICATION

This application claims the benefit of priority to U.S. Provisional Patent Application Ser. No. 63/462,099, filed Apr. 26, 2023, the disclosure of which is incorporated herein in its entirety by reference.

### TECHNICAL FIELD

The present disclosure is related to systems and methods for analyzing air of an indoor environment, including indoor environments having mechanical filtration.

### BACKGROUND

Individuals spend large amounts of time in indoor environments while working, learning, recreating, and sleeping. These indoor environments often include air handling systems, which can be configured to bring in fresh air from the outside environment, move air through air cleaning systems to mitigate particles, and recirculate air within the indoor environment. Air within an indoor environment contains a variety of suspended particles often called aerosols. Common sources of particles include combustion sources, cooking, cleaning (e.g., vacuuming, sweeping, dusting, use of cleaning agents, and more), occupants tracking debris from outdoors to indoors, introduction of air pollutants to indoor environments via openings, occupant and pet dander, proximity to transit, machine work, mechanical abrasion, sanding, and grinding. Inhabitants and other occupants of rooms can be significant sources of particles, particularly infectious particles. If infected with a disease in which airborne transmission is a vector, the infected occupant can exhale infectious particles into the space. These infectious and other particles may present a health risk to other occupants of the indoor environment.

Many indoor environments include particle mitigation systems to lower the number of particles in an environment which remove or otherwise eliminate various particles from the air. An exemplary particle mitigation system is a mechanical filtration system (e.g. systems utilizing MERV or HEPA rated filters), which removes particles at various rates—depending on particle size—from the air within the indoor environment. Mechanical filtration systems may be stand-alone or portable units positioned within the indoor environment or they may be integrated with an air handling system as part of the recirculation air flow path of an air handling system. Air handling systems can also serve as particle mitigation systems commonly providing access to both ventilation and mechanical filtration. Another exemplary particle mitigation system is a light source to which the target particle is sensitive and which results in destruction of the target particle, such as germicidal ultraviolet radiation. A further exemplary particle mitigation system is an electrostatic system, which ionizes the target particle, enhancing particle collection through electrostatic forces.

It can be difficult to assess the air quality of indoor spaces and even more difficult to determine the efficacy or performance of air handling and particle mitigation systems that are installed to address air quality.

There remains a need for improved systems and methods for analyzing the air of an indoor environment and for analyzing the effect of particle mitigation systems therein.

### SUMMARY

In embodiments of the present disclosure, an air characterization system is provided which may determine one or more characteristics related to at least one particle mitigation system in an indoor environment. In embodiments of the present disclosure, an air characterization system is provided which may determine one or more characteristics related to a plurality of particle mitigation systems in an indoor environment. In embodiments of the present disclosure, an air characterization system may provide feedback to an operator regarding the effectiveness of the particle mitigation systems in an indoor environment.

In an exemplary embodiment of the present disclosure, a system for analyzing air of an indoor environment having a plurality of particle mitigation systems including at least a mechanical air filtration system having a particle filtration profile wherein a first particle size is filtered less than a second particle size and an air handling system for exchanging air with an outdoor environment is provided. The system comprising a particle generator configured to charge the indoor environment with a first charge including a concentration of a plurality of test particles; a particle monitor configured to receive air from the indoor environment to detect the plurality of test particles; a gas generator configured to charge the indoor environment with a second charge including a concentration of a test gas; a gas monitor configured to receive air from the indoor environment to detect the test gas; and a control system operatively coupled to the particle monitor and the gas monitor to determine at least one air characteristic of the indoor environment based on a test gas characteristic related to a first portion of the particle mitigation systems of the indoor environment and a test particle characteristic related to a second portion of the particle mitigation systems of the indoor environment. The plurality of test particles having a test particle size about equal to the first particle size. The test gas having a size less than the test particle size.

In another exemplary embodiment of the present disclosure, a system for analyzing air of an indoor environment having a plurality of particle mitigation systems including at least a mechanical air filtration system having a particle filtration profile wherein a first particle size is filtered less than a second particle size, an air handling system for exchanging air with an outdoor environment, and a light system is provided. The system comprising a first monitor operable to characterize the air handling system; a second monitor operable to characterize the mechanical air filtration system; a third monitor operable to characterize the light system; and a control system operatively coupled to the first monitor, the second monitor, and the third monitor to determine at least one air characteristic of the indoor environment.

In a further exemplary embodiment of the present disclosure, a system for analyzing air of an indoor environment having at least one particle mitigation system is provided. The at least one particle mitigation system receiving air from an outdoor environment and filtering air which has been circulated in the indoor environments with at least one filter having a particle filtration profile wherein a first particle size is filtered less than a second particle size. The system comprising a particle generator configured to charge the indoor environment with a first charge including a concentration of a plurality of test particles; a particle monitor configured to receive air from the indoor environment to detect the test particle during a first time window; a gas generator configured to charge the indoor environment with a second charge including a concentration of a test gas; a gas monitor configured to receive air from the indoor environment to detect the test gas during the first time window; and a control system operatively coupled to the particle monitor and the gas monitor to determine at least one air characteristic of the indoor environment. The plurality of test particles having a test particle size about equal to the first particle size. The test gas having a size less than the test particle size.

In another exemplary embodiment of the present disclosure, a system for analyzing air of an indoor environment having a plurality of particle mitigation systems. The system includes a particle monitor configured to receive air having particles generated by a particle generator in the indoor environment for monitoring a particle characteristic over time. The system includes a gas monitor configured to receive air having a gas generated by a gas generator in the indoor environment and monitor a gas characteristic over time. The system includes a control system operatively coupled to the particle monitor and the gas monitor to determine at least one air characteristic of the indoor environment based on both the particle characteristic and the gas characteristic.

In yet another exemplary embodiment of the present disclosure, a system for analyzing air of an indoor environment having a plurality of particle mitigation systems. The system includes a data input configured to receive a mitigation equivalent air change rate (MEACH) for the plurality of particle mitigation systems, or one or more portion thereof. The system includes a gas monitor configured to receive air having a gas generated by a gas generator in the indoor environment and monitor a gas characteristic over time. The system includes a control system operatively coupled to the gas monitor to determine at least one air characteristic of the indoor environment based on both the gas characteristic and the mitigation equivalent air change rate (MEACH).

In a further exemplary embodiment of the present disclosure, a system for analyzing air of an indoor environment having a plurality of particle mitigation systems. The system includes a particle monitor configured to receive air having particles generated by occupant activity in the indoor environment and monitor a particle characteristic over time. The system includes a gas monitor configured to receive air having a gas generated by occupants in the indoor environment and monitor a gas characteristic over time. The system includes a control system operatively coupled to the particle monitor and the gas monitor to determine at least one air characteristic of the indoor environment based on both the particle characteristic and the gas characteristic.

In another exemplary embodiment of the present disclosure, a method for analyzing air of an indoor environment having a plurality of particle mitigation systems. The method involves: monitoring over time a particle characteristic of air having particles generated by a particle generator in the indoor environment; monitoring over time a gas characteristic of air having gas generated by a gas generator in the indoor environment; and determining at least one air characteristic of the indoor environment based on both the particle characteristic and the gas characteristic.

In another exemplary embodiment of the present disclosure, a method for analyzing air of an indoor environment having a plurality of particle mitigation systems, the method comprising: providing a mitigation equivalent air change rate (MEACH) for the plurality of particle mitigation systems, or one or more portion thereof; monitoring over time a gas characteristic of air having gas generated by a gas generator in the indoor environment; determining at least one air characteristic of the indoor environment based on both the gas characteristic and the mitigation equivalent air change rate (MEACH).

In yet another exemplary embodiment of the present disclosure, a method for analyzing air of an indoor environment having a plurality of particle mitigation systems, the method comprising: monitoring over time a particle characteristic of air having particles generated by occupant activity in the indoor environment; monitoring over time a gas characteristic of air having a gas generated by occupants in the indoor environment; and determining at least one air characteristic of the indoor environment based on both the particle characteristic and the gas characteristic.

Other aspects and embodiments will become apparent from the following description provided below with reference to the accompanying drawings.

Various aspects of the presently described system and method can have one or more advantages related to monitoring air quality and particle mitigation systems in a given space, such as (i) more accurately determining air quality in an indoor environment in the context of known and unknown sources of air exchange with external spaces; (ii) more accurately determining the contribution and performance of particle mitigation systems, and discrete components thereof, in light of known and unknown sources of air exchange with external spaces; (iii) improved identification of which settings, mitigation techniques, or portions of particle mitigation systems can be modified to improve air quality in a given space; (iv) the ability to determine air volume of an indoor environment, or room size, without requiring measurement of physical dimensions; (v) the ability to monitor particles and gas in a given room to assess air quality, ventilation, and particle mitigation efforts in an ad hoc or passive manner without requiring manual tracer testing; (vi) the ability to identify when settings of a particle mitigation system, mechanical filtration system, air handler, fan system, scrubber, germicidal light system, or ventilation should be adjusted based on monitoring gas together with a known or predetermined mitigation equivalent air change rate (MEACH) for the particle mitigation systems, without requiring additional monitoring of particles; (vii) providing accelerated, near real-time, or real-time analysis of air quality and particle mitigation systems in a given space by utilizing different schedules for gas monitoring versus particle monitoring, which likewise can provide more responsive adjustment of settings of a particle mitigation system, mechanical filtration system, air handler, fan system, scrubber, germicidal light system, or ventilation; (viii) the ability to determine air quality in an indoor environment, determine contribution or performance of particle mitigation systems or portion thereof, ventilation in an indoor environment, or room volume of a space, by monitoring occupancy and occupant activity to utilize occupants as gas generators so as not to require a device-based gas generator; (ix) the ability to determine air quality in an indoor environment, determine contribution or performance of particle mitigation systems or portion thereof, ventilation in an indoor environment, or room volume of a space, by monitoring occupancy and occupant activity to utilize occupants as particle generators so as not to require a device-based particle generator; and (x) the ability of portable, transient, or temporary assessment of air quality, ventilation, and/or particle mitigation in an indoor environment even for occupied or in-use spaces, such as busy offices, classrooms, schools, restaurants, kitchens, shopping outlets, health care environments, operating rooms, public transport spaces, buses, trains, airports, airplanes, entertainment venues, and more.

Operative coordination between the gas monitoring and particle monitoring can result in higher resolving power for disambiguating how different portions of the indoor environment contribute to particle mitigation. The amount of ventilation can fluctuate widely, including on fast, short time scales. Ventilation fluctuation can be a significant source of error when particle monitoring. Sources of ventilation include windows, doors, leakage, occupant movement and activities, and air handling, some of which are apparent and predictable and some of which are not. Particle mitigation, in contrast, changes on a more predictable time scale, but its changes can also correlate with ventilation. The higher resolving power of coordinated gas and particle measurements can disambiguate the performance of the particle mitigation systems from overall particle removal, or even can resolve the contribution of individual parts of particle mitigation systems. These advantages can even hold when assessing indoor environments during periods of use, occupant entry or departure, occupant movement, occupant activity, ventilation changes, and other conditions that have been incompatible with particle mitigation assessment. Further, the use of gas monitoring can serve to provide useful information about particle mitigation between particle monitoring periods or during periods when particle monitoring may be difficult, and the resulting gas-based air characteristics can be used to inform adjustment of particle mitigation systems.

Lastly, an indoor space charged with a known or measured amount of gas or particles can permit the room volume to be determined from gas or particle monitoring. This approach avoids the need for physical measurement of the room, it can provide a more accurate volume based on air volume rather than room perimeter, and it can be used even when rooms are occupied, full of furniture or other objects (which displace air volume), in use, or otherwise would be difficult to physically measure. This volume data can be taken a single time or multiple times, and can advantageously be used to improve the accuracy, improve the speed, or improve the convenience of each method, system, and step described herein that involves determining an air characteristic, a particle characteristic, a gas characteristic, particle concentration, particle number, gas concentration, gas number, ACH, MEACH, or CADR values.

### BRIEF DESCRIPTION OF THE DRAWINGS

The above-mentioned and other features and advantages of this disclosure, and the manner of attaining them, will become more apparent and will be better understood by reference to the following description of exemplary embodiments taken in conjunction with the accompanying drawings, wherein:

FIG. 1 illustrates a representative view of an indoor environment including a plurality of exemplary particle mitigation systems and an exemplary air characterization system;

FIG. 2 illustrates a representative view of an exemplary air characterization system;

FIG. 3 illustrates a representative view of further exemplary air characterization system;

FIG. 4 illustrates a representative view of another exemplary air characterization system, which utilizes human occupancy and activity as a gas source for gas generation;

FIG. 5 illustrates a representative view of another exemplary air characterization system, which utilizes a pressurized gas source for gas generation;

FIG. 6 illustrates an exemplary charging curves for the exemplary air characterization system of FIG. 2;

FIG. 7 illustrates an exemplary processing sequence of the exemplary air characterization system of FIG. 2 related to charging the indoor environment with at least one test gas;

FIG. 8 illustrates another exemplary processing sequence of the exemplary air characterization system of FIG. 2 related to charging the indoor environment with at least one test gas;

FIG. 9 illustrates a further exemplary processing sequence of the exemplary air characterization system of FIG. 2 related to charging the indoor environment with at least one test gas;

FIG. 10 illustrates an exemplary processing sequence of the exemplary air characterization system of FIG. 2 related to charging the indoor environment with at least one test particle;

FIG. 11 illustrates an exemplary processing sequence of the exemplary air characterization system of FIG. 2 related to determining an indoor environment air characteristic;

FIG. 12 illustrates another exemplary processing sequence of the exemplary air characterization system of FIG. 2 related to charging the indoor environment with at least one test particle;

FIG. 13 illustrates another exemplary processing sequence of the exemplary air characterization system of FIG. 2 related to determining an indoor environment air characteristic;

FIG. 14 illustrates a further exemplary processing sequence of the exemplary air characterization system of FIG. 2 related to determining an indoor environment air characteristic;

FIG. 15 illustrates an exemplary method of air characterization utilizing a air change rate based on gas removal to determine a mitigation equivalent air change rate (MEACH) for one or more portion of a plurality of particle mitigation systems;

FIG. 16 illustrates an exemplary method of air characterization utilizing particle and gas measurements to determine an air characteristic of the indoor environment;

FIG. 17 illustrates an exemplary method of air characterization utilizing an air change rate based on gas removal of a gas generated by occupants together with a MEACH for a plurality of particle mitigation systems, or one or more portion thereof, for repeatable testing and system adjustment in an indoor environment;

FIG. 18 illustrates an exemplary method of air characterization utilizing an air change rate based on gas removal of a gas generated by occupants together with a separately obtained MEACH for a plurality of particle mitigation systems, or one or more portion thereof, for repeatable testing and system adjustment in an indoor environment;

FIG. 19 illustrates an exemplary method of air characterization utilizing a first gas and a second gas having differing interactions with a portion of a plurality of particle mitigation systems to determine a mitigation equivalent air change rate (MEACH) for said portion of the particle mitigation system, the portion can be a gas-removing portion;

FIG. 20 illustrates an exemplary method of air characterization utilizing particles, a first gas, and a second gas having differing interactions with a portion of a plurality of particle mitigation systems to determine a mitigation equivalent air change rate (MEACH) for said portion of the particle mitigation system;

FIG. 21 illustrates an exemplary method of air characterization utilizing particles, a first gas, and a second gas having differing interactions with a portion of a plurality of particle mitigation systems to assess an air quality criteria for repeatable testing and system adjustment in an indoor environment;

FIG. 22 illustrates an exemplary method of air characterization utilizing a first gas (isopropanol) and a second gas (CO2) generated by occupants, together with a MEACH for a plurality of particle mitigation systems, or one or more portion thereof, for repeatable testing and system adjustment in an indoor environment via CO2 monitoring;

FIG. 23 illustrates an exemplary method of air characterization utilizing occupant monitoring, CO2 monitoring, and particle monitoring, to determine a mitigation equivalent air change rate (MEACH) for a plurality of particle mitigation systems, or one or more portions thereof;

FIG. 24 illustrates an exemplary method of air characterization utilizing occupant monitoring, a first gas (CO2) monitoring, a second gas (isopropanol) monitoring, and particle monitoring, to determine a mitigation equivalent air change rate (MEACH) for portions of a plurality of particle mitigation systems such as mechanical filtration, scrubber, or electrostatic systems;

FIG. 25A and FIG. 25B provide example charts illustrating how to determine removal rate based on recording gas or particle characteristics in air of an indoor space;

FIG. 26 provides example data illustrating how to determine removal rate based on recording gas or particle characteristics in air of an indoor space;

FIG. 27 illustrates an exemplary test gas charging system in a first configuration;

FIG. 28 illustrates the test gas charging system of FIG. 27 in a second configuration;

FIG. 29 illustrates another exemplary test gas charging system in a first configuration;

FIG. 30 illustrates the test gas charging system of FIG. 29 in a second configuration;

FIG. 31 illustrates a further exemplary test gas charging system in a first configuration;

FIG. 32 illustrates yet another exemplary test gas charging system in a first configuration;

FIG. 33 illustrates still another exemplary test gas charging system in a first configuration; and

FIG. 34 illustrates an exemplary particle generator.

Corresponding reference characters indicate corresponding parts throughout the several views. Although the drawings represent embodiments of the present disclosure, the drawings are not necessarily to scale, and certain features may be exaggerated in order to better illustrate and explain the present disclosure. The exemplification set out herein illustrates an embodiment of the disclosure, in one form, and such exemplifications are not to be construed as limiting the scope of the disclosure in any manner.

### DETAILED DESCRIPTION

Reference will now be made in detail to certain embodiments of the disclosed subject matter, examples of which are illustrated in part in the accompanying drawings. The embodiments disclosed herein are not intended to be exhaustive or limit the present disclosure to the precise form disclosed in the following detailed description. Rather, the embodiments are chosen and described so that others skilled in the art may utilize their teachings. Therefore, no limitation of the scope of the present disclosure is thereby intended.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In some instances throughout this disclosure and in the claims, numeric terminology, such as first, second, third, and fourth, is used in reference to various components or features. Such use is not intended to denote an ordering of the components or features. Rather, numeric terminology is used to assist the reader in identifying the component or features being referenced and should not be narrowly interpreted as providing a specific order of components or features.

With respect to the terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts, such as the aerosol arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

As used herein, the term “space” refers to the area of space being cleaned by the one or more mitigation systemss. A “space” can be an indoor environment, such as a room. The air of a “space” or “air space” refers to an area of air being cleaned by the one or more mitigation systemss. As used herein, the terms, “indoor environment”, “indoor space” or “room” refers to a bound space benefiting from one or more particle mitigation systems. Typically, the air of such spaces readily mixes such that subjecting one portion of air in the space to cleaning by the mitigation systems will impart its effect on the entirety of the space. An indoor environment is separated from an exterior environment such as the outdoors except via sources of ventilation, which includes apparent sources of ventilation (e.g., windows and air handler ventilation) and non-apparent sources of ventilation (e.g., leakage). In the case of a room, the space or air space will typically refer to the entire volume of the air in the room as conventionally described by the boundary of the room (e.g., as defined by its walls, doors, and windows). Suitable spaces include, but are not limited to, those in restaurants, schools, workplaces, offices, hospitals, cafeterias, event space, bars, theaters, gyms, retail space, public transportation vehicles, public transportation stations, airports, and airplanes. In various embodiments, the indoor environment can be an occupiable space, an occupied space, a space having occupants come and go, a space having occupant activity, a space having occupant movement, a space having occupants that increase or decrease ventilation, a space having activities that increase or decrease ventilation, a space having fluctuating ventilation, a space having uncontrolled ventilation, or a space having an air volume determined by a method described herein.

As used herein, “ACH” is a measure of the rate at which the air volume of a space is mechanically exchanged with ‘outside’ clean air. ACH can be determined from cubic feet of ventilation per minute (CFM)×60/volume of the space. ACH can be better understood by referring to ASHRAE Standard 62, ASHRAE Standard 62.1 and/or ANSI Standard 136, each of which are incorporated by reference herewith. In various contexts, those skilled in the art will understand numerous other equivalents, some of which may be expressed in differing units, scales, or terms while nonetheless being substantially interchangeable expressions. In various contexts, ACH refers to air exchange between the indoor environment and exterior spaces (e.g., the outdoors), which includes apparent sources of ventilation (e.g., windows and air handler ventilation) and non-apparent sources of ventilation (e.g., leakage), but is intended herein to refer to ventilation-based effects not the effects of particle mitigation systems like filters. When using a gas to assess ventilation, in various aspects it can be advantageous to use at least one gas that is inert to particle mitigation efforts such as mechanical filters, scrubbers, and UV light system. When monitoring and analyzing different gases, different ACH values may result, which can reflect differences in non-ventilation gas removal processes if one gas is inert to particle mitigation and the other gas is not. For example, measuring ACH for an indoor space with a particle mitigation system by using both iPrOH and CO2, will provide ACH values for each (e.g., ACH_{iPrOH} and ACH_{CO2}); a high ACH for isopropanol can reflect a contribution from particle mitigation systems that is removing iPrOH but not removing CO2, e.g., a scrubber. In such cases, the ACH can be determined for the indoor space over all (e.g., ACHᵣₒₒₘ), which can be used as a consistent ventilation contribution relevant to all gases and particles in the space, except that some particles will be additionally mitigated by particle mitigation systems and some gases might be mitigated by a scrubbers, each of which effects are best described by using MEACH rather than ACH). Herein, ACH is particularly useful when describing ventilation, since ventilation can be determined by using a gas but then applied as an adjustment for particles in order to determine mitigation equivalent air change rate of an indoor environment's particle mitigation systems, or one or more portion thereof.

As used herein, “mitigation equivalent air change rate” or “MEACH” is a measure of the rate at which particles are removed air in a mitigated space, or the rate at which particles are removed by the particle mitigation systems or portion thereof, in a space in a manner equivalent to air exchange with clean outside air. As such, any given determination of MEACH is relative to (1) the referenced system, such as the room overall, the particle mitigation systems for the room, or one or more portion of the particle mitigation systems, and (2) the measured particles. That is, in one context MEACH can be used to describe total particle removal rate, such as determined from the decay rate of particles charged in the room. Mitigation Because gas is normally not removed by particle mitigation systems, typically gas removal would be described using ACH (a ventilation process) and would not be described using MEACH (which references contribution of particle mitigation systems). Examples of referenced particle mitigation systems, or portion thereof, that may be useful to describe using a MEACH include all particle removal for the indoor environment overall other than ventilation, all particle mitigation systems in the environment, one or more particle type of particle mitigation technique for the room overall, one or more particle physically coupled, branded, or grouped particle mitigation system for the room overall (e.g., HVAC, portable purifier system, window unit, etc.), mechanical filtration systems, electrostatic systems, germicidal light systems, air handling systems, air handling systems including any ventilation-based activity involved therein, and air handling systems not including any ventilation-based activity involved therein. Mechanical filtration systems remove particles at various rates depending on size from the air within the indoor environment. Mechanical filtration systems can be rated according to MERV or HEPA systems. Mechanical filtration systems may be stand-alone units positioned within the indoor environment or integrated into the air handling system as part of the recirculation air flow path of the air handling system. Another exemplary particle mitigation system is a light source to which the target particle is sensitive to and resulting in a destruction of the target particle, such as germicidal ultraviolet radiation. A further exemplary particle mitigation system is an electrostatic system which ionizes the target particle, enhancing particle collection through electrostatic forces. Examples of references particles can include non-gas particles, particles of the type mitigated by the particle mitigation system, particles of the type monitored by the one or more particle monitor, PM0.1, PM1.0, PM2.5, PM5, PM10, PN0.1, PN1.0, PN2.5, PN5, PN10, aqueous mineral salt solutions, sodium chloride (NaCl), potassium chloride (KCl), Oleic Acid, Polystyrene Latex Spheres, Mineral Oil, Propylene Glycol, Saccharin (or other artificial sweeteners), Sucrose, Fructose, light-sensitive particles, non-infectious viral particle models, oxidized iodide compounds, CBr₄, non-toxic and non-peptide fluorophores, DNA-tagged molecules, fluorescent tagged proteins, or any combination thereof. Although any given MEACH data is made with reference to a given set of measured particles, the result is useful for determining air quality performance for the room or mitigation systems for other particles as well. In various aspects, each of the presently described reference particles can be suitable used as model for determining air quality and performance of a room or mitigation system for other particles, particularly the undesirable, unhealthy, or harmful particles for which mitigation is primarily sought. For instance, infectious particles are difficult to measure and monitor, however, other particle such as those described herein can be readily monitored to determine particle removal rate of those particles, CADR or other values indicative of airflow, particle flow, air exchange, or air quality. When appropriate, the resulting values can be adjusted with a conversion factor to better reflect performance with respect to a given set of particles of interest or concern. A typical conversion factor is to utilize particle removal rate to determine CADR and then subsequently determine a value describing infection risk in the space.

As described herein, the MEACH of the given particle mitigation systems, or portion thereof, is typically determined from the total particle removal rate in the room and subtracting the contribution of other particle removal mechanisms such as ventilation. For instance, the MEACH for a plurality of particle mitigation systems with respect to PM2.5 reflects the contribution of the particle mitigation systems to the removal of PM2.5 from the air, which can be determined by calculating PM2.5 removal rate for the indoor environment overall and subtracting the effects of other PM2.5 removal mechanisms such as ventilation. As another example, the MEACH for a plurality of particle mitigation systems with respect to a light-sensitive particle may reflect the contribution of a light-system portion of a particle mitigation system with respect to removal of the light-sensitive particles from the air, which can be determined by calculating overall removal rate for the light sensitive particles in indoor environment and then subtracting the effects of other light-sensitive particle removal mechanisms such as ventilation, or even filtration if previously determined. Indeed, individual portions of particle mitigation systems can be resolved by using time-coordinated measurements of both particle mitigation and ventilation (via gas); and multiple particle mitigation strategies can be determined by first ascertaining the effect of one technique (e.g., mechanical filtration), then other techniques (germicidal light). To disambiguate between mechanisms like particle filtration components, electrostatic systems, or light systems, an A-B approach would be used where filtration is determined while germicidal lights are off, and then lights are turned on and effect of germicidal lights can be determined-all from the total particle change in the room together with operatively coupled ventilation determination via gas measurement.

The presently described systems and methods provide a means for obtaining at least: (1) overall particle removal (for clarity, MEACHᵣₒₒₘ), (2) ventilation (for clarity, ACHᵣₒₒₘ), and thus also (3) the amount of particle removal contributed by the non-ventilation mechanism (for clarity, MEACH_{system}), which can be useful for assessing how much the particle mitigation systems help relative to ventilation, or when one or more adjustments are appropriate. Other prior systems that fail to utilize coordinated measurement of ventilation must use restrictive testing conditions or obtain results that fail to account for ventilation or fluctuations thereof.

The methods and systems described herein relate to particle mitigation systems, which generally serve to remove a broad range of particles from the air of an indoor space. In various aspects, the methods and systems involve monitoring particular particles or collection of particles in order to analyze and assess various air characteristics of the indoor space and the particle mitigation systems applied therein. In this document, the particles that are monitored may occasionally be referred to as test particles, regardless of whether they are provided as part of a formalized test routine or whether or not they are provided by the user. Moreover, the particles that are monitored are not necessarily the particles for which mitigation systems are primarily employed. For example, particles utilized as test particles are typically non-toxic and utilized at safe levels, yet the resulting assessment of air quality and particle mitigation system performance is relevant for assessing removal of other more harmful particles-such as infectious particles and toxic pollutants.

The term “particle mitigation systems” refers to systems configured for removing undesirable, unhealthy, or harmful particles from the air of an environment. Particle mitigation systems often utilize air flow to transfer undesired particles away from a given space, or transfer the particles to a device that captures (e.g., a filter) or destroys them (e.g., UV light). Moreover, while particle mitigation systems are primarily configured for removing undesired particles from air, they may also involve ventilation techniques that remove and exchange the air overall. A given set of particle mitigation systems can have multiple portions that each serve to impart some effect on removing undesired particles from air. A “portion” of one or more particle mitigation system refers to an operable component of such system that functions for removing undesirable, unhealthy, or harmful particles from the air of an environment. For example, some particle mitigation systems can include multiple filtration systems, an air handling system that exchanges indoor air with fresh outdoor air, and a UV light system for destroy infectious particles. An air handling system typically includes one or more fan. An air handling system can be an example of a particle mitigation system when it includes or facilitates particle removal. An air handling system can also provide ventilation, which would contribute to particle removal overall. The particle removing effects of an air handling system can be described in terms of non-ventilation particle removal and ventilation-based particle removal. The “portions” of a plurality of particle mitigation systems can refer to components thereof in terms of any of function, mitigation technique, physical position, or other form of classification. The “portions” of a plurality of particle mitigation systems can thus refer to any of the following: a portion corresponding to each individual particle mitigation system; the filtration portion of the plurality of particle mitigation systems over all or of an individual particle mitigation system contained therein; the air handling system portion of the plurality of particle mitigation systems over all or of an individual particle mitigation system contained therein; the ventilation or air exchange portion of the plurality of particle mitigation systems over all or of an individual particle mitigation system contained therein; the light system portion of the plurality of particle mitigation systems over all or of an individual particle mitigation system contained therein; individual filters or groups of filters; individual ventilation sources or groups of ventilation sources; individual light sources or groups of light sources, or any combination thereof. The presently described systems and methods are particularly useful for resolving the efficacy and performance of discrete portions of particle mitigation systems. In aspects, various systems and methods described herein can advantageously determine the efficacy or performance of a given particle mitigation system, or portion thereof, in environments having ventilation, occupancy, or varying conflating conditions.

The particles removed by particle mitigation systems can include airborne infectious agents, pollutants, and other contaminants. Undesirable airborne particles can be characterized in terms of particle size or particle size ranges, which informs the nature of their health effects and how such particles can be removed. It should also be understood that particles can refer to a given collection of different molecules (e.g., PM2.5) and that various conceptual subsets of aerosol contaminant exist (e.g., PM5 or less). For example, undesirable particles can include particulate matter characterized as PM0.1, PM1.0, PM2.5, PM5, PM10, PN0.1, PN1.0, PN2.5, PN5, PN10, or any combination thereof. PM2.5 and PM10 are used describe the total particle mass for sizes less than 2.5 μm and 10 μm, respectively. Undesirable particles can also be characterized in terms of density, optical size or other optical properties, particle size, particle size distribution, surface area, charge to mass ratio, a charge segregated criteria, or a size segregated criteria. Further examples of particles removed by particle mitigation systems includes smoke, dust, or pollen. Further examples of particles can include products of combustion or combustion-based activities, including diesel and gasoline emission sources, cigarette smoke, wildfire smoke, smog, cooking particles, or components thereof. The particles can include pathogenic particles and non-pathogenic particles. In the context of infectious diseases, people talking or breathing can represent sources of pathogenic particles that are undesirable in air.

The systems and methods described herein are useful for determining the efficacy and performance of one or more particle mitigation systems for a given set of particles. The relevant particles include particles that are removed via a given particle mitigation system or portion thereof. As one non-limiting example, the efficacy of many commercially available particle filters (e.g., HEPA filters) is based on removing particles that are 0.3 microns or larger in size; thus, such particles are a particularly useful reference for assessing particle mitigation systems.

The term “particles” as used herein can have different meanings depending on context. Typically, in an air mitigation context, the term particles refers to undesirable particles such as particulate matter of a sufficient character that it can be destroyed, eliminated, or otherwise removed, via a particle mitigation system. In another context, all airborne constituents are technically particles in a physical sense, whether they are a gas molecule particle that would not be readily filtered or a readily filter collection of particulate matter (e.g., PM2.5). Thus, to clarify this point, reference is sometimes made herein to two sets of such particles: a set of particles that are more readily removed via filtration and a set of particles that are less readily removed via a given mitigation system. For example, a mechanical filter will typically be effective to remove PM10 particle matter from air, but it will not typically be effective to remove a gas such as CO2 from the same air. In contrast, a ventilation system, or the ventilation component of a particle mitigation system, will generally remove all airborne contaminants. A filter may remove particle matter but not CO2, while ventilation removes both particle matter and CO2. Lastly, when a portion of a particle mitigation device selectively removes a gas molecules (e.g., VOCs removed by a scrubber), such molecules may be described as particles to the extent that they are removed via a non-ventilation portion of the particle mitigation portion of the particle mitigation system; for example, a scrubber will have a mitigation equivalent air change rate (MEACH) for isopropanol and other VOCs when those gas molecules be mitigatable species mitigated by the particle mitigation system.

The systems and methods described herein utilize gas monitoring in order to account for air flow, ventilation, room effects, and varying conflating conditions that can significantly affect accuracy of assessing mitigation efforts for removing given non-gas particles. As used herein “gas” refers to an air component in the gaseous or vaporous fundamental state of matter. Typically, gases will be small molecules that are not readily removed via a particle mitigation system. Gases include carbon dioxide (CO₂); organic molecules including volatile organic compounds (VOCs), such as methane, butane propoane, 2-propanol, ethanol, formaldehyde, ammonia; water vapor (H₂O); radon; oxygen (¹⁸O₂); ozone (O₃); heavy water (D₂O); Nobile gases such helium (He), Xenon, and Argon; sulphur hexafluoride (SF₆); Dimethydinitrobutane; Tetrafluoroethane; Tetrafluoropropene; and other suitable test gases.

As used herein, an “electronic controller” or “control system” is a machine, or group of machines, which functions to operatively couple one, two, or more components of the system together. The control system may send, receive, or act upon instructions, data, or signals transmitted between another component system (e.g., particle monitors, gas monitors, sensors, and the like). In various examples, the control system can direct and coordinate operation of a particle monitor and a gas monitor so that the monitors work together in concert, such as by perform a one or more monitoring simultaneously, sequentially, contemporaneously, or in at least partially overlapping timeframes. In further examples the control system can serve to operatively couple a particle monitor and a gas monitor by receiving and storing data collected from each monitor. In yet further examples the control system can serve to operatively couple a particle monitor and a gas monitor by processing data collected from each monitor. Typically, the control system mediates gas and particle monitoring and records data received from the gas and particle monitors. The control system can serve as an interface for time-coordinated generation, monitoring, data collection, or recording efforts such that the resulting data is coordinated time series data.

In various aspects, the control system can execute instructions embodying any one or more of the methodologies or functions described herein. The control system can include a memory that can store instructions or data structures (e.g., software) embodying any one or more of the methodologies or functions described herein. The control system can include one or more hardware component or software component, each of which may be a dedicated or multipurpose device specifically configured for one or more of the methodologies or functions described herein. The control system can be a single unit or it can be multiple units. The activities performed by the control system can thus be distributed across multiple units, including in a configuration where each unit serves a different purposes and provides a different contribution to any given set of activities.

The terms “couples”, “coupled”, “coupler” and variations thereof are used to include both arrangements wherein the two or more components are in direct physical contact and arrangements wherein the two or more components are not in direct contact with each other (e.g., the components are “coupled” via at least a third component), but yet still cooperate or interact with each other. Thus, components may be coupled via a physical, cooperative, interactive, operational, functional, electrical, or communicative (including wireless) relationship. Examples include components in a shared housing, integrated with a shared computer system or control system, and components that are coordinated to operate together. The phrase “operatively coupled” refers to components that are configured to work together. Examples of operatively coupled components include components in a system configured such that the components operate simultaneously, sequentially, contemporaneously, or in at least partially overlapping timeframes. For example, a gas monitor and a particle monitor can be configured record data simultaneously, sequentially, contemporaneously, or in at least partially overlapping timeframes. As another example, a gas generator and a particle generator can be configured to initiate or terminate generation simultaneously, sequentially, contemporaneously, or in at least partially overlapping timeframes. As yet another example, a gas monitor and a particle generator can be configured so the particle generator initiates or terminates generation simultaneously, sequentially, contemporaneously, or in at least partially overlapping timeframes with detection of a given gas characteristic measurement or change. As yet another example, a gas monitor and a occupancy sensor can be configured so the gas monitor monitors, records, or transmits gas characteristic data simultaneously, sequentially, contemporaneously, or in at least partially overlapping timeframes with detection of a given occupancy measurement or change. As yet another example, a particle monitor and a occupancy sensor can be configured so the particle monitor monitors, records, or transmits gas characteristic data simultaneously, sequentially, contemporaneously, or in at least partially overlapping timeframes with detection of a given occupancy measurement or change. Examples of operating with overlapping timeframes includes time frames for generation, monitoring, or recording, or overlapping timeframes of time stamped data selected for analyzing, recording, or processing, which having times that overlap by up to, at least, or about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 120, 180, 240, 300, 360, 420, 480, 540, 600, 900, or 1200 seconds; up to, at least, or about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 120, 180, 240, 300, 360, 420, 480, 540, 600, 900, 1200, or 1440 minutes; or up to, at least, or about 1, 2, 3, 4, 5, 6, 7, 8, 9, 10, 11, 12, 13, 14, 15, 16, 17, 18, 19, 20, 21, 22, 23, 24, 36, 48, 60, 72, 84, 96, 108, or 120 hours.

The term “air characteristic” of the indoor environment is utilized herein as a general term representing a characterization of the indoor environment, its particle mitigation efforts, or its air quality, determined based on gas monitoring, particle monitoring, or both. Exemplary terms are particle concentration, particle number, particle removal rate, gas concentration, gas number, gas removal rate, CADR (for room overall), ACH (for all ventilation in room), MEACH (for all particle mitigation systems of room, or portion thereof), infection risk (for room overall), and portion of infection risk addressed by particle mitigation systems. In various examples, such one or more air characteristics are adjusted on a per occupant basis, such as particle concentration, particle number, particle removal rate, gas concentration, gas number, gas removal rate, CADR (for room overall), ACH (for all ventilation in room), MEACH (for all particle mitigation systems of room, or portion thereof), infection risk (for room overall), and portion of infection risk addressed by particle mitigation systems, each per occupant.

Air characteristics can include air quality values, airflow-based values, and equivalents thereof. For example, current air-change rate, current particle clearance rate, time to achieve a given air quality, time to achieve a given threshold of particle clearance (e.g., 99% clearance), time to achieve a given amount of clean air replacement, and time to achieve a given volume of clean air replacement, for which it should be recognized that MEACH values serve as equivalent contributors to air exchange in addition to the effects of ventilation. Further examples include safety-based metrics derived from threshold exposure limits, such as expected exposure, total exposure, peak exposure, exposure over a particle amount of time such as a work day, chronic exposure, expected health risk due to exposure, expected life years lost due to exposure. As further examples, air characteristics can include infection risk, expected infection rate, expected infection number, expected infection reduction rate, expected exposure number, exposure risk, expected exposure rate, expected exposure reduction rate, and composite scores derived from any of the aforementioned values.

Further examples include infection-based metrics derived from air quality values, airflow-based values, and their equivalents. For example, air characteristics can include infection risk, expected infection rate, expected infection number, expected infection reduction rate, expected exposure number, exposure risk, expected exposure rate, expected exposure reduction rate, and composite scores derived from any of the aforementioned values.

Further examples include composite scores, pass assessments, or fail assessments, based on comparison test against a target criteria or threshold value related to air quality or particle mitigation. In some aspects, the criteria or threshold value can be adjusted based on occupant number or activity, or current outdoor air pollution. In some embodiments, a pass assessment, fail assessment, or result of a comparison test can take the form of an instruction for altering a setting of a particle mitigation system or ventilation system.

The term “particle characteristic” is utilized herein as a general term representing the amount, number, effect, character, decay rate, of the referenced particles. Examples include particle concentration and particle number in the air of the indoor environment. Further examples include particle removal rate in the indoor environment, particle removal rate based on ventilation, and particle removal rate based on one or more particle mitigation systems or portion thereof. Particle removal rates can be determined by collecting and recording particle data (e.g., concentrations or number) and then determining an exponential fit of a decay curve to determine particle removal rate. Other techniques are also available, such as are described in ASHRAE 241.

The term “gas characteristic” is utilized herein as a general term representing the amount, number, effect, character, decay rate, of the referenced gas. Examples include gas concentration and gas number in the air of the indoor environment. Further examples include gas removal rate in the indoor environment, gas removal rate based on ventilation, and gas removal rate based on one or more gas mitigation systems or portion thereof. Gas removal rates can be determined by collecting and recording gas data (e.g., concentrations or number) and then determining an exponential fit of a decay curve to determine particle removal rate. Other techniques are also available, such as are described in ASHRAE 241 or ASTM E741.

As used herein, the term “pathogen”, “pathogenic particle” and “infectious particle” includes viruses, bacteria, infectious microbes, fungi, protozoa, parasites, and other agents, or components thereof, that are capable of causing disease or infection in humans or animals. The term “destroy” in the context of infectious particles refers to inactivating the particle so that it is no longer infectious. The term “pathogen” includes pathogens in droplets, pathogens in aerosols, pathogens on surfaces, and free pathogens in air. Examples of pathogen include SARS-COV-2, influenza, varicella, and RSV. The methods and systems described herein obtain one or more air characteristic of the indoor environment (e.g., CADR, CADR per occupant, MEACH+ACH, etc) that can be useful for determining particle removal in a space, including mitigation of infectious particle. Various known methods can be utilized to determine results related to infection risk, most of which require such air characteristics of a given space in order to determine removal rate of infectious particles by particle mitigation systems in place. Moreover, some methods can benefit from utilizing occupancy and occupant activity in addition to utilizing air-flow, air quality, and particle mitigation type values such as CADR or CADR per occupant. See, Sze To G N, Chao C Y. Review and comparison between the Wells-Riley and dose-response approaches to risk assessment of infectious respiratory diseases. Indoor Air. 2010 February; 20 (1): 2-16 and Z. Peng, A. L. Pineda Rojas, E. Kropff, W. Bahnfleth, G. Buonanno, S. J. Dancer, J. Kurnitski, Y. Li, M. G. L. C. Loomans, L. C. Marr, L. Morawska, W. Nazaroff, C. Noakes, X. Querol, C. Sekhar, R. Tellier, T. Greenhalgh, L. Bourouiba, A. Boerstra, J. W. Tang, S. L. Miller, and J. L. Jimenez, Practical Indicators for Risk of Airborne Transmission in Shared Indoor Environments and Their Application to COVID-19 Outbreaks, Environmental Science & Technology 2022 56 (2), 1125-1137, copies of which are incorporated by reference herewith in their entireties. See, also, A variety of other models can be used to determine the probability of infection. For example, by assumptions of steady state (See, Peng et al. 2022), or by using time and initial concentration in the model to better reflect the build up of infectious aerosols in a space, as well as the bleed down when an infectious person leaves. See, Noakes C J, Beggs C B, Sleigh P A, Kerr K G. Modelling the transmission of airborne infections in enclosed spaces. Epidemiol Infect. 2006 October; 134 (5): 1082-91, which is incorporated by reference herewith in its entirety.

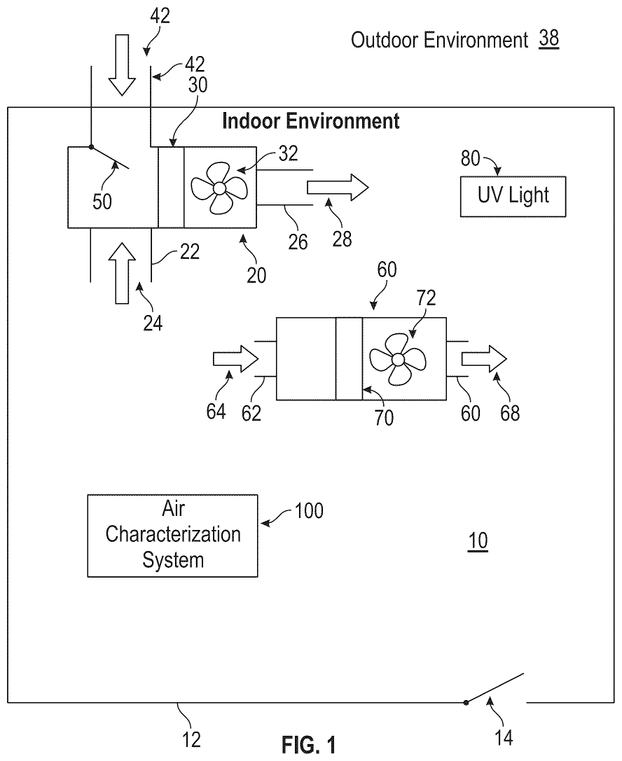

Referring to FIG. 1, an exemplary indoor environment 10 is illustrated. Indoor environment 10 is generally a bounded space that includes an air volume. As illustrated, indoor environment 10 includes a set of walls 12 and a door 14 provided in the set of walls to provide ingress and egress from indoor environment 10. Although a single door 14 is shown, indoor environment 10 may include multiple doors 14. Further, although not illustrated, indoor environment 10 may include one or more windows which, in embodiments, may be opened to provide air exchange with an outdoor environment. Indoor environment 10 includes a plurality of particle mitigation systems to lower the number of particles in indoor environment 10.

Further referring to FIG. 1, the drawing illustrates exemplary particle mitigation systems. A first exemplary particle mitigation system is an air handling system 20. Air handling system 20 includes a first fluid conduit 22 having at least one opening 24 in fluid communication with indoor environment 10. Although a single fluid conduit 22 and opening 24 are shown, in embodiments, a given fluid conduit 22 may have multiple openings 24 and system may include multiple fluid conduits 22 each of which have one or more openings 24. Air is drawn into an interior air handling system 20 by a fan 32 or other air moving device and out through a second fluid conduit 26 back into indoor environment 10 through opening 28. Although a single fluid conduit 26 and opening 28 are shown, in embodiments, a given fluid conduit 26 may have multiple openings 28 and system may include multiple fluid conduits 26 each of which have one or more openings 28. In embodiments, as air passing through the interior of air handling system 20, it passes through one or more mechanical filters 30 which filter particles out of the air stream above a first threshold. Exemplary filters have a minimum efficiency reporting value (MERV) in the range of 1-16. A typical residential filter may have a MERV value of 13 which only filters about 50% of particles in the range of 0.3-1.0 microns.

An advantage, among others of recirculating air from indoor environment 10 back to indoor environment 10 is that the air does not require additional heating or cooling as it is already at the set temperature desired for indoor environment 10. In embodiments, air handling system 20 further includes a third fluid conduit 40 having at least one opening 42 which is in fluid communication with outdoor environment 38. Although a single fluid conduit 40 and opening 42 are shown, in embodiments, a given fluid conduit 40 may have multiple openings 42 and system may include multiple fluid conduits 40 each of which have one or more openings 42. Air handling system 20 includes a damper 50 which is movable to control a ratio of air from third fluid conduit 40 that is allowed to mix with air from first fluid conduit 22.

A second exemplary particle mitigation system is an air purifier 60. Air purifier 60 includes a first fluid conduit 62 having at least one opening 64 in fluid communication with indoor environment 10. Although a single fluid conduit 62 and opening 64 are shown, in embodiments, a given fluid conduit 62 may have multiple openings 64 and system may include multiple fluid conduits 62 each of which have one or more openings 64. Air is drawn into an interior air purifier 60 by a fan 72 or other air moving device and out through a second fluid conduit 66 back into indoor environment 10 through opening 68. Although a single fluid conduit 66 and opening 68 are shown, in embodiments, a given fluid conduit 66 may have multiple openings 68 and system may include multiple fluid conduits 66 each of which have one or more openings 68. In embodiments, as air passing through the interior of air purifier 60, it passes through one or more mechanical filters 70 which filter particles out of the air stream above a first threshold. Exemplary filters include high efficiency particulate air filters (HEPA). An exemplary HEPA filter may have a filter profile to capture 99.7% of all particles having a size of 0.3 microns and higher. Other exemplary filters may have a filter profile a filter profile that is most effective at filtering both large particles above a first threshold and small particles below a second threshold and least effective at particles between the first threshold and the second threshold.

A third exemplary particle mitigation system is a light 80. Light 80 may include a single light source or multiple lights sources. Light 80 emits radiation that is targeted to destroy or otherwise inactivate specific particles. In embodiments, light 80 emits radiation having a wavelength of about 222 nanometers (nm). In embodiments, light 80 is a germicidal UV light.

Indoor environment 10 additionally includes an air characterization system 100. In embodiments, air characterization system 100 is a permanent part of indoor environment 10. In embodiments, air characterization system 100 is mobile and may be temporarily placed in indoor environment 10. Although indoor environment 10 is shown as unoccupied in FIG. 1, air characterization system 100 is capable of operating effectively when people are present in indoor environment 10.

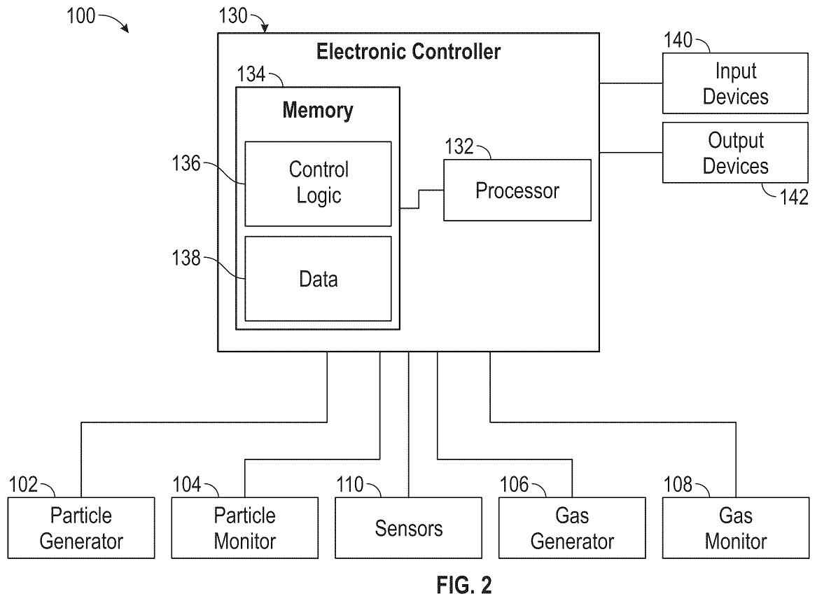

Referring to FIG. 2, an exemplary air characterization system 100 is shown. Air characterization system 100 includes a particle generator 102, a particle monitor 104, a gas generator 106, a gas monitor 108, and sensors 110. Although a single particle generator 102, particle monitor 104, gas generator 106, and gas monitor 108 are shown, multiple instances of each may be included in air characterization system 100.

Additionally, air characterization system 100 includes an electronic controller 130. Electronic controller 130 includes at least one processor 132 and associated memory 134. Memory 134 includes control logic 136 and data 138. Exemplary control logic includes logic to control the operation of one or more of particle generator 102, particle monitor 104, gas generator 106, and gas monitor 108, to provide analysis results to an operator, to provide recommendations to an operator, and/or control the operation of one or more of air handling system 20, air purifier 60, and light 80. The term “logic” as used herein includes software and/or firmware executing on one or more programmable processors, application-specific integrated circuits, field-programmable gate arrays, digital signal processors, hardwired logic, or combinations thereof. Therefore, in accordance with the embodiments, various logic may be implemented in any appropriate fashion and would remain in accordance with the embodiments herein disclosed. A non-transitory machine-readable medium comprising logic can additionally be considered to be embodied within any tangible form of a computer-readable carrier, such as solid-state memory, magnetic disk, and optical disk containing an appropriate set of computer instructions and data structures that would cause a processor to carry out the techniques described herein. This disclosure contemplates other embodiments in which electronic controller 130 is not microprocessor-based, but rather is based on one or more sets of hardwired instructions. Further, electronic controller 130 may be contained within a single device or be a plurality of devices networked together or otherwise electrically connected to provide the functionality described herein. The processing sequences described herein and the operations of electronic controller 130 may be performed by multiple processors and involve the access to and storage on multiple memories.

Electronic controller 130 may further receive input through one or more input devices 140. Exemplary input devices include buttons, switches, levers, dials, touch displays, soft keys, and a communication module, such as a network device (e.g. ethernet connection, wireless network transceiver, a cellular transceiver, a satellite transceiver, and other suitable devices which allow electronic controller 130 to communicate with other computing devices). Electronic controller 130 may further provide output through one or more output devices 142. Exemplary output devices include visual indicators, audio indicators, and a communication module. Exemplary visual indicators include displays, lights, and other visual systems. Exemplary audio indicators include speakers and other suitable audio systems. Exemplary communication module, such as a network device (e.g. ethernet connection, wireless network transceiver, a cellular transceiver, a satellite transceiver, and other suitable devices which allow electronic controller 130 to communicate with other computing devices).

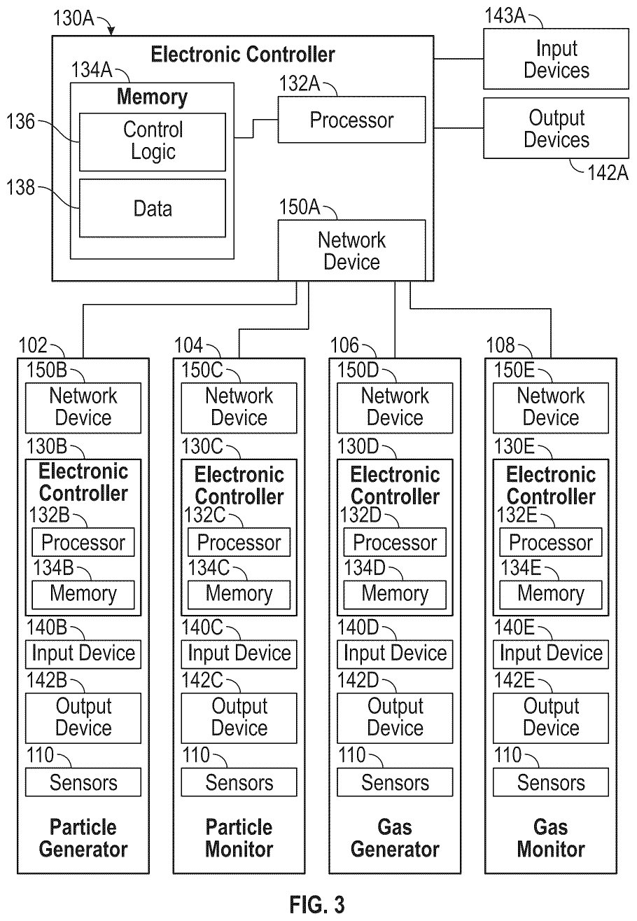

Referring to FIG. 3, an embodiment of air characterization system 100, wherein each of particle generator 102, particle monitor 104, gas generator 106, and gas monitor 108 include a portion of electronic controller 130, designated as electronic controller 130B-E, is shown. Further, control logic 136 and data 138 may be distributed among electronic controller 130A which serves as a main electronic controller 130 of air characterization system 100 and each of electronic controllers 130B-E. Electronic controller 130A and particle generator 102, particle monitor 104, gas generator 106, and gas monitor 108 are coupled together through network devices 150A-E, such as through a wireless network, a wired network, or a combination thereof.

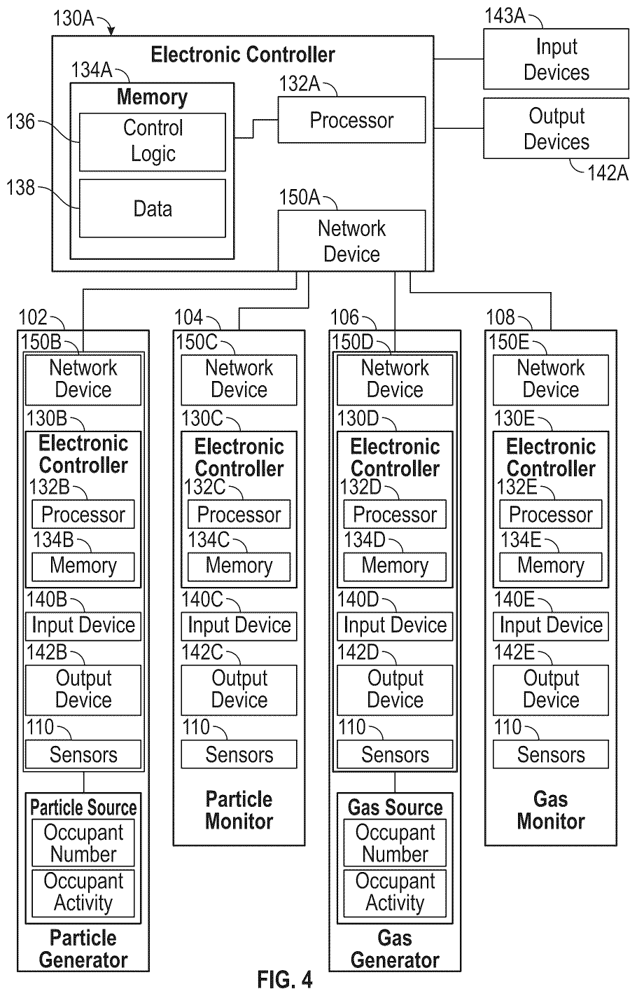

Referring to FIG. 4, an embodiment of air characterization system 100, wherein each of particle generator 102, particle monitor 104, gas generator 106, and gas monitor 108 include a portion of electronic controller 130, designated as electronic controller 130B-E, is shown. Further, control logic 136 and data 138 may be distributed among electronic controller 130A which serves as a main electronic controller 130 of air characterization system 100 and each of electronic controllers 130B-E. Electronic controller 130A and particle generator 102, particle monitor 104, gas generator 106, and gas monitor 108 are coupled together through network devices 150A-E, such as through a wireless network, a wired network, or a combination thereof. Particle generator 102 sensors 110 are utilized to detect particle generating events in the indoor environment, particularly particle generation from sources such as human occupant activities such as cooking, cleaning, movement, or work. The particle generator 102 sensors 110 can include occupancy sensors for detecting occupant activity and number. This type of configuration permits use of occasional intermittent or regular occupant activities as particle sources. Gas generator 108 sensors 110 are utilized to detect gas generating events in the indoor environment, particularly gas generation from occupants breathing or engaging in activities that result in greater respiration such as exercise or vigorous activity. The gas generator 108 sensors 110 can include occupancy sensors for detecting occupant activity and number. This type of configuration permits use of occupants, which may come and go or vary their activities, as gas sources.

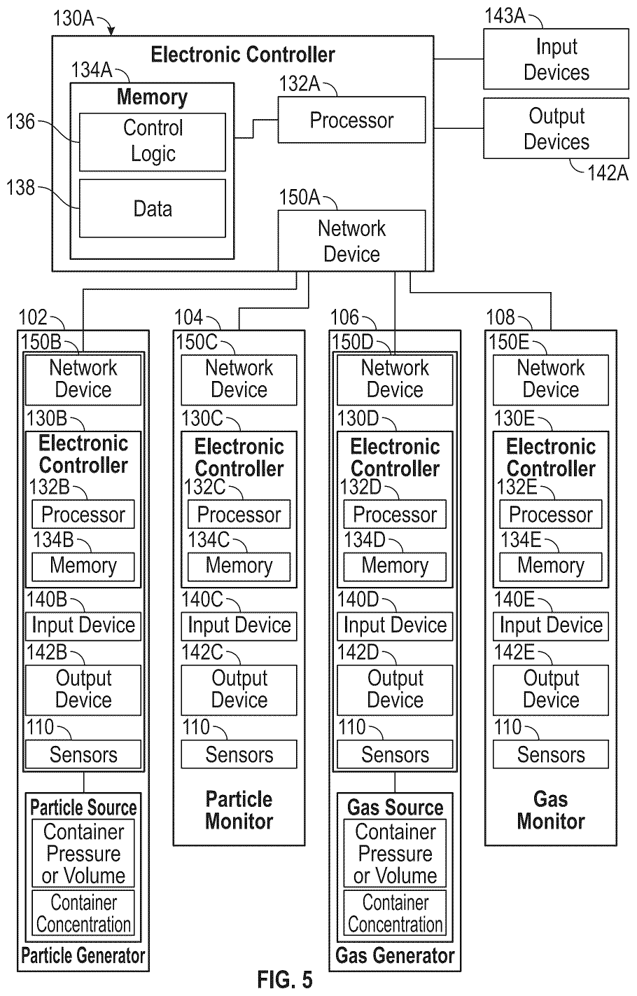

Referring to FIG. 5, an embodiment of air characterization system 100, wherein each of particle generator 102, particle monitor 104, gas generator 106, and gas monitor 108 include a portion of electronic controller 130, designated as electronic controller 130B-E, is shown. Further, control logic 136 and data 138 may be distributed among electronic controller 130A which serves as a main electronic controller 130 of air characterization system 100 and each of electronic controllers 130B-E. Electronic controller 130A and particle generator 102, particle monitor 104, gas generator 106, and gas monitor 108 are coupled together through network devices 150A-E, such as through a wireless network, a wired network, or a combination thereof. Particle generator 102 sensors 110 are utilized to reliably determine the amount of particles provided to the space from a controlled source, such as a containers. The particle generator 102 sensors 110 can include pressure, volume, or concentration sensors for detecting the amount of particles released from a container or remaining in a container. This type of configuration permits use of a known bolus of particles provided to the indoor environment. Gas generator 108 sensors 110 can include pressure, volume, mass, and/or concentration sensors for detecting the amount of gas released from a container or remaining in a container. This type of configuration permits use of a known bolus of gas provided to the indoor environment. In various aspects, the particle generator 102 and the gas generator 106 can utilize a shared source, such as a container that contains both gas (or gas precursors) and particles (or particle precursors) for delivery simultaneously, sequentially, or otherwise time-coordinated to the indoor environment.

A known bolus can be used to determine the volume of air in the indoor environment, which is the effective room size. For example, the air volume of the indoor environment can be readily back calculated from the gas concentration detected in the indoor environment following supply of a known amount of gas when the amount of ventilation is insignificant or by accounting for the amount of any ventilation. As another example, the air volume of the indoor environment can be calculated by using the time to return to baseline after providing a known amount of gas together with using the ventilation rate determined by fitting an exponential to the decay curve of the resulting bolus. This approach can avoid the need for physical measurement of the indoor environment and moreover has the advantage of accounting for objects that reduce the total air volume in the indoor environment. Additionally, the air volume can be determined by measuring the starting, or baseline concentration, and then recording the resulting increase in concentration in the space, given the total mass or volume released in the space, accounting for removal by ventilation. The resulting air volume value can be utilized in each of the various systems and methods described herein whenever volume of the indoor environment is utilized, for example when determining an air characteristic, a particle characteristic, a gas characteristic, particle concentration, particle number, gas concentration, gas number, ACH, MEACH, CADR, or CADR per occupant values. In some embodiments, a gas generator or a particle generator can be configured to provide a known bolus of the gas or particles at the same time, sequentially, before, after, or otherwise time-coordinated with gas monitoring or particle monitoring. In some embodiments, a gas generator or a particle generator can be configured to provide a known bolus of the gas at the same time, sequentially, before, after, or otherwise time-coordinated with gas generation. In some embodiments, a gas generator or a particle generator can be configured to provide a known bolus of the gas at the same time, sequentially, before, after, or otherwise time-coordinated with occupant monitoring, occupant number, or occupant activities.

### Particle Generators

Exemplary particle generators 102 are disclosed herein. Particle generator 102 generates an aerosol, in which it releases one or more test particles into indoor environment 10. In embodiments, the size of the test particles is above a first threshold of one or more of the particle mitigation systems in indoor environment 10. For example, if the filter of air purifier 60 has a filter profile to capture particles having a size greater than 0.3 microns, the test particle has a size of greater than 0.3 microns. In embodiments, an upper size limit on the test particles is 3.0 microns. In embodiments, the test particles have a size in the range of about 0.3 microns to about 3.0 microns.

In embodiments, one or more of the filters, such as in air purifier 60 or air handling system 20 are designed to have a filter profile that is most effective at filtering both large particles above a first threshold and small particles below a second threshold and least effective at particles between the first threshold and the second threshold. In embodiments, the first threshold is 1.0 microns and the second threshold is 0.1 microns. Thus, the hardest particles to filter are in the range of 0.1 microns to 1.0 microns. In embodiments, the test particles have a number mean particle diameter in the range of about 0.1 microns to about 1.0 microns. In embodiments, test particles have a number mean particle diameter in the range of about 0.2 microns to about 0.4 microns.

In embodiments, test particles have a number mean particle diameter in the range of about 0.03 microns to 0.3 microns. In embodiments, test particles have a number mean particle diameter in the range of about 0.06 microns to 0.3 microns. An advantage, among others, of test particles having a number mean particle diameter in the range of about 0.06 microns to 0.3 microns is that they are large enough to not agglomerate together when they collide resulting in larger particles, and a reduced number count. Another advantage, among others, of test particles having a number mean particle diameter in the range of about 0.06 microns to 0.3 microns is that they are small enough to remain suspended in the air of indoor environment 10 during the test period and not settle. A further advantage, of test particles having a number mean particle diameter in the range of about 0.06 microns to 0.3 microns is that their diffusion coefficient is low, limiting particle loss due to diffusion to wall surfaces, furniture or equipment ensuring and particles remain suspended as long as possible. The particle size ranges are understood to have the stated number mean particle diameter with a geometric standard deviation of between about 1.05 to about 2.5. Further, in embodiments, the particle size distribution of test particles is polydisperse with a log-normal distribution. In other embodiments, such as wherein the test particles are polystyrene latex spheres, the particle size distribution of test particles may be monodisperse.

Exemplary test particles include sodium chloride (NaCl), potassium chloride (KCl), Oleic Acid, Polystyrene Latex Spheres, Mineral Oil, Propylene Glycol, Saccharin (or other artificial sweeteners), Sucrose, Fructose, or other suitable test particles. In embodiments, wherein one or more light 80 are included in indoor environment 10, an exemplary test particle may be sensitive to the radiation emitted by light 80 and this sensitivity may be detected by one or more particles monitor 104. Exemplary test particles which are sensitive to 222 nm ultra-violet radiation include an oxidized iodide compound; Tetrabromomethane (CBr₄); non-toxic, non-peptide fluorophores; and/or fluorescent tagged proteins. Further, in embodiments, wherein the test particle is also naturally present in indoor environment 10, the test particles released into indoor environment 10 by particle generator 102 may have a fluorescence property, such as a fluorescent dye, that may be monitored by particle monitor 104 to distinguish the released test particles from the naturally present test particles. Exemplary test particles which have a fluorescence property that may be monitored by particle monitor 104 include Fluorescein dye.

In embodiments, a Förster resonant energy transfer (FRET) pair is used as a test particle. In a FRET, a pair of fluorophores may be bonded with peptide bonding and exhibit a first spectral response. UV radiation, such as 222 nm radiation emitted by one or more lights 80 in embodiments, will alter or break this peptide bonding which will cause a second spectral response. Thus, FRETs may be used to detect for test particles mitigated by air handling system 20 and/or air purifier 60 by monitoring for the first spectral response, such as a particular first wavelength with a particle monitor 104. Further, FRETs may be used to detect for test particles mitigated by light 80 by monitoring for the second spectral response, such as a particular second wavelength different from the first wavelength. Alternatively, indoor environment 10 may be recharged with FRETs back to the solenoid valve 352 level and light 80 activated which will alter a portion of the FRETs in indoor environment 10. By comparing the first spectral response of particle monitor 104 for a first charging where light 80 are not activated and the first spectral response of particle monitor 104 for a second charging where light 80 are activated, air characterization system 100 may determine an estimate of the number of particles mitigated by light 80.

In embodiments, a fluorescent protein is used as a test particle. In embodiments, fluorescent proteins may be photobleached to provide a detectable spectral response, such as a particular wavelength. In embodiments, fluorescent proteins may be photoconverted to provide a detectable spectral response, such as a particular wavelength.

Exemplary particle generators include aerosol generators such as a collison atomizer, an ultrasonic atomizer, a candle or combustion based source, and a vaporization device. An exemplary particle generator is disclosed in U.S. Pat. No. 8,544,826, the entire disclosure of which is incorporated herein by reference herein.

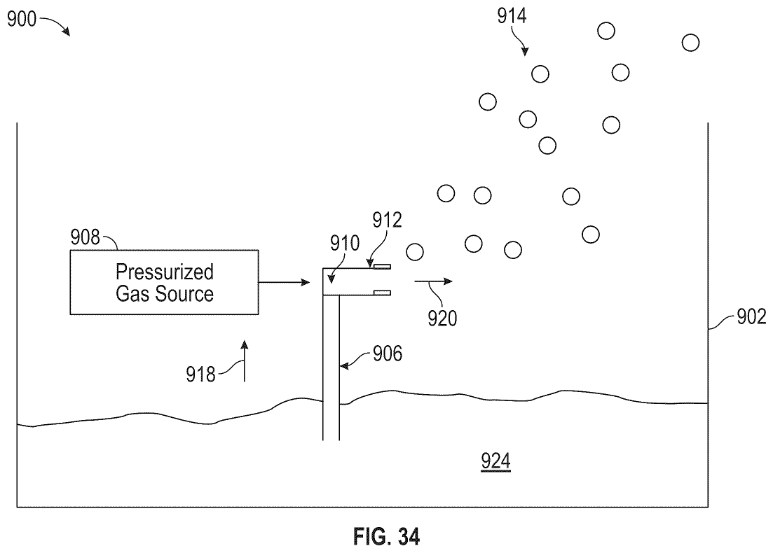

Referring to FIG. 34, an exemplary particle generator 900. Particle generator 900 includes a reservoir 902 having a solution 904 therein. A first fluid conduit 906 is positioned in solution 904 and acts as a syphon to pull the solution upwards in direction 918. First fluid conduit 906 is in fluid communication with a second fluid conduit 912 which acts as an expansion chamber. A pressurized gas source 908 is in fluid communication with second fluid conduit 912 through a nozzle 910. The pressurized gas flows through second fluid conduit 912 generally in direction 920 which causes a pressure drop at the top of first fluid conduit 906. This pressure drop acts to pull solution 904 upwards through first fluid conduit 906. When solution 904 reaches second fluid conduit 912, the gas flowing through second fluid conduit 912 cooperates with solution 904 to form an aerosol 914 which exits particle generator 900 and enters indoor environment 10.

In yet further examples, a particle generator can include a system configured to determine when occupants or occupant activity generate particles. In these examples, occupants or occupant activities are the particle source and the particle generator leverages such source to evaluate air quality. Human occupants and human activities can generate particles, and such particles can constitute test particles provided by a particle generator when such particle generation is suitably assessed in a controlled manner, and when such particles are of the type mitigated by the one or more particle mitigation systems. For example, candle burning, cooking, cleaning, and industrial processes can generate particles, which can be removed via various particle mitigation systems, such as mechanical filters. Other types of particles can also be generated by humans or human activities, such as sweating. Human-based particle generation can be useful as a test particle source when such generation can be controllably started and stopped, or when such generation can be identified as starting and identified as stopping. Sensors that detect occupancy (e.g., number of occupants) or occupant activity can be used to identified when particle generation starts or stops. An occupancy monitoring system can include such sensors to identify human particle generation activity suitable for equating to charging from a controlled release-based particle source. Even human-based activity that generates few particles can be sufficient for serving as a test particle provided that such particle charges the indoor environment to a level sufficiently above baseline, for example three-times above baseline. Some human-based activity, such as cooking, can generate large numbers of particles. For example, the monitoring system can identify entry of a single occupant entering the indoor environment and lighting a candle, which charges the room with particles. As another example, the monitoring system can identify when a stove turns on, which charges the room with particles. As another example, the monitoring system can identify when an occupant is exercising, which charges the room with particles. Indeed, addition of a human occupant to the indoor environment can serve as a particle source for particle generation. The monitoring system can also use such sensors to identify when particle generation activity terminates, which identifies when it is appropriate to measure decay and particle removal rate.

### Particle Monitors

Exemplary particle monitors 104 are disclosed herein. Particle monitors 104 analyze a portion of the air in indoor environment 10 and count the number of at least a first group particles therein, the first group of particles often being based on particle size.

A first exemplary particle monitor is a condensation particle counter which uses condensation vapor growth to enlarge particles, thereby effectively increasing the size particles to an easily detectable size (e.g., 0.10 μm particles may be enlarged to 5 μm). Growing particles larger enables a simple non-contact system, such as optical counter of the condensation particle counter, to detect and count the presence of smaller particles in the size range from about 0.10 μm to about 5 μm in diameter. An exemplary condensation particle counter is disclosed in U.S. Provisional Patent Application No. 63/443,834, filed Feb. 7, 2023, titled CONDENSATION NUCLEATION PARTICLE COUNTER, the entire disclosure of which is expressly incorporated by reference herein.

A second exemplary particle monitor is a laser particle counter. An exemplary laser particle counter is the SPS30 particulate matter sensor available from Senserion AG located at Laubisruetistrasse 50, 8712 Stafas, Switzerland. The SPS30 may measure particles up to 2.5 microns in size. Another exemplary laser particle counter is the OPS3300 available from TSI Incorporated located at 500 Cardigan Road in Shoreview, Minnesota 55126. The OPS3300 may measure particles in the range of 0.3 to 10 microns in size. A further exemplary laser particle counter is the PMS1003 available from Plantower Technology located at Three Floors, No. 3 Workshop, Yubo Science and Technology Park, Nanchang Economic and Technological Development Zone, Nanchang City, Jiangxi Province, China. The PMS1003 may measure particles in the range of 0.3 to 1.0 microns; 1.0 to 2.5 microns; and 2.5 to 10 microns. An exemplary laser based particle monitor is disclosed in US Published Patent Application No. US20220042900A1, titled PARTICULATE MATTER SENSOR DEVICE, the entire disclosure of which is expressly incorporated herein by reference. Additional laser based particle counters include self-mixing interferometry optical particle counters.

A second exemplary particle monitor is a particle surface area diffusion charger device. An exemplary diffusion charger device is the DC 2200CE diffusion charger available from EcoChem Analytics located at 202 Reynolds in League City, Texas 77573. The DC 2200CE may measure particles up to 10 microns in size. Another exemplary diffusion charger device is the TSI 3550 available from TSI Incorporated located at 500 Cardigan Road in Shoreview, Minnesota 55126. The TSI 3550 may measure particles in the range of 0.01 to 1.0 microns in size. Exemplary charger devices are disclosed in U.S. Pat. Nos. 6,544,484 and 7,812,306, the entire disclosures of which are expressly incorporated herein by reference.

### Gas Generators

Exemplary gas generators 106 are disclosed herein. Gas generator 106 releases one or more gases or “test gases” into indoor environment 10 that are monitored by the gas monitor. In various embodiments, the test gas is filtered either during bottling or release from a container. In other embodiments, test gas is produced by an occupant or occupant activity and the amount or rate of gas generated is estimated or determined from use of a sensor that detects occupancy and occupant activity.

In embodiments, suitable test gases are not filtered by mechanical filtration. In embodiments, test gases should be safe for humans and are generally neutrally buoyant with the air of indoor environment 10. Exemplary test gases include carbon dioxide (CO₂); an exemplary volatile organic compound (VOC), such as methane, butane propoane, 2-proponal, Ethanol, formaldehyde, ammonia; water vapor (H₂O); radon; oxygen (¹⁸O₂); ozone (O₃); heavy water (D₂O); Nobile gases such helium (He₂), Xenon, and Argon; sulphur hexafluoride (SF₆); Dimethydinitrobutane; Tetrafluoroethane; Tetrafluoropropene; and other suitable test gases. In various further embodiments, the gas is a gas that is not removed via a mechanical filtration process, physicochemical processes (e.g., scrubbing), or light-mediated removal process (e.g., UVC). In other embodiments, the gas is a gas that is removed via at least one of a mechanical filtration process, physicochemical processes (e.g., scrubbing), or light-mediated removal process (e.g., UVC). In various further embodiments, the gas is a gas that is not removed via any non-ventilation portion of a particle mitigation systems, such as mechanical filtration process, physicochemical processes (e.g., scrubbing), light-mediated removal process (e.g., UVC).

In yet further embodiments, two or more test gases are utilized each having different interactions with one or more of the various particle mitigation systems, thus enabling disambiguation of the performance of discrete portions of the particle mitigation systems. For example, carbon dioxide and isopropanol can be used together to determine the performance of activated carbon since CO2 is does not react with the activated carbon and is only reduced by mixing with fresh air, while isopropanol is captured by the activated carbon. In various embodiments, the two or more test gases are generated from separate sources. In other embodiments, the two or more test gases are generated from the same source.

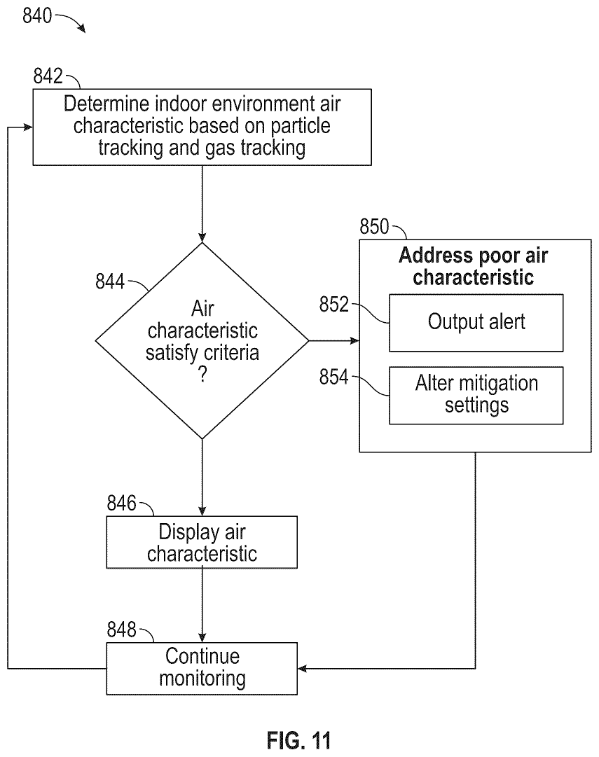

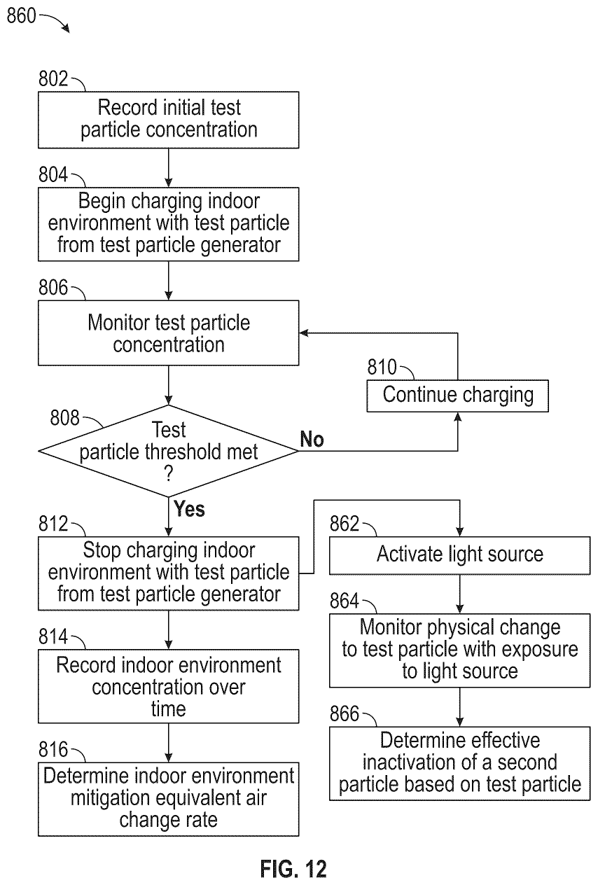

A first exemplary gas generator 300 is shown in FIGS. 11 and 12. Gas generator 300 includes a gas source 302. An exemplary gas source is a container 304 of pressurized gas having an outlet 306 in fluid communication with a first valve, illustratively a first solenoid valve 308. Solenoid valve 308 is also in fluid communication with a chamber 310. Chamber 310 is sized to receive a measured quantity of the test gas from container 304 through first solenoid valve 308.

A sensor 312 monitors chamber 310. Exemplary sensors include a pressure sensor and a radiation sensor if the test gas has a radioactive characteristic. Electronic controller 130 monitors the sensed characteristic and opens and closes first solenoid valve 308 based thereon.

A second valve, illustratively a second solenoid valve 320 is also in fluid communication with chamber 310 and a vent 322 which is in fluid communication with indoor environment 10. When second solenoid valve 320 is opened, the test gas is released from chamber 310 into indoor environment 10. Electronic controller 130 based on a stored value of the volume of chamber 310, the sensed value from sensor 312, such as pressure, and the concentration of the test gas in gas source 302 is able to quantify a number of test gas particles released into indoor environment 10. In embodiments, a single filling and release of particles from chamber 310 is a desired dose or charge of test gas into indoor environment 10. In embodiments, multiple fillings and releases of test gas particles from chamber 310 is a desired dose or charge of test gas into indoor environment 10.

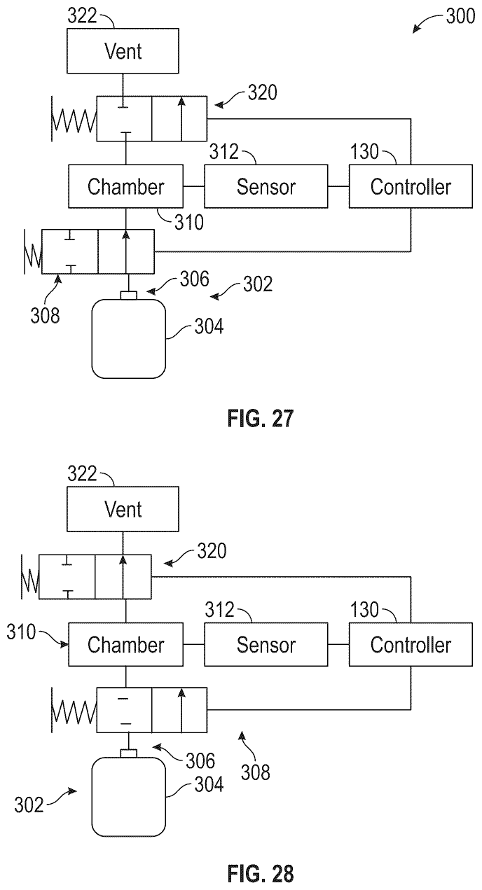

Referring to FIG. 27, electronic controller 130 has first solenoid valve 308 in an open configuration wherein gas flows from container 304 into chamber 310 and second solenoid valve 320 in a closed configuration wherein gas is prevented from flowing from chamber 310 to vent 322 into indoor environment 10. the current dose based on the pressure and known volume of the measured chamber. Referring to FIG. 28, electronic controller 130 has first solenoid valve 308 in a closed configuration to prevent additional gas from flowing from container 304 into chamber 310 and second solenoid valve 320 in an open configuration wherein gas is released from chamber 310 to vent 322 and into indoor environment 10. At all times, one or both of first solenoid valve 308 and second solenoid valve 320 are in a closed configuration which reduces the chance of additional test gas entering indoor environment 10. In embodiments, the de-energized state of each of first solenoid valve 308 and second solenoid valve 320 is the closed configuration and electronic controller 130 must energize the desired one of first solenoid valve 308 and chamber 310 to open the valve. Thus, in the case of power failure the default configuration for first exemplary gas generator 300 is both of first solenoid valve 308 and second solenoid valve 320 in the closed configuration.

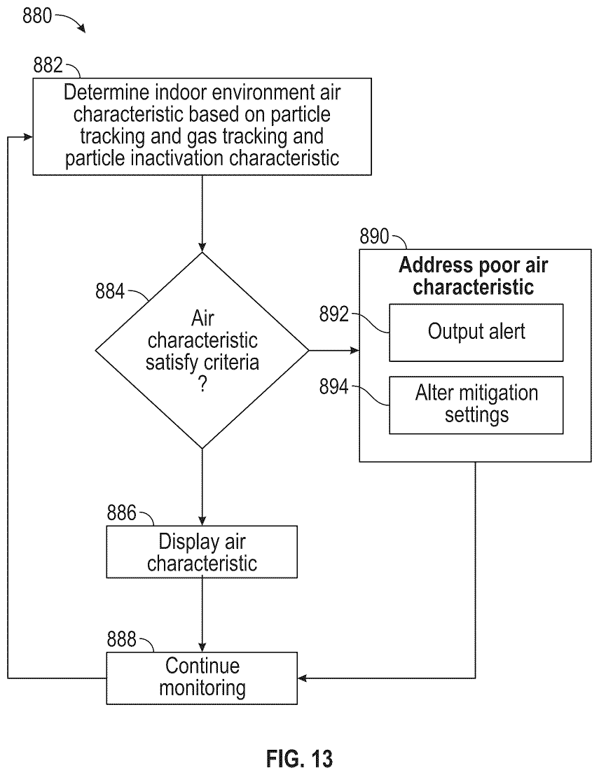

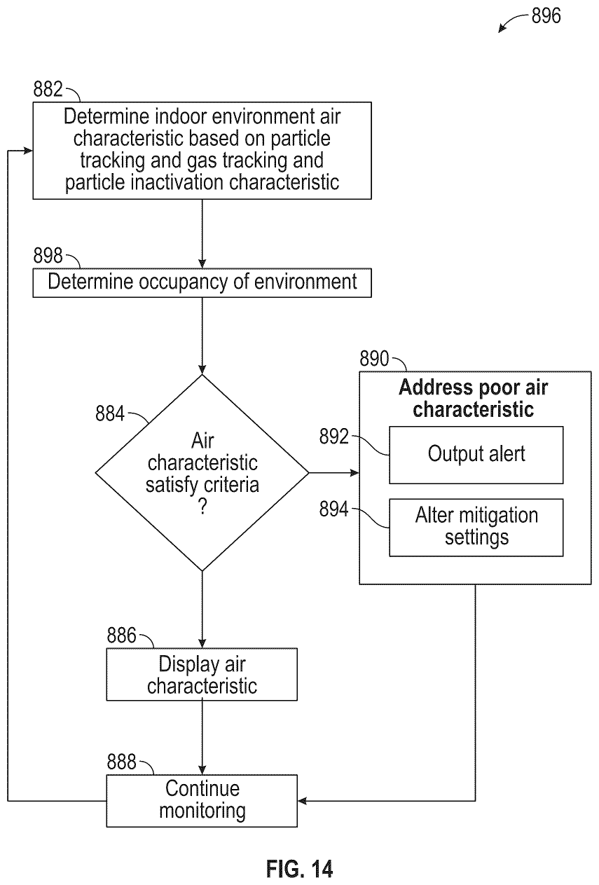

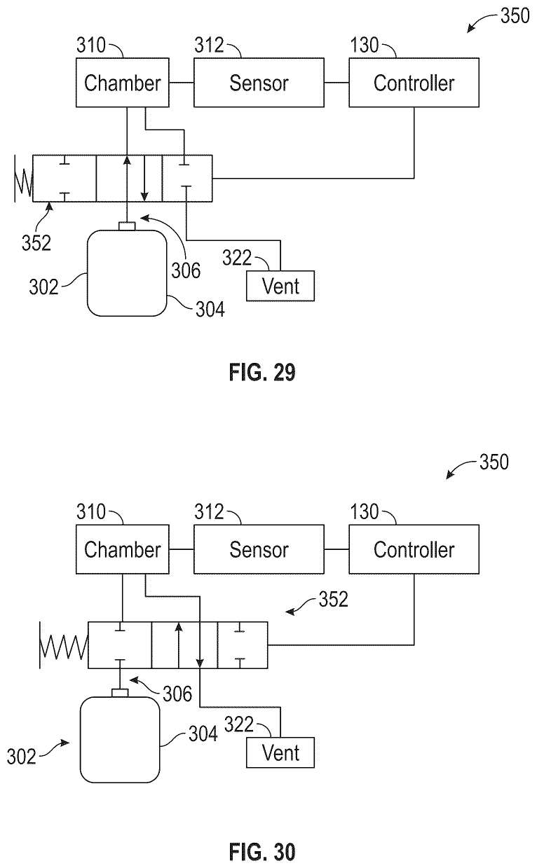

A second exemplary gas generator 350 is shown in FIGS. 13 and 14. Gas generator 350 is generally similar to first exemplary gas generator 300 with first solenoid valve 308 and second solenoid valve 320 replaced with a single valve, illustratively solenoid valve 352. Referring to FIG. 29, solenoid valve 352 is in a first configuration, an energized configuration, wherein gas may flow from container 304 to chamber 310, but not from chamber 310 to vent 322. Referring to FIG. 30, solenoid valve 352 is in a second configuration, a deenergized configuration, wherein gas may flow from chamber 310 to vent 322, but not from container 304 to chamber 310.

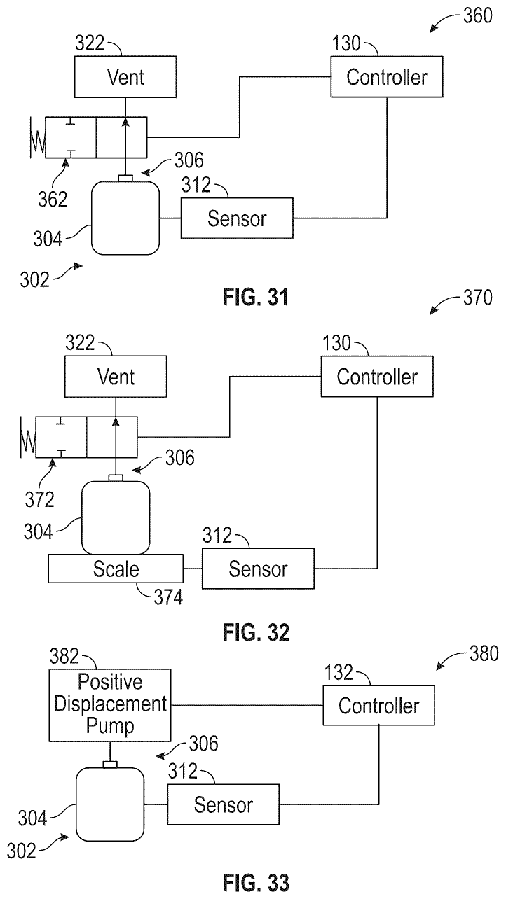

A third exemplary gas generator 360 is shown in FIG. 31. Gas generator 360 includes container 304 coupled directly to vent 322 through a first valve, illustratively a solenoid valve 362. Sensor 312 is a pressure sensor which monitors a pressure of the gas within container 304. Electronic controller 130 by knowing a time duration that solenoid valve 362 is open, as shown in FIG. 31, the pressure of the gas in container 304, if the gas in container 304 is a mixture (the concentration of the target gas for testing), and the size of the orifice of outlet 306 may determine the amount of target gas released from container 304 into indoor environment 10.

A fourth exemplary gas generator 370 is shown in FIG. 32. Gas generator 370 includes container 304 coupled directly to vent 322 through a first valve, illustratively a solenoid valve 372. Sensor 312 is a weight sensor which monitors a scale 374 that supports container 304. Electronic controller 130 by knowing a weight of container 304 prior to and post release of a test gas time duration that solenoid valve 362 is open, if the gas in container 304 is a mixture (the concentration of the target gas for testing), and the ideal gas law may determine the volume of target gas released from container 304 into indoor environment 10.

A fifth exemplary gas generator 380 is shown in FIG. 33. Gas generator 380 includes container 304 coupled directly to a positive displacement pump 382 which repeatedly encloses a fixed volume of gas from container 304 subsequently releasing that fixed volume of gas into indoor environment 10. A sensor 312 is provided to monitor a pressure within container 304 to verify the presence of a suitable amount of test gas in container 304.

In yet further examples, a gas generator can include a system configured to determine when occupants or occupant activity generate gas. In these examples, occupants or occupant activities are the gas source, which the gas generator utilizes to evaluate air quality, ventilation, or gas removal rates. Human occupants and human activities can generate gas (e.g., CO2), and such gas can constitute test gas provided by a gas generator when such gas generation is suitably assessed in a controlled manner. For example, occupants generate CO2 via respiration and the CO2 generation rate can be estimated based on number of occupants and occupant activity (e.g., occupants at resting, walking, or engaging in vigorous exercising). Use of occupant sensors that monitor occupant number, occupant activity, or occupant movement can be used to determine CO2 generation rate. Such sensors include CO2 sensors, which directly measure CO2 levels in the indoor space: changes in CO2 correspond to changes in number of occupants or changes in occupant activity. In combination with other sensors, a complete picture of CO2 generation due to occupants and their activities is possible. Other sensors can also indirectly determination of CO2 generation rate via detecting number of occupants or detecting type of occupant activity and estimating the amount of CO2 generation occurring in the given space. Other types of gas can also be generated by humans or human activities, such as organic materials from human bodies or cleaning activities. Human-based gas generation, particle CO2, can be useful as a test gas source when such generation can be controllably started and stopped, or when such generation can be identified as starting and identified as stopping. Sensors that detect occupancy (e.g., number of occupants) or occupant activity can also be used to identified when gas generation starts or stops, for example when an occupant enters or leaves the space. An occupancy monitoring system can thus identify a time frame for equating human gas generation to charging from a controlled release-based gas source. Likewise, occupancy monitoring system can identify a time frame suitable for equating to a decay period or a identify a termination of gas generating event, for example when one or more gas generating person leaves the space, which are useful for determining gas removal rate or ventilation. Even human-based activity that generates little gas can be sufficient for serving as a test gas provided that such gas charges the indoor environment to a level sufficiently above baseline, for example three-times above baseline.

### Gas Monitors

Exemplary gas monitors 104 are disclosed herein. Exemplary gas monitors 104 include dual and single channel NDIR carbon dioxide sensors. An exemplary carbon dioxide sensor is the Sensirion SCD30 available from Sensirion AG located at Laubisruetistrasse 50, 8712 Stäfa, Switzerland. Another exemplary carbon dioxide sensor is disclosed in U.S. Pat. No. 5,444,249, the entire disclosure of which is expressly incorporated by reference herein. Another example of a CO2 sensor is a Sunrise CO2 sensor from Senseair in Delsbo, Sweden. Further exemplary particle monitor 104 for methane include catalytic bead combustible sensors or Infared thermopile detectors. An exemplary catalytic bead combustible sensor is the Alphasense CH-A3 available from Alphasense located at 24 Union Square East, 5th Floor in New York, NY 10003. An exemplary Infared thermopile detector is the Alphasense IRM-AT. Additional exemplary particle monitor 104 for hydrogen sulfide is a metal oxide semiconductor or electrochemical sensor such as the AlphaSense H2S-BH. Further exemplary particle monitor 104 for water vapor include polyamide film type humidity sensors or thin film capacitive humidity sensor. An exemplary polyamide film type humidity sensor is the Silcon Labs Si7005 available from Silicon Labs located at 400 West Cesar Chavez in Austin, Texas 78701. An exemplary or thin film capacitive humidity sensorfis the Vaisala HMP9 available from Vaisala OYJ located at Vanha Nurmijarventie 21, 01670 Vantaa, Finland. Additional particle monitor 104 for VOCs include electrochemical VOC sensors. Exemplary electrochemical VOC sensors include the Alphasense VOC-A4 and the Alphasense VOC-B4. Further particle monitor 104 for sulfur hexafluoride (SF6) include photoionization detectors and nondispersive infrared sensors. An exemplary photoionization detector is the Ion Science MINIPID2HS available from lon Science Inc. located at 4153 Bluebonnet Dr. in Stafford, Texas 77477. An exemplary nondispersive infrared sensor is the Nano Enviroment Technology IREF-P-32 available from Nano Environment Technology located at Via Campania 5, 20006 Pregnana Milanese in Milano, Italy. Additional exemplary gas monitor 104 for ozone include oxidizing gas sensors. An exemplary oxidizing gas sensor is the Alphasense OX-B431. Further exemplary particle monitor 104 for noble gases include compact mass spectrometer or gas chromatography. An exemplary device is the Vernier Mini GC Plus available from Vernier Science Education located at 13979 SW Millikan Way in Beaverton, Oregon 97005.

### Occupant Sensors

The systems and methods described herein can utilize occupancy sensors and occupant activity sensors to utilize occupants and occupant activities as a source of particle or gas generation. While device-based gas and particle sources can be readily controlled such that charging can be started and stopped at will, passive sources of gas and particles are monitored until a suitable time when passive activity can be recognized as a charging, steady state, or decay period suitable for recording data points for determining gas or particle removal. While concentration and other air characteristics alone can be sufficient for a user or computer to determine whether the environment is in a charging state, steady state, or decay period, it can be additional useful to use occupant monitoring to determine when changes in occupancy or occupant activities suggests a charging event, a steady state period, or a decay period. In some embodiments, it may be sufficient to rely entirely on occupant sensors for determining initiation or termination of particle or gas generating activity. An occupancy sensor can include a sensor configured to assess or measure an amount of occupants in a space. Typically, the sensor is configured to determine a number of occupants (e.g., people) in a space. The occupancy sensor can be a CO2 sensor, a microphone, a camera, a mmWave sensor, a sensor, a wi-fi sensor configured to detect the presence of people, an infrared sensor, or a sensor detected to detect the presence of discrete personal electronic devices (e.g., phones) so as to detect or estimate the number of occupants. The occupancy sensor system or a component thereof may be configured with on-board, or remote, machine learning technology, which can be useful for utilizing general-type sensors for assessing, categorizing, or measuring occupancy, breaths, and activity.

The occupant activity sensor can include a sensor configured to assess, categorize, or measure activity level or type of activity of occupants in the space. Typically, the sensor is configured to determine activities associated with respiration, such as working out (higher respiration) or watching a movie (lower respiration), or associated with projection of aerosol, such as loud talking in a crowded bar (higher projection) or quiet reading in a library (lower projection). The occupant activity sensor, which configured as an activity sensor, can be a CO2 sensor, a microphone, a camera, a light sensor, mmWave sensor, or an infrared sensor. The occupant activity sensor can include a sensor configured to assess, categorize, or measure breathing, breath number, breath rate, or breath volume. Typically, the sensor is configured to detect breathing in the context of determining increased emission of pathogen (which is useful for the source generation model) and increased inhalation of pathogen (which can be used to as transmission risk can be determined based on inhaled dose). The occupant activity sensor, which configured as a breathing sensor, can be a CO2 sensor, a mmWave sensor, or an infrared sensor. The occupant activity sensor can also include a sensor configured to assess, categorize, or measure heart beats.

The occupancy sensor system can include a mmWave sensor that utilizes mmWave radar. Millimeter wave (mmWave) radar is a type of radar that uses radio waves in the millimeter range of the electromagnetic spectrum to detect objects and their characteristics. Millimeter wave radar operates at frequencies typically in the range of 24 GHz to 300 GHz. At these frequencies, the wavelengths are in the millimeter scale, which allows the radar to detect small movements and provide high-resolution images. Additionally, it has the ability to penetrate certain materials, such as clothing, which makes it useful for detecting objects or people without direct line-of-sight. Millimeter wave radar can detect the presence of people in a given area, even through walls or obstacles, and by analyzing the radar returns and using signal processing algorithms, it can discern multiple entities and count the number of people present in a space. Additionally, mmWave radar can detect the chest movements caused by breathing due to its high sensitivity to movement. By measuring the periodic motion from the chest wall, one then can calculate the breathing rate. Similar to breathing rate monitoring, the subtle chest vibrations due to the heartbeat can also be detected by mmWave radar, allowing for non-contact heart rate monitoring.

The occupancy sensor system can include a microphone. A microphone serves as an acoustic sensor that can be pivotal in occupancy detection systems. By monitoring ambient sound levels, it can discern the presence of individuals through detection of noise signatures typical of human activity, such as footsteps, door operations, or shifting furniture. This capability is enhanced by signal processing algorithms that analyze the sound spectrum for patterns associated with occupancy, differentiating them from the baseline environmental noise. The presence of intermittent or continuous sounds within the human auditory range suggests occupancy, and can be quantified to determine the likelihood of a room being occupied. The sensor's output, an array of audio signals, is systematically evaluated in real-time to ascertain the occupancy status without the need for visual cues. Furthermore, the analysis of audio signals extends to identifying specific activities taking place within a space. Distinctive sound patterns, such as the rhythmic keystrokes on a computer keyboard or the consistent hum of kitchen appliances, enable the determination of different activities. The system can be trained to recognize these patterns using machine learning techniques and classify them into categories such as working, cleaning, or cooking. Speech detection and analysis through frequency and amplitude modulation patterns can reveal communication dynamics, while the detection of regular acoustic events, such as the sound of writing instruments on paper, can suggest studying or administrative work. By applying advanced acoustic analysis and classification algorithms, the microphone sensor can effectively interpret the soundscape to provide detailed insights into the activities being conducted within a monitored room.

The occupancy sensor system can include a light sensor, such as an ambient light sensor. Ambient light sensors can be useful for occupancy detection. Ambient light sensors measure the intensity of light within an environment. In the context of occupancy detection, they can be used to determine changes in light conditions that may correlate with the presence or absence of people. Ambient light sensors can be configured to detect abrupt variations in light levels, such as those caused by a person entering a room and casting a shadow, or the act of turning on a light source. They can also monitor gradual changes in ambient light that align with expected occupancy patterns during different times of the day. Sophisticated algorithms can analyze the data collected to distinguish between natural fluctuations in light levels, like those caused by passing clouds, and those alterations likely induced by human activity. The integration of an ambient light sensor with a microphone sensor can significantly enhance the granularity and accuracy of activity detection within a space. The ambient light sensor, by quantifying the intensity and spectral composition of environmental lighting, can detect occupancy-related light events, such as the opening of a door, which may cause a sharp increase in light level, or the activation of a display screen, which alters the light spectrum. In tandem, the microphone sensor acquires acoustic data, which is processed to identify sound patterns and signatures associated with human activities. By employing machine learning algorithms, the system can analyze and correlate the temporal relationship between light-based events and acoustic signals, allowing for a more nuanced interpretation of activity. For instance, a sudden increase in light detected by the ambient light sensor, coinciding with the characteristic sound of a door opening captured by the microphone, can suggest entry into the space. If this event is followed by the steady-state lighting typically associated with occupancy and a consistent acoustic background of keyboard typing or low-level conversation, the system can infer that the space is being used for work-related activities. Conversely, fluctuations in lighting accompanied by varied sound patterns could indicate a more dynamic setting, such as a social gathering or a collaborative workspace. The combined data streams offer a multidimensional perspective that is less prone to false positives than either sensor would be in isolation, enhancing the system's ability to detect and categorize activities based on the specific light and sound signatures present. This multimodal approach not only refines occupancy detection but also the determination of activities that could be occurring in the space.

The occupancy sensor system can include a CO2 sensor. The concentration of CO2 within an indoor environment is a reliable indicator of occupancy because humans exhale carbon dioxide as part of the respiratory process. As outlined in the following journal article by Davide Call et al., a CO2-based occupancy detection algorithm can be implemented to estimate the number of people in a space by measuring the levels of CO2 present. (Davide Cali, Peter Matthes, Kristian Huchtemann, Rita Streblow, Dirk Müller, CO2 based occupancy detection algorithm: Experimental analysis and validation for office and residential buildings, Building and Environment, Volume 86, 2015, Pages 39-49). This method capitalizes on the predictable increase in CO2 concentration that correlates with human presence and activity. For instance, in an office setting, the CO2 levels would typically rise during the morning as employees arrive and start their workday, reach a plateau during the steady state of occupancy throughout the day, and then gradually decrease after hours as people leave the space. By monitoring these variations, the algorithm can estimate the number of occupants with reasonable accuracy. The study by Calì and colleagues further validates this approach through experimental analysis in both office and residential buildings. They demonstrate that the CO2 concentration not only reflects the presence of people but can also be used to deduce occupancy patterns over time. In a residential setting, for example, sharp increases in CO2 levels might be observed during morning and evening hours, corresponding with the times people are generally at home and active. Conversely, lower levels during the day can indicate the house is unoccupied. By establishing a baseline and accounting for variables such as ventilation rates and the volume of the space, the CO2 detection algorithm can effectively determine occupancy rates, providing essential occupancy and inferred activity information for a risk transmission model.

The occupancy sensor system can include a camera-based sensor. While various conventional camera-based sensors can be suitable for determining occupancy number, occupant activity, and occupant breathing, a particularly useful camera are smart cameras incorporating machine vision technology, processed locally or remotely, that provide face recognition, object tracking, object recognition, line tracking, color recognition, tag recognition, and object classification. For example, machine vision cameras can recognize people, faces, and objects. Such cameras can be readily configured for counting the number of occupants in a space. One example commercially available camera with built-in machine learning technology is a HeskyLens AI Camera available from Zhiwei Robotics Corp (Shanghi, China).

Additionally, occupant monitoring and CO2 monitoring can be useful to determine when ventilation or other backgrounds may be fluctuating or inconsistent, for the purposes of particle monitoring and analysis. For example, in some embodiments, if the background ventilation rate (ACH) is consistent as determined by a gas (e.g., CO2 monitor), and yet it can be inferred that an occupant did something or stopped doing something that generates particles, then it can be possible to use occupant monitoring to identify when to measure the particle removal rate from the decay curve based on an initiation of occupant activity and subtract the background out.

### System Operation

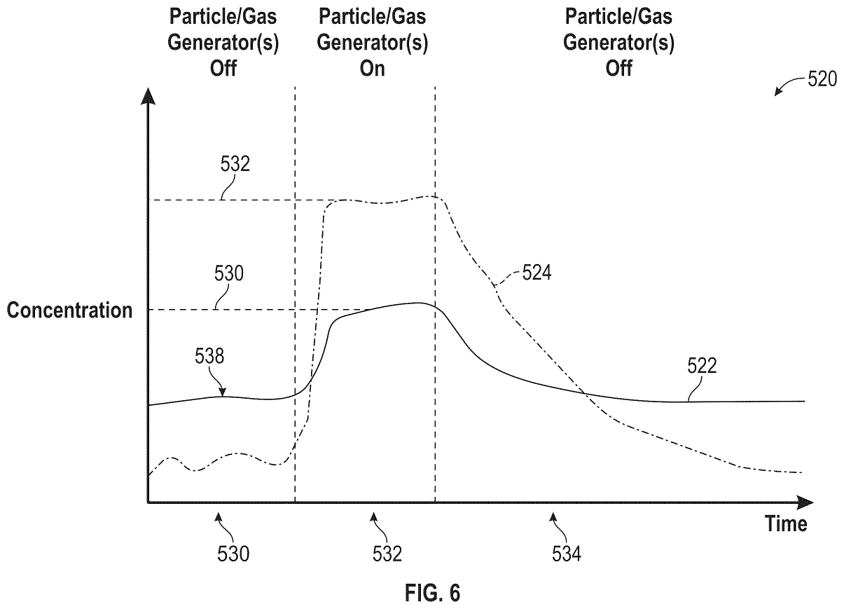

Referring to FIG. 6, a graph 520 illustrating a concentration over time of a test gas in indoor environment 10, represented by test gas curve 522, and a concentration over time of a test particle, represented by test particle curve 524, is shown. Graph 520 is divided into three regions: a pre-charge region 530, a charge region 532, and a post-charge region 534. In pre-charge region 530, each of test gas curve 522 and test particle curve 524 are generally steady-state and both particle generator 102 and gas generator 106 are not actively introducing respective test particles and test gases into the indoor environment 10. In charge region 532, both of test gas curve 522 and test particle curve 524 are raised to a higher concentration level than steady-state due to each of particle generator 102 and gas generator 106 actively introducing respective test particles and test gases into the indoor environment. In post-charge region 534, both of test gas curve 522 and test particle curve 524 begin to decay due to each of particle generator 102 and gas generator 106 being deactivated and not actively introducing respective test particles and test gases into the indoor environment 10. While each of particle generator 102 and gas generator 106 are shown as being activated in the same charge region 532, in embodiments, each of particle generator 102 and gas generator 106 is activated in its own charge region 532. For example, gas generator 106 may be activated in a first charge region and particle generator 102 activated in a second charge region subsequent to the first charge region. In examples, the second charge region may at least partially overlap with the first charge region resulting in each of gas generator 106 and particle generator 102 being active at the same time.

In embodiments, the presence of test particles from particle generator 102 allows electronic controller 130 to quantify the effectiveness of one or more of the particle mitigation systems, such as mechanical filters 30 of air handling system 20, air purifier 60, and light 80 and the presence of test gas from gas generator 106 allows electronic controller 130 to quantify the effectiveness of air handling system 20 and the introduction of outdoor air through third fluid conduit 40.

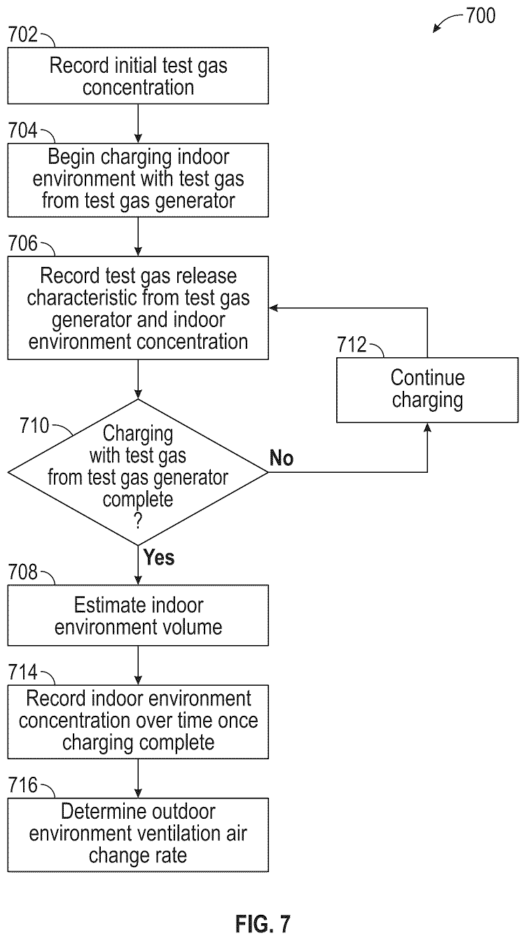

Referring to FIG. 7, an exemplary processing sequence 700 of electronic controller 130 is shown. An initial concentration of the test gas is recorded, as represented by block 702. In embodiments, one or more sensors 110 of gas monitor 108 records a signal representative of the concentration of the test gas just prior to charge region 532 in graph 520. Indoor environment is charged with test gas from gas generator 106, as represented by block 704. Electronic controller 130 records the amount of test gas released from gas generator 106 into indoor environment 10, as represented by block 706. If multiple charges are needed, electronic controller 130 loops back and requests that gas generator 106 release further charges of test gas to indoor environment 10, as represented by blocks 710 and 712. Blocks 702, 704, 706, 710, and 712 are collectively referred to as the charging portion of processing sequence 700 while the remaining blocks of processing sequence 700 are collectively referred to as the monitoring portion of processing sequence 700. Once a threshold amount 530 is achieved, in embodiments, electronic controller 130 estimates a volume of indoor environment 10. In an example, the volume of indoor environment 10 may be estimated from equation 1:

$$INDOOR ENVIRONMENT VOLUME = \frac{NUMBER OF TEST IN CHARGE}{{C}_{CHARGE} - {C}_{INTIAL}} \qquad ( 1 )$$

In embodiments, the volume of indoor environment 10 is input with input devices 140. In this example, the entered volume of the room provides an indication based on the concentrations recorded by gas monitor 108 how well mixed the air in indoor environment 10 is. Returning to processing sequence 700, electronic controller 130 records the concentration of test gas once charging is complete with gas monitor 108, as represented by block 714.

Electronic controller 130 then determines the outdoor environment ventilation air change rate (ACH) for air handling system 20 of indoor environment 10, as represented by block 716. In embodiments, electronic controller 130 monitors with gas monitor 108 the concentration of the test gas in indoor environment 10 over time and logs those readings. In one example, the outdoor environment ventilation air change rate (ACH) is determined based on equation 2:

$$ACH = \frac{- 1 * ln ( \frac{{C}_{end} - {C}_{ambient}}{{C}_{start} - {C}_{ambient}} )}{{t}_{end} - {t}_{start}} \qquad ( 2 )$$

wherein Cₛₜₐᵣₜ is the initial concentration of the test gas at time tₛₜₐᵣₜ, C_{end} is the ending concentration of the test gas at time tend, and C_{ambient} is the background level of the test gas in the outdoor air being drawn in through third fluid conduit 40 of air handling system 20. In the case of CO2 being the test particle, the outdoor background level of CO2 is typically about 300-400 parts per million (ppm).

In embodiments, the outdoor environment ventilation air change rate (ACH) for air handling system 20 of indoor environment may be determined by fitting the data points of curve 522 to equation 3:

$$C = ( \frac{D}{{Q}_{A}} + {C}_{B} ) ( 1 - {e}^{- ACH * t} ) + {C}_{I} {e}^{- ACH * t} \qquad ( 3 )$$

wherein C is the concentration of the test gas in indoor environment 10 (moles/m³ or PPM), D is the release rate of the test gas in indoor environment 10 (mole/sec) of gas generator 106, Q_{A} is the air volume supply rate of test gas generator 106 (m³/sec), C_{B} is the background concentration of the test gas in outdoor environment 38 (moles/m³ or PPM), ACH is the outdoor environment ventilation air change rate which is equal to Q_{R}/V (Q_{R} is the air volume supply rate to indoor environment 10 by air handling system 20 (m³/sec) and V is the volume of indoor environment 10 (m³)), t is the elapsed time (sec), and C, is the initial concentration of test gas in indoor environment 10 (moles/m³ or PPM). Each of D, Q_{A}, and C_{B} are provided as inputs to air characterization system 100 through input devices 140 and CI is recorded by air characterization system 100. Each of C and t are ordered pairs of the data 138 stored in memory 134 that forms test gas curve 522. In embodiments, air characterization system 100 selects a value of ACH by fitting the ordered pairs of data 138 to equation 3 and selecting the value of ACH that reduces the error between the respective ordered pairs and equation 3. In embodiments, each ordered pair of C and t are used to compute a value of ACH and an average of the ACH values is reported by air characterization system 100 as the ACH value of air handling system 20. If air characterization system 100 receives QR for air handling system 20 as an input, air characterization system 100 based on the determined ACH may determine a value for the volume V of indoor environment 10.

In embodiments, wherein the test gas is carbon dioxide, air characterization system 100 compares the generally steady state value 538 of the test gas in indoor environment 10 for pre-charge region 530 of graph 520 to an expected value stored in associated memory 134 for an unoccupied indoor environment 10 having air handling system 20 running. If the steady state value 538 is within a first threshold of the expected value stored in associated memory 134, then indoor environment 10 is determined to be unoccupied. If the steady state value 538 exceeds the expected value by more than the first threshold, air characterization system 100 determines a number of individuals in indoor environment 10 based on the steady state value 538 and one or more characteristics of indoor environment 10 inputted to air characterization system 100 with input devices 140. Exemplary characteristics of indoor environment 10 include a purpose of indoor environment 10. The amount of carbon dioxide exhaled by people in a given time period is largely dependent on the activity in indoor environment 10. A fitness center with people exercising would have a different carbon dioxide production rate than a classroom, a movie theater, or a restaurant wherein people are generally seated. By knowing the steady state value of 538 and the current settings of air handling system 20, either by direct monitoring or through input devices 140, air characterization system 100 may still determine the ACH of air handling system 20. In one example, air characterization system 100 alters the current setting of air handling system 20, either directly or by instructing an operator with output devices 142 to alter the current settings of air handling system 20, monitors the change in carbon dioxide based on a knowledge of the expected carbon dioxide production from the people in indoor environment 10 and the decay rate of carbon dioxide monitored with gas monitor 108 over time. In this way, the people in indoor environment 10 may be considered gas generators 106. Examples of settings that can be modified include amount of ventilation, fan speed, filters, air handler settings, and germicidal light. For example, ventilation can be lowered to an amount that results in a satisfied air quality threshold (e.g., CADR, CADR per occupant, ACH+MEACH, infection risk, etc.) while resultingly reducing indoor air heating or cooling, noise, component degradation, fuel consumption, electricity consumption, or costs. As another example, ventilation can be raised when doing so would satisfy a target threshold level of air quality together when considering the performance of the particle mitigation system (e.g., CADR, CADR per occupant, ACH+MEACH, infection risk, etc.). As another example, ventilation can be raised when doing so would satisfy a target threshold level of performance of the one or more particle mitigation systems, or a component thereof.

In embodiments, the test gas is not carbon dioxide nor another gas exhaled by people. This allows air characterization system 100 to function regardless of whether indoor environment 10 is occupied or unoccupied.

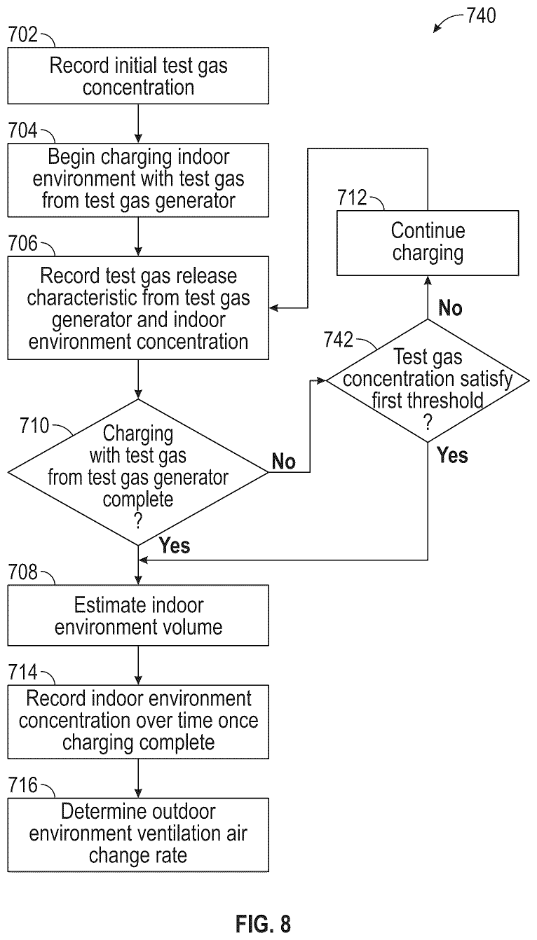

Referring to FIG. 8, another exemplary processing sequence 740 of electronic controller 130 is shown which is generally the same as processing sequence 700. Blocks 702, 704, 706, 710, 742, and 712 are collectively referred to as the charging portion of processing sequence 740 while the remaining blocks of processing sequence 700 are collectively referred to as the monitoring portion of processing sequence 740. In processing sequence 740, electronic controller 130 determines if charging is complete by recording the amount of test gas released from gas generator 106 into indoor environment 10, as represented by block 706, and comparing the amount to a threshold amount 530 (see FIG. 6), as represented by block 742. If the threshold amount 530 is achieved in a single charge, the processing sequence continues on to bock 708. If multiple charges are needed to achieve the threshold amount 530, electronic controller 130 loops back and requests that gas generator 106 release further charges of test gas to indoor environment 10, as represented by blocks 710 and 712.

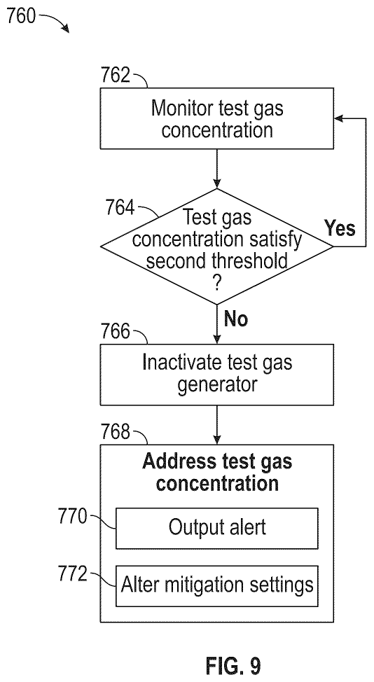

Referring to FIG. 9, a further exemplary processing sequence 760 of electronic controller 130 is shown. Air characterization system 100 monitors the test gas concentration in indoor environment 10, as represented by block 762. In embodiments, electronic controller 130 monitors the test gas concentration of indoor environment 10 with gas generator 106 continuously. In embodiments, electronic controller 130 only monitors the test gas concentration of indoor environment 10 with gas generator 106 during the charging portion of processing sequence 700 or processing sequence 740. In embodiments, electronic controller 130 only monitors the test gas concentration of indoor environment 10 with gas generator 106 during the monitoring portion of processing sequence 700 or processing sequence 740. In embodiments, electronic controller 130 monitors the test gas concentration of indoor environment 10 with gas generator 106 during both the charging portion of processing sequence 700 or processing sequence 740 and the monitoring portion of processing sequence 700 or processing sequence 740.

The measured test gas concentration is compared to a second threshold to see if the measured concentration satisfies the second threshold, as represented by block 764. In embodiments, the measured concentration satisfies the second threshold if it is below the second threshold. If the measured test gas satisfies the second threshold, electronic controller 130 returns to continuing to monitor the test gas concentration of indoor environment 10.

If the measured test gas concentration does not satisfy the threshold, electronic controller 130 inactivates gas generator 106 if activated, as represented by block 766. In embodiments, electronic controller 130 may take additional steps to address the measured test gas concentration not satisfying the second threshold as represented by block 768. In embodiments, electronic controller 130 may output an alert with output devices 142. Exemplary alerts include activating a visual indicator, such as flashing a light or displaying a message on a screen, activating an audio indicator, such as emitting an audible tone or message, and/or sending a message to a remote device with an exemplary communication module.

Further, in embodiments, electronic controller 130 may modify one or mitigation settings for the particle mitigation systems of indoor environment 10, as represented by block 772. In embodiments, electronic controller 130 may send a message to air handling system 20 to increase the rate of influx of air from outdoor environment 38 to reduce the concentration of the test gas in indoor environment 10. In embodiments, electronic controller 130 may activate a particle mitigation system, such as activating an exhaust fan to pull air out of indoor environment 10.

In embodiments, each of the settings of the particle mitigation systems in indoor environment 10 are static during the testing carried out herein for both test gases and test particles. Thus, air characterization system 100 can determine the effectiveness of the current settings and make recommendations based thereon.

Returning to FIG. 6, graph 520 includes a concentration over time of a test particle, represented by test particle curve 524, is shown. In embodiments, the presence of test particles from particle generator 102 allows electronic controller 130 to quantify the effectiveness of one or more of the particle mitigation systems, such as mechanical filters 30 of air handling system 20, air purifier 60, and light 80.

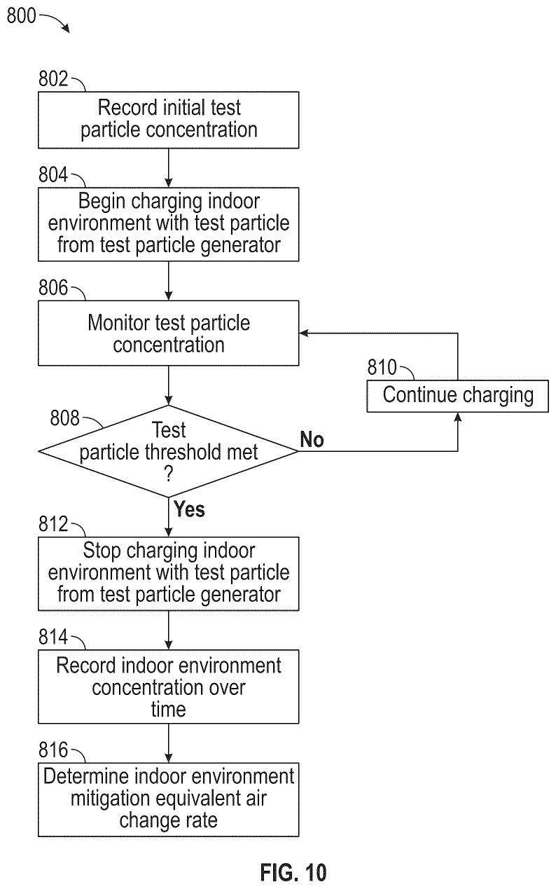

Referring to FIG. 10, an exemplary processing sequence 800 of electronic controller 130 is shown. An initial concentration of the test particles is recorded, as represented by block 802. In embodiments, a sensor 110 of particle monitor 104 records a signal representative of the concentration of the test particles just prior to charge region 532 in graph 520. Indoor environment is charged with test particles from particle generator 102, as represented by block 804. Electronic controller 130 records the amount of test particles released from particle generator 102 into indoor environment 10, as represented by block 806. If multiple charges are needed, electronic controller 130 loops back and requests that particle generator 102 release further charges of test particles to indoor environment 10, as represented by blocks 808 and 810. Once a threshold amount 532 (see FIG. 6) is achieved, in embodiments, electronic controller 130 stops charging indoor environment 10 with test particles from particle generator 102, as represented by block 812. Blocks 802, 804, 806, 808, 810, and 812 are collectively referred to as the charging portion of processing sequence 800 while the remaining blocks of processing sequence 800 are collectively referred to as the monitoring portion of processing sequence 800.

Electronic controller 130 uses particle monitor 104 to monitor the concentration of the test particles in indoor environment 10 over time, as represented by block 814. In embodiments, electronic controller 130 records the monitored concentration and time as ordered pairs of data 138 in memory 134.

Electronic controller 130 then determines the mitigation equivalent air change rate (MEACH) for air handling system 20 of indoor environment 10 and air purifier 60, if present, as represented by block 816. In embodiments, the MEACH also includes the mitigation by light 80 if present in indoor environment 10. In other embodiments, such as described in connection with FIG. 12 and processing sequence 860, the test particles have a characteristic that is responsive to the light emitted by light 80, such as a fluorescence dye, and particle monitor 104 is able to quantify the effectiveness of light 80 separate from air handling system 20 and air purifier 60.

In embodiments, the MEACH of air handling system 20 and air purifier 60 of indoor environment 10 may be determined by fitting the data points of curve 524 to equation 4:

$$C = ( \frac{D}{{Q}_{A}} + {C}_{B} ) ( 1 - {e}^{- MEACH * t} ) + {C}_{I} {e}^{- MEACH * t} \qquad ( 4 )$$

wherein C is the concentration of the test particles in indoor environment 10 (particles/m³), D is the release rate of the test particles in indoor environment 10 (particle/sec) of particle generator 102, Q_{A} is the air volume supply rate of particle generator 102 (m³/sec), C_{B} is the background concentration of the test particle in indoor environment 10 (particles/m³), MEACH is mitigation equivalent air change rate, t is the elapsed time (sec), and C, is the initial concentration of test particles in indoor environment 10 (particles/m³) at the beginning of post-charge region 534. Each of D and Q_{A}, are provided as inputs to air characterization system 100 through input devices 140. C_{B} may be measured by particle monitor 104 in pre-charge region 530 and recorded by air characterization system 100. CI is measured by particle monitor 104 and recorded by air characterization system 100. Each of C and t are ordered pairs of the data 138 stored in memory 134 that forms test particle curve 524. In embodiments, air characterization system 100 selects a value of MEACH by fitting the ordered pairs of data 138 to equation 4 and selecting the value of MEACH that reduces the error between the respective ordered pairs and equation 4. In embodiments, each ordered pair of C and t are used to compute a value of MEACH and an average of the MEACH values is reported by air characterization system 100 as the MEACH value of air handling system 20 and air purifier 60.

Referring to FIG. 11, another exemplary processing sequence 840 of electronic controller 130 is shown. Electronic controller 130 determines an indoor environmental air characteristic based on the tracking of a test gas and on the tracking of a test particle, as represented by block 842. In embodiments, one of processing sequence 700 and processing sequence 740 is used to determine the ACH of air handling system 20 and processing sequence 800 is used to determine the MEACH of the filters present in indoor environment 10, such as in air handling system 20 and air purifier 60. In embodiments, the MEACH may also be used to account for light 80 present in indoor environment 10.

Electronic controller 130 next determines if the determined air characteristic satisfies a criteria, as represented by block 844. In embodiments, the ACH and MEACH are summed and the result is compared to a threshold value. If the summed result is equal to or exceeds the threshold value then indoor environment 10 satisfies the criteria. If the summed result is less than the threshold value then indoor environment 10 does not satisfy the criteria.

If the criteria is satisfied, air characterization system 100 displays with output devices 142 the summed result and/or the components of the summed result, as represented by block 846 and continues to monitor once a subsequent testing period is initiated, as represented by block 848. If the criteria is not satisfied, air characterization system 100 may take additional steps to address the poor air characteristic, as represented by block 850. In embodiments, electronic controller 130 may output an alert with output devices 142, as represented by block 852. Exemplary alerts include activating a visual indicator, such as flashing a light or displaying a message on a screen, activating an audio indicator, such as emitting an audible tone or message, and/or sending a message to a remote device with an exemplary communication module.

Further, in embodiments, electronic controller 130 may modify one or mitigation settings for the particle mitigation systems of indoor environment 10, as represented by block 854. Further, electronic controller 130 may alter the mitigation settings in indoor environment 10 or provide guidance on potential changes to make to mitigation settings in the room. For example, electronic controller 130 may suggest adding additional air purifier 60 to indoor environment 10 or increasing the rate of influx of air from outdoor environment 38 by air handling system 20, both of which may reduce the concentration of the test particles in indoor environment 10. In examples, electronic controller 130 may send a signal to air handling system 20 to increase the rate of influx of air from outdoor environment 38 by air handling system 20 and/or electronic controller 130 may activate a particle mitigation system, such as activating an exhaust fan to pull air out of indoor environment 10.

Referring to FIG. 12, another exemplary processing sequence 860 of electronic controller 130 is shown which is generally the same as processing sequence 800. Blocks 802, 804, 806, 810, and 812 are collectively referred to as the charging portion of processing sequence 860 while the remaining blocks of processing sequence 860 are collectively referred to as the monitoring portion of processing sequence 860. In processing sequence 860, during the charging portion a test particle is used which is responsive to light 80, such as having a fluorescent dye that may be activated by light 80. Prior to beginning the monitoring portion of processing sequence 860 or at the beginning of the monitoring portion of processing sequence 860, light 80 is activated as represented by block 862. In embodiments, the particle monitor 104 used is a dual beam laser particle counter or a condensation particle counter in which the primary beam detects the test particles and a secondary beam causes fluorescence of the particles which have been altered by light 80 would allow for differentiation and measurement of fluorescent particle concentration and total particle concentration. Thus, two ordered pairs are recorded by particle monitor 104. A first ordered pair is the total particle concentration and time, represented by block 814. A second ordered pair is the fluorescing particle concentration and time, represented by block 864. The first ordered pairs may be used to determine the MEACH for indoor environment 10. The second ordered pairs may be used to determine a measure of the effectiveness of light 80 in altering the test particles.

Based on the effectiveness of light 80 in altering the test particles, electronic controller 130 may determine an effective inactivation of a second particle, such as an infectious particle by light 80, as represented by block 866, with the assumption that inactivation is functionally equivalent to removal such that a MEACH value of light 80 for the second particle may be determined. In embodiments, air characterization system 100 includes as part of data 138 a correlation table based on empirical data which defines the relationship between observed altering of first test particles by light 80 in a given setting and observed inactivation rates of a second particle, such as an infectious particle by light 80 in the same setting. If an observed alteration of the test particle is not specified in the correlation table, air characterization system 100 extrapolates the adjacent alteration values in the correlation table to determine an estimated MEACH value for the second particle. In one embodiment, air characterization system 100 performs a linear extrapolation.

Referring to FIG. 13, another exemplary processing sequence 880 of electronic controller 130 is shown. Electronic controller 130 determines an indoor environmental air characteristic based on the tracking of a test gas and on the tracking of a test particle, tracking of a test gas, and a particle inactivation characteristic, as represented by block 882. In embodiments, one of processing sequence 700 and processing sequence 740 is used to determine the ACH of air handling system 20 and processing sequence 800 is used to determine the MEACH of the filters present in indoor environment 10, such as in air handling system 20 and air purifier 60. Further, processing sequence 860 is used to determine the particle inactivation characteristic, such as an estimated MEACH value for a second particle based on the test particle.

Electronic controller 130 next determines if the determined air characteristic satisfies a criteria, such as for a second particle, as represented by block 884. In embodiments, air characterization system 100 determines if light 80 is active and is thus inactivating the second particle and the ACH and MEACH (for air handling system 20 and/or air purifier 60) are summed and the result is compared to a threshold value. If the summed result is equal to or exceeds the threshold value and light 80 is active then indoor environment 10 satisfies the criteria. If the summed result is less than the threshold value and/or light 80 is inactive then indoor environment 10 does not satisfy the criteria. In embodiments, the criteria is set based on whether ACH and MEACH (for air handling system 20 and/or air purifier 60) along with the estimated MEACH for the second particle by light 80 is sufficient for a target room occupancy for indoor environment 10.

If the criteria is satisfied, air characterization system 100 displays with output devices 142 the summed result and/or the components of the summed result, as represented by block 886 and continues to monitor once a subsequent testing period is initiated, as represented by block 888. If the criteria is not satisfied, air characterization system 100 may take additional steps to address the poor air characteristic, as represented by block 890. In embodiments, electronic controller 130 may output an alert with output devices 142, as represented by block 892. Exemplary alerts include activating a visual indicator, such as flashing a light or displaying a message on a screen, activating an audio indicator, such as emitting an audible tone or message, and/or sending a message to a remote device with an exemplary communication module.

Further, in embodiments, electronic controller 130 may modify one or mitigation settings for the particle mitigation systems of indoor environment 10, as represented by block 894. Further, electronic controller 130 may alter the mitigation settings in indoor environment 10 or provide guidance on potential changes to make to mitigation settings in the room. For example, electronic controller 130 may suggest adding an additional air purifier 60 to indoor environment 10 or increasing the rate of influx of air from outdoor environment 38 by air handling system 20, both of which may reduce the concentration of the test particles in indoor environment 10. In examples, electronic controller 130 may send a signal to air handling system 20 to increase the rate of influx of air from outdoor environment 38 by air handling system 20 and/or electronic controller 130 may activate a particle mitigation system, such as activating an exhaust fan to pull air out of indoor environment 10.

Referring to FIG. 14, another exemplary processing sequence 896 of electronic controller 130 is shown. Processing sequence 896 is generally the same as processing sequence 880 with the addition of a determination of an occupancy of indoor environment 10, as represented by block 898. As discussed herein, if the test gas used is carbon dioxide, air characterization system 100 able to determine an estimated occupancy level of indoor environment 10 based on a steady state value 538 compared to a steady state value for an unoccupied indoor environment 10. By monitoring the occupancy of indoor environment 10, air characterization system 100 may update the criteria for block 884 based on the estimated occupancy which can vary over time instead of using a target occupancy.

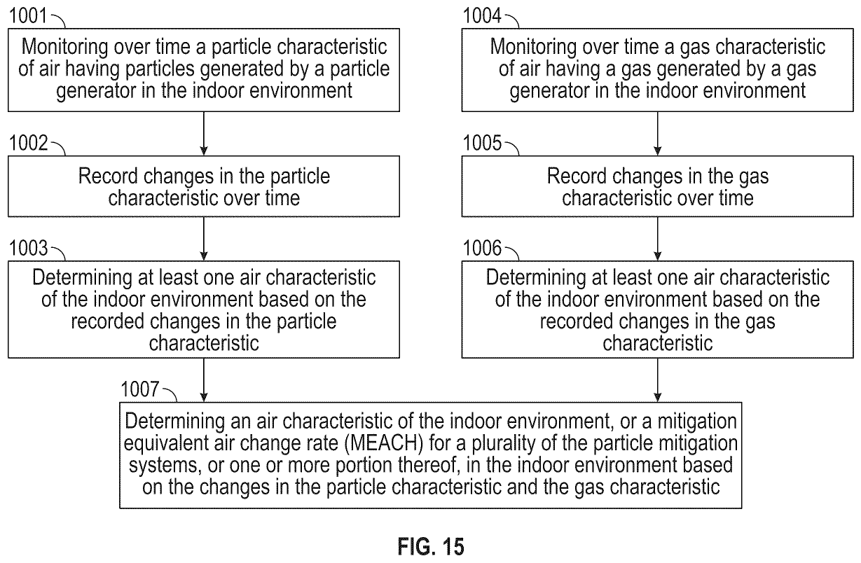

Referring to FIG. 15, an exemplary method of air characterization is shown, which utilizes both particle and gas measurements to determine an air characteristic of the indoor environment. A device, system, or user analyzes content of a given set of particles in air of the indoor environment. In step 1001, the method involves monitoring over time a particle characteristic of air having particles generated by a particle generator in the indoor environment. Examples of monitoring data over time include collection of time series data. In step 1002, the method involves recording changes in the particle characteristics over time. In step 1003, the method involves determining at least one air characteristic of the indoor environment based on the recorded changes in the particle characteristic. The device, system, or user also analyzes content of a given gas in the air of the indoor environment. In step 1004, the method involves monitoring over time a gas characteristic of air having a gas generated by a gas generator in the indoor environment. In step 1005, the method involves recording changes in the gas characteristic over time. In step 1006, the method involves determining at least one air characteristic of the indoor environment based on the recorded changes in the gas characteristic. The data obtained from analyzing the particles and gas are then used together to resolve and disambiguate the effect of mitigation efforts on clean air in the indoor space. In step 1007, the method involves determining an air characteristic of the indoor environment, or a mitigation equivalent air change rate (MEACH) for a plurality of the particle mitigation systems, or one or more portion thereof, in the indoor environment based on the changes in the particle characteristic and the gas characteristic.

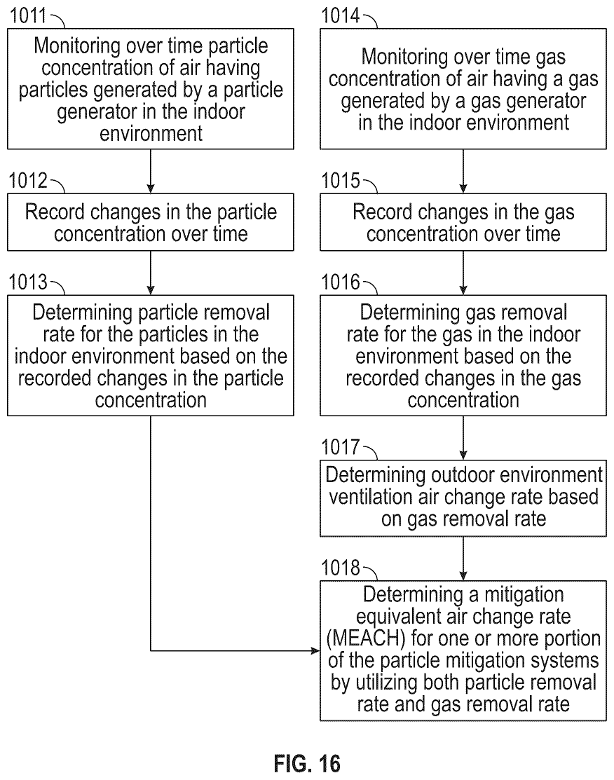

Referring to FIG. 16, an exemplary method of air characterization is shown, which utilizes an air change rate based on gas removal to determine a mitigation equivalent air change rate (MEACH) for one or more portion of a plurality of particle mitigation systems. A device, system, or user analyzes content of a given set of particles in air of the indoor environment. In step 1011, the method involves monitoring over time particle concentration of air having particles generated by a particle generator in the indoor environment. In step 1012, the method involves recording changes in the particle concentration over time. In step 1013, the method involves determining particle removal rate for the particles in the indoor environment based on the recorded changes in the particle concentration. The device, system, or user also analyzes content of a given gas in the air of the indoor environment. In step 1014, the method involves monitoring over time gas concentration of air having a gas generated by a gas generator in the indoor environment. In step 1015, the method involves recording changes in the gas concentration over time. In step 1016, the method involves determining gas removal rate for the gas in the indoor environment based on the recorded changes in the gas concentration. In step 1017, the method involves determining outdoor environment ventilation air change rate based on gas removal rate. The data obtained from analyzing the particles and gas are then used together to resolve and disambiguate the effect of mitigation efforts on clean air in the indoor space. In step 1018, the method involves determining a mitigation equivalent air change rate (MEACH) for one or more portion of the particle mitigation systems by utilizing both particle removal rate and gas removal rate.

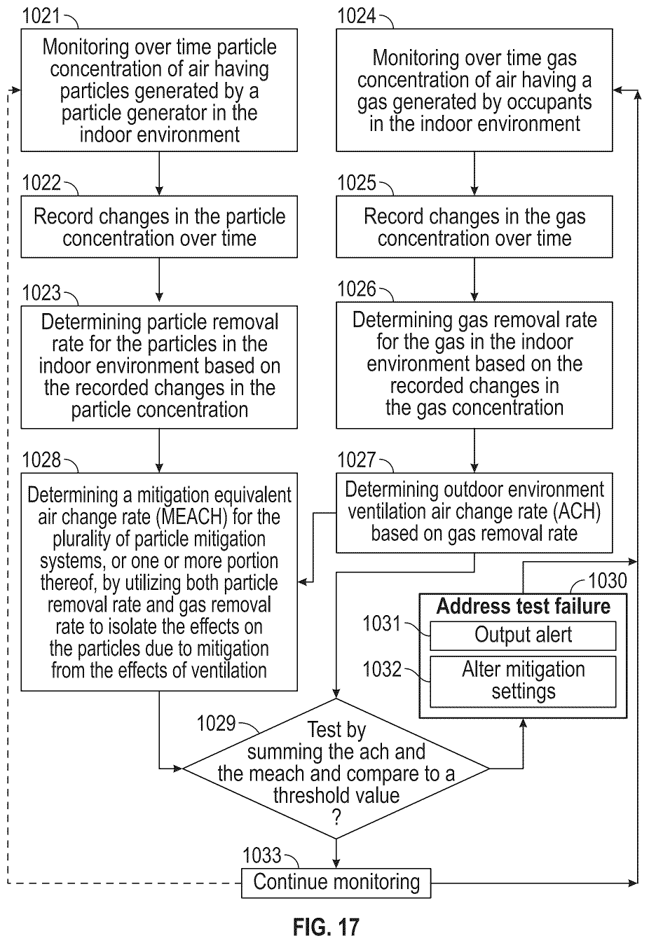

Referring to FIG. 17, an exemplary method of air characterization is shown, which utilizes an air change rate based on gas removal of a gas generated by occupants together with a MEACH for a plurality of particle mitigation systems, or one or more portion thereof, for repeatable testing and system adjustment in an indoor environment. A device, system, or user analyzes content of a given set of particles in air of the indoor environment. In step 1021, the method involves monitoring over time particle concentration of air having particles generated by a particle generator in the indoor environment. In step 1022, the method involves recording changes in the particle concentration over time. In step 1023, the method involves determining particle removal rate for the particles in the indoor environment based on the recorded changes in the particle concentration. The device, system, or user also analyzes content of a gas in the air of the indoor environment generated by occupants in the indoor environment. In step 1024, the method involves monitoring over time gas concentration of air having a gas generated by occupants in the indoor environment. In step 1025, the method involves recording changes in the gas concentration over time. In step 1026, the method involves determining gas removal rate for the gas in the indoor environment based on the recorded changes in the gas concentration. In step 1027, the method involves determining outdoor environment ventilation air change rate (ACH) based on gas removal rate. The data obtained from analyzing the particles and gas are then used together to resolve and disambiguate the effect of mitigation efforts on clean air in the indoor space. In step 1028, the method involves determining a mitigation equivalent air change rate (MEACH) for the plurality of particle mitigation systems, or one or more portion thereof, by utilizing both particle removal rate and gas removal rate to isolate the effects on the particles due to mitigation from the effects of ventilation. The MEACH data and the ventilation (ACH) data can be used together to evaluate air quality. In step 1029, the method involves a testing step that can be performed by summing the ACH and the MEACH and comparing the resulting sum to a threshold value. In step 1030, if the resulting sum fails to satisfy the threshold value, the device, user, or system can issue an alert 1031 or alter one or more mitigation settings 1032 associated with particle removal in the indoor environment, such as filter settings or air handling settings. In step 1031, if the resulting sum is satisfactory, the system may proceed to continue monitoring 1033. Such continuous monitoring can proceed in continuous manner identifying when air quality is at, above, or below satisfactory performance—with or without continuous further measuring of particles. In various examples, particle monitoring can be time coordinated with gas monitoring, such as simultaneous or non-simultaneous. In various examples, each particle monitoring can be simultaneous or have at least partially overlapping time frames with a gas monitoring step. In various examples, gas monitoring can occur more often than particle monitoring. In various examples, gas monitoring occurs for each instance of particle monitoring or recorded particle data. In various examples, particle monitoring does not occur for each instance of gas monitoring or recorded gas data. This configuration advantageously can provide a flexible, streamlined and repeatable process. System for determining when an indoor space is achieving, or failing to achieve, a target air quality all by use of gas monitoring.

In step 1032, if the resulting sum is satisfactory, the system may proceed to continue monitoring 1033. Such continuous monitoring can proceed in continuous manner identifying when air quality is at, above, or below satisfactory performance-even without further measuring of particles. This configuration advantageously can provide a streamlined and repeatable process. system for determining when an indoor space is achieving, or failing to achieve, a target air quality all by use of gas monitoring

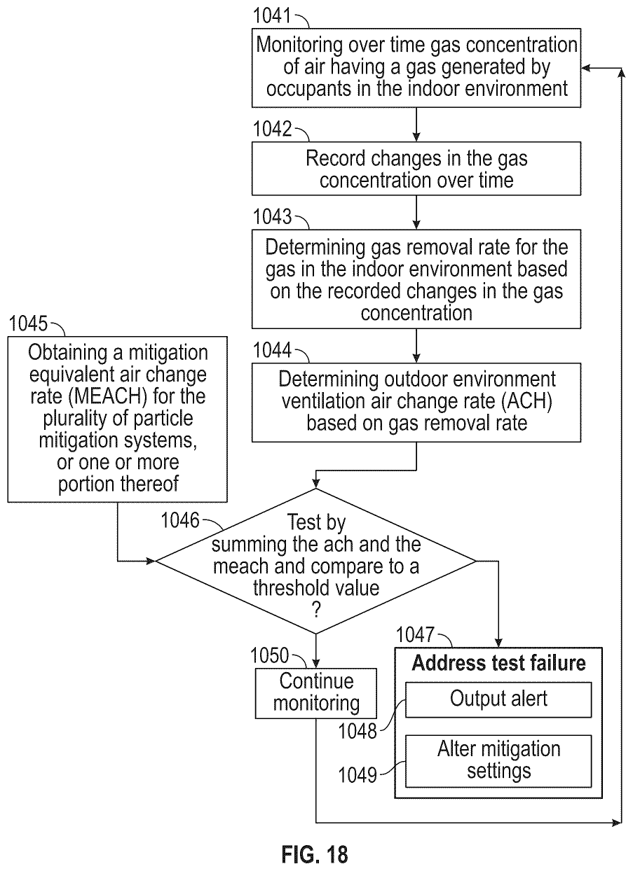

Referring to FIG. 18, the device, system, or user analyzes content of a gas in the air of the indoor environment generated by occupants in the indoor environment. The device, system, or user analyzes content of a gas in the air of the indoor environment generated by occupants in the indoor environment. In step 1041, the method involves monitoring over time gas concentration of air having a gas generated by occupants in the indoor environment. In step 1042, the method involves recording changes in the gas concentration over time. In step 1043, the method involves determining gas removal rate for the gas in the indoor environment based on the recorded changes in the gas concentration. In step 1044, the method involves determining outdoor environment ventilation air change rate (ACH) based on gas removal rate. In step 1045, a device, system, or user provides a mitigation equivalent air change rate (MEACH), for example as a user or system input value, for the plurality of particle mitigation systems, or one or more portion thereof. The MEACH data and the ventilation (ACH) data can be used together to evaluate air quality. In step 1046, the method involves a testing step that can be performed by summing the ACH and the MEACH and comparing the resulting sum to a threshold value. The combination of the ACH corresponding to ventilation and the MEACH corresponding to mitigation-based particle removal can provide an assessment of the air quality of the indoor environment overall. For example, combining the ACH and MEACH can the clean air delivery rate (CADR). In various aspects, it can be more advantageous to utilize the combination of ACH derived from gas monitoring with the MEACH of the particle mitigation systems than it is to simply use a MEACH based on particle removal overall determined simply by measuring particles. Although measuring particles only can give a result for CADR (capturing all particle removal whether by ventilation or mitigation); surprisingly, it can be more effective to instead determine CADR from a combination of MEACH based on both particle and gas and then adding in the ACH representing the effect of ventilation. The basis for this advantage relates to the time frames of variation and accuracy of particle and gas measurements. Due to high gas concentrations and the speed and accuracy of gas-based processes, it can be advantageous to utilize changes in gas characteristics as a determinative step for air quality monitoring-even though particles are the component of interest. When comparing air quality against threshold values in can also be advantageous to have separate constituent values corresponding to ventilation and particle mitigation systems in order to determine which adjustments are necessary to improve air quality. In step 1047, if the resulting sum fails to satisfy the threshold value, the device, user, or system can issue an alert 1048 or alter one or more mitigation settings 1049 associated with particle removal in the indoor environment, such as filter settings or air handling settings. When separate values are known for ventilation and particle mitigation systems, the appropriate step for improving air quality can be determined. For example, ventilation can be increased or decreased, fan speeds for an air handler or a filtration system can be increased or decreased, and light systems can be adjusted. In step 1048, an alert is transmitted to a device, user, or system to notify that one or more air quality criteria are not met. In step 1049, instructions are transmitted to a device, user, or system to change one or more setting of the plurality of particle mitigation systems, or portion thereof, or to adjust ventilation for the indoor space. In step 1050, after satisfying the one or more criteria for air characteristics or after upon addressing a failure, the system continues with further monitoring. The process can repeat to provide a continuous process. Such continuous monitoring can proceed in continuous manner identifying when air quality is at, above, or below satisfactory performance-even without further measuring of particles. This configuration advantageously provides an efficient repeatable process. Moreover, the process can permit continuous monitoring air quality and identification of appropriate adjustments to the particle mitigation systems, without requiring particle monitoring every cycle. This improved efficient can improve real-time or near real-time monitoring. In various aspects, ventilation and its contribution to ACH can be highly variable on short time frames, while non-ventilation MEACH contribution of particle mitigation systems generally varies on a longer time scale. It can be advantageous to monitor ventilation more often than the non-ventilation contribution of particle mitigation systems. Similarly, it can be advantageous to monitor gas on a shorter time scale than particles are monitoring. It can be advantageous to monitor gas continuously and monitor particles less than continuously. In various aspects, the ACH can be determined from gas monitoring by measuring the gas characteristic about, at least, or less than, 0.001, 0.01, 0.1, 0.3, 0.4, 0.5, 0.6, 0.7, 0.8, 0.9, 1, 2, 3, 4, 5, 6, 7, 8, 9, 10, 20, 30, 40, 50, 60, 120, 180, 240, 300, 360, 420, 480, 540, 600, 900, or 1200 seconds before assessing air quality or comparison to a criteria value. for the quality all by use of gas monitoring. For example, the MEACH determined from measuring the particle characteristic, at least, or less than, 0.1, 0.3, 0.4, 0.5, 0.6, 0.7, 0.8, 0.9, 1, 2, 3, 4, 5, 6, 7, 8, 9, 10, 20, 30, 40, 50, 60, 120, 180, 240, 300, 360, 420, 480, 540, 600, 900, 1200, or 1440 minutes before assessing air quality or comparison to a criteria value. In further examples, the gas characteristic is measured for each instance of determining ACH, MEACH, air quality, a criteria, or a combination thereof. In further examples, the particle characteristic is measured less often than the gas characteristic, or less than often than once for each instance of determining ACH, MEACH, air quality, a criteria, or a combination thereof.

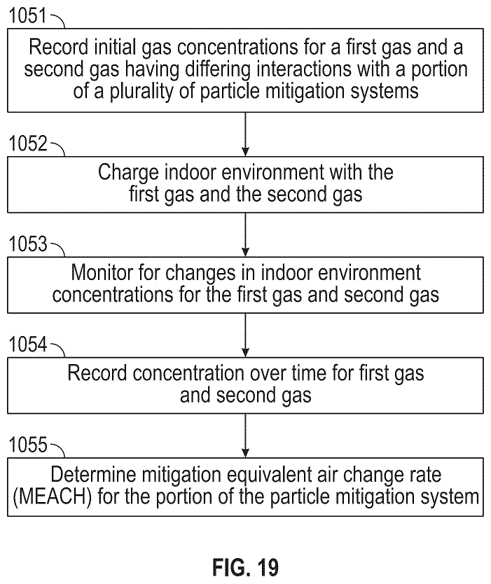

Referring to FIG. 19, an exemplary method of air characterization is shown, which utilizes a first gas and a second gas having differing interactions with a portion of a plurality of particle mitigation systems to determine a mitigation equivalent air change rate (MEACH) for said portion of the particle mitigation system. The method analyzes content of a first gas and a second gas in the air of the indoor environment generated by occupants in the indoor environment. One of the advantages of using two gases is a user, device, system, or process can disambiguate the performance of the various posters by using two gases having differing interactions with a portion of a plurality of particle mitigation systems. For example, in step 1051, the method involves recording initial gas concentrations for a first gas and a second gas having differing interactions with a portion of a plurality of particle mitigation systems. For example, a portion of the particle mitigation systems can be a scrubber, activated carbon, or other type of component configured to remove a gas from air of the indoor environment. As such, that removed gas will reflect different particle removal rate than another gas that is not removed by the same component. In step 1052, the method involves charging indoor environment with the first gas and the second gas. This charging process can be supplied from gas sources: such as pressurized gas cannisters, or it can be supplied from human occupants, which are monitored in the indoor environment. In step 1, the method involves monitor for changes in indoor environment concentrations for the first gas and second gas. In step 1054, the method involves recording concentration over time for first gas and second gas. In step 1055, the method involves determine mitigation equivalent air change rate (MEACH) for the portion of the particle mitigation system. Specifically, the difference in removal rate between the first gas and the second gas reflects the contribution of the gas-removing portion of the particle mitigation system. Here, it is suitable to use MEACH to describe the mitigation equivalent effect with respect to the removable gas particle and the referenced particle mitigation system or portion thereof.

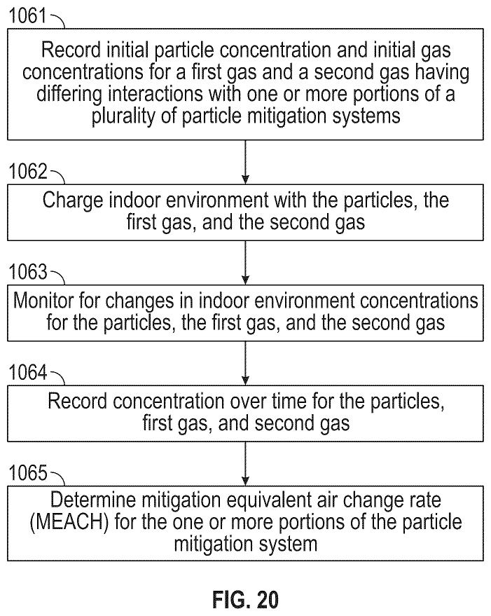

Referring to FIG. 20, an exemplary method of air characterization is shown, which utilizes particles, a first gas, and a second gas having differing interactions with one or more portion of a plurality of particle mitigation systems to determine a mitigation equivalent air change rate (MEACH) for the one or more portion of the particle mitigation system. In step 1061, the method involves recording initial particle concentration and initial gas concentrations for a first gas and a second gas having differing interactions with one or more portions of a plurality of particle mitigation systems. In step 1062, the method involves charging indoor environment with the particles, the first gas, and the second gas. In step 1063, the method involves monitoring for changes in indoor environment concentrations for the particles, the first gas, and the second gas. In step 1064, the method involves recording concentration over time for the particles, first gas, and second gas. In step 1065, the method involves determining mitigation equivalent air change rate (MEACH) for the one or more portions of the particle mitigation system.

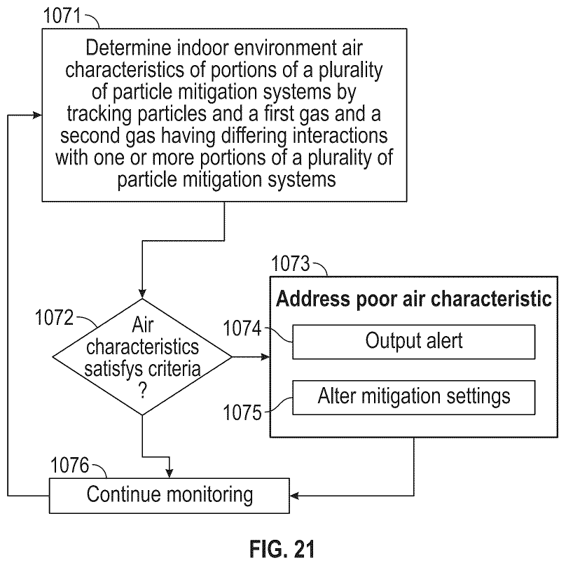

Referring to FIG. 21, an exemplary method of air characterization is shown, which utilizes particles, a first gas, and a second gas having differing interactions with a portion of a plurality of particle mitigation systems to assess an air quality criteria for repeatable testing and system adjustment in an indoor environment. In step 1071, the method involves determine indoor environment air characteristics of portions of a plurality of particle mitigation systems by tracking particles and a first gas and a second gas having differing interactions with one or more portions of a plurality of particle mitigation systems. In step 1072, the method involves checking to see whether one or more air characteristics satisfy criteria. In step 1073, the method involves addressing any air characteristics that do not satisfy the criteria, such as by transmitting an alert or by adjusting one or more setting of the plurality of air mitigation systems, or a portion thereof. In step 1074, upon satisfying the one or more criteria for air characteristics, transmitting an alert, or altering one or more air mitigation setting, the system continues with further monitoring. The process can repeat to provide a continuous process.

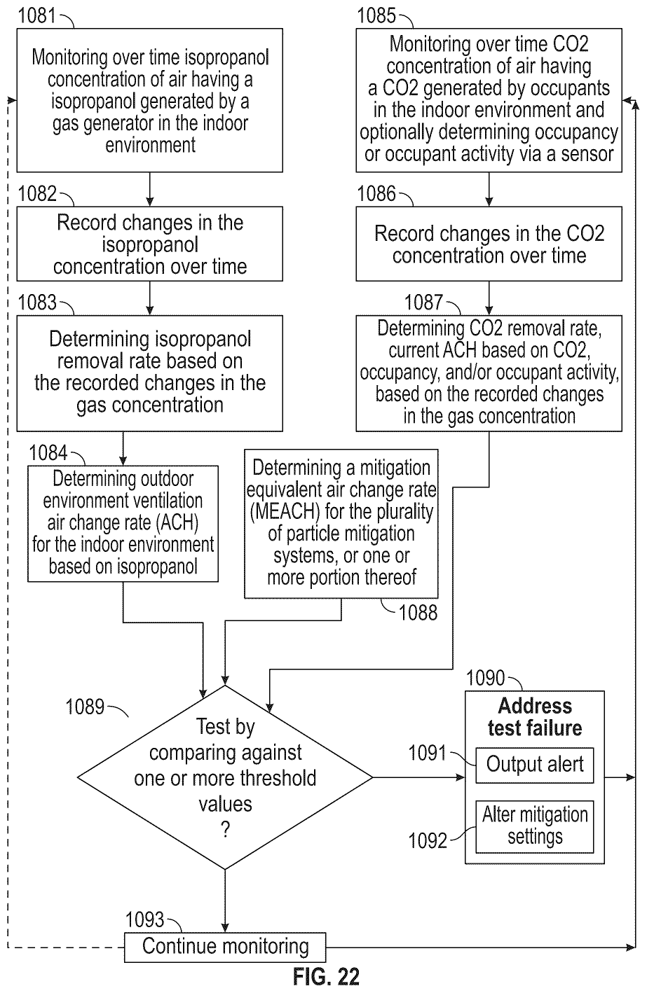

Referring to FIG. 22, an exemplary method of air characterization is shown, which utilizes a first gas (isopropanol) and a second gas (CO2) generated by occupants, together with a MEACH for a plurality of particle mitigation systems, or one or more portion thereof, for repeatable testing and system adjustment in an indoor environment via CO2 monitoring. In step 1081, the method involves monitoring over time isopropanol concentration of air having a isopropanol generated by a gas generator in the indoor environment. In step 1082, the method involves recording changes in the isopropanol concentration over time. In step 1083, the method involves determining isopropanol removal rate based on the recorded changes in the gas concentration. In step 1084, the method involves determining outdoor environment ventilation air change rate (ACH) for the indoor environment based on isopropanol. In step 1085, the method involves monitoring over time CO2 concentration of air having a CO2 generated by occupants in the indoor environment and optionally determining occupancy or occupant activity via a sensor. In step 1086, the method involves recording changes in the CO2 concentration over time. In step 1087, the method involves determining CO2 removal rate, current ACH based on CO2, occupancy, and/or occupant activity, based on the recorded changes in the gas concentration. In step 1088, the method involves determining a mitigation equivalent air change rate (MEACH) for the plurality of particle mitigation systems, or one or more portion thereof. In step 1089, the method involves testing one or more determined characteristic against a criteria or threshold value. For example, air quality of air in the indoor environment can assessed by summing the ACH and the MEACH and comparing to a threshold value while adjusting for current CO2 removal rate, occupancy, and/or occupant activity. As another example, performance of a gas-removing component of the particle mitigation system can be assessed by taking the difference between the first gas removal rate and second removal rate. In step 1090, the method involves addressing the test failure by transmitting an output alert (as in step 1091) and/or by altering one or more setting (as in 1092) of the plurality of air mitigation systems, or a portion thereof. For example, the method can involve transmitting instructions to increase or decrease fan speed in a mechanical filtration system or air handling system, increase or decrease ventilation, increase or decrease UV light provided via a light system, change filters, add filters, and the like. In step 1093, upon satisfying the one or more test criteria or addressing a failed test, the system continues with further monitoring. The process can repeat to provide a continuous process. Such continuous monitoring can proceed in continuous manner identifying when air quality is at, above, or below satisfactory performance-even with fewer, less frequent, or no additional measurements of particles and isopropanol. In various examples, particle monitoring can be time coordinated with gas monitoring, such as simultaneous or non-simultaneous. In various examples, each particle monitoring can be simultaneous or have at least partially overlapping time frames with a gas monitoring step. In various examples, gas monitoring can occur more often than particle monitoring. In various examples, gas monitoring occurs for each instance of particle monitoring or recorded particle data. In various examples, particle monitoring does not occur for each instance of gas monitoring or recorded gas data. As one example, a gas monitor detecting isopropanol can be used to identify room volume, a particle monitor can be used to identify MEACH of the particle mitigation systems together with as gas monitor, and then CO2 can be continuously monitored to detect changes in ventilation reflection changes in air quality or other air characteristic of the indoor environment. This configuration advantageously provides an efficient repeatable process that can permit continuous monitoring air quality and identification of appropriate adjustments to the particle mitigation systems, without requiring particle monitoring every cycle. In various aspects, this improved efficiency can improve real-time or near real-time monitoring. Ventilation particle monitoring or updated MEACH values are on a longer time frame than gas monitoring or updated gas values.

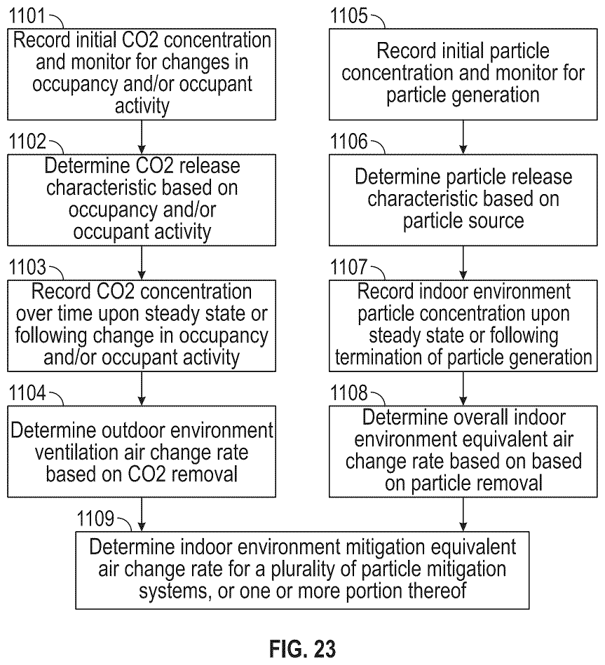

Referring to FIG. 23, an exemplary method of air characterization is shown, which utilizes occupant monitoring, CO2 monitoring, and particle monitoring, to determine a mitigation equivalent air change rate (MEACH) for a plurality of particle mitigation systems, or one or more portions thereof. In step 1101, the method involves recording initial CO2 concentration and monitor for changes in occupancy and/or occupant activity. In step 1102, the method involves determining CO2 release characteristic based on occupancy and/or occupant activity. In step 1103, the method involves recording CO2 concentration over time upon steady state or following change in occupancy and/or occupant activity. In step 1104, the method involves determining outdoor environment ventilation air change rate based on CO2 removal. In step 1105, the method involves recording initial particle concentration and monitor for particle generation. In step 1106, the method involves determining particle release characteristic based on particle source. In step 1107, the method involves recording indoor environment particle concentration upon steady state or following termination of particle generation. In step 1108, the method involves determining overall indoor environment equivalent air change rate based on based on particle removal. In step 1109, the method involves determining indoor environment mitigation equivalent air change rate for a plurality of particle mitigation systems, or one or more portion thereof.

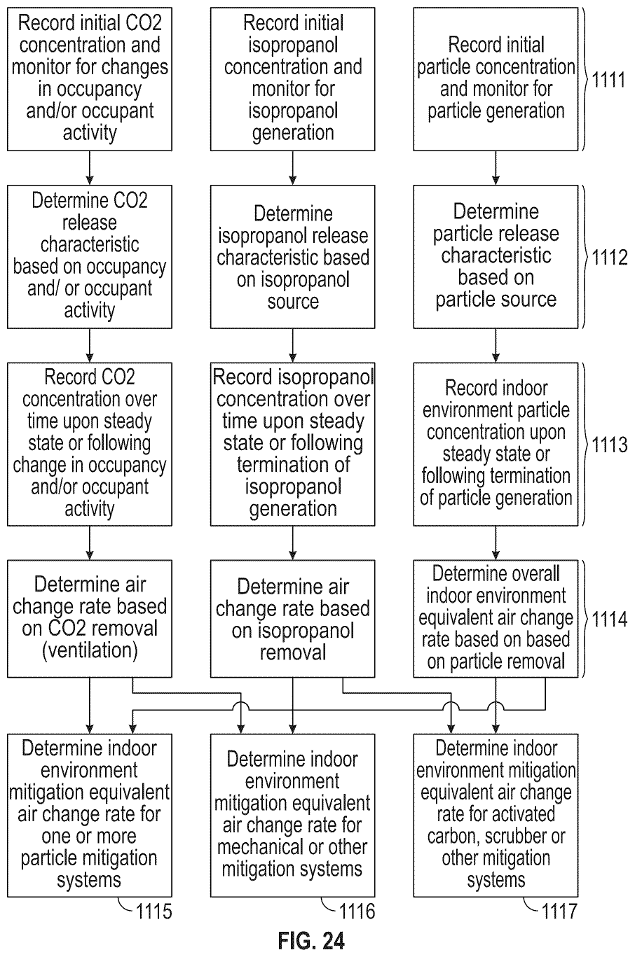

Referring to FIG. 24, an exemplary method of air characterization is shown, which utilizes occupant monitoring, a first gas (CO2) monitoring, a second gas (isopropanol) monitoring, and particle monitoring, to determine a mitigation equivalent air change rate (MEACH) for portions of a plurality of particle mitigation systems such as mechanical filtration, scrubber, or electrostatic systems. In steps 1111, the method involves recording initial CO2 concentration and monitor for changes in occupancy and/or occupant activity, recording initial isopropanol concentration and monitor for isopropanol generation, and recording initial particle concentration and monitor for particle generation. In steps 1112, the method involves determining CO2 release characteristic based on occupancy and/or occupant activity, determining isopropanol release characteristic based on isopropanol source, and determining particle release characteristic based on particle source. In steps 1113, the method involves recording CO2 concentration over time upon steady state or following change in occupancy and/or occupant activity, recording isopropanol concentration over time upon steady state or following termination of isopropanol generation, and recording indoor environment particle concentration upon steady state or following termination of particle generation. In steps 1114, the method involves determining air change rate based on isopropanol removal, determining air change rate based on CO2 removal (ventilation), and determining overall indoor environment equivalent air change rate based on based on particle removal. In steps 1115, the method involves determining indoor environment mitigation equivalent air change rate for one or more particle mitigation systems based on the overall indoor environment equivalent air change rate based on based on particle removal together with the air change rate based on isopropanol removal. In steps 1116, the method involves determining indoor environment mitigation equivalent air change rate for a scrubber system (e.g., scrubbers configured for removing VOCs, activated carbon, or an ozone removing system) based on the air change rate based on isopropanol removal together with the air change rate based on CO2 removal, or alternatively based on the difference between the MEACH determined from isopropanol and the MEACH determined from CO2. In steps 1117, the method involves determining indoor environment mitigation equivalent air change rate for mechanical filtration or other mitigation systems based on the overall indoor environment equivalent air change rate based on based on particle removal together with the air change rate based on CO2 removal.

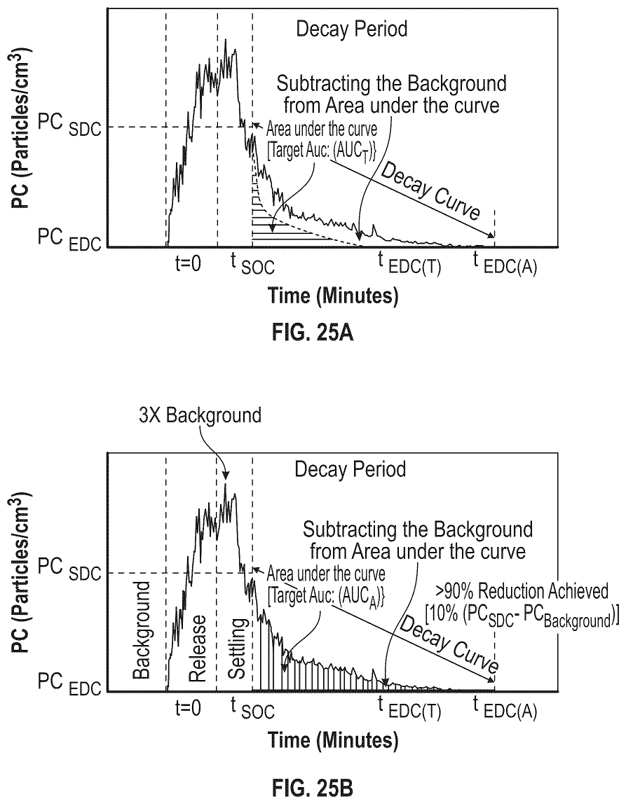

Referring to FIG. 25A and FIG. 25B, example charts are provided that illustrate example approaches to determine removal rate based on recording gas or particle characteristics in air of an indoor space.

Removal rate terms based on gas characteristcs (e.g., gas removal rate, ventilation rate, and ACH based on gas concentration) can be determined by obtaining a concentration curve based on gas data captured over time (i.e., time series data) by one or more gas monitors. Characteristics can be extracted from the resulting concentration curve such as area-under-the-curve (or “AUC”), maximum concentration, minimum concentration, initial concentration, final concentration, deviation from stead state concentration, amount above baseline concentration, multiple above baseline concentration, a baseline concentration, a decay rate (e.g., a slope of the curve), ACH, ventilation, a volumetric airflow rate for the indoor environment, or any combination thereof. For example, the area-under-the-curve of the time series data recorded during gas decay decay, as illustrated in FIG. 25B, can be used for determining amount and rate of gas removal or determining air quality such a volumetric air flow or equivalent. Another way to determine a removal rate based on gas characteristics characteristics is by obtaining a concentration curve based on time series gas data captured by one or more gas monitors and fitting an exponential to the decay curve to determine a removal rate. In various aspects, where a volume of the room space is utilized to determine such values, the room volume can be determined by a method described herein via use of a known bolus rather than physical measurement of the space. Passive monitoring and determination of removal rate is possible by using computer analysis to identify when gas concentration has entered a decay period, reached a threshold value (e.g., 3× above baseline), or both. In various aspects, gas removal rate is determined in a manner that is time-coordinated with the particle removal rate, for example the time window for calculating particle removal rate (i) is partially or fully overlapping with the time window for calculating particle removal rate, (ii) begins at the same time as the beginning of the time window for calculating particle removal rate, or (iii) is within a threshold period of time before or after the as the beginning of the time window for calculating particle removal rate.

Removal rate terms based on particle characteristcs (e.g., particle removal rate, overall particle removal rate, CADR, MEACH values such as MEACHᵣₒₒₘ, MEACH_{system}, MEACH_{filters}, etc., based on particle concentration) can be determined by obtaining a concentration curve based on particle data captured over time (i.e., time series data) by one or more particle monitors. Characteristics can be extracted from the resulting concentration curve such as area-under-the-curve (or “AUC”), maximum concentration, minimum concentration, initial concentration, final concentration, deviation from steady state concentration, amount above baseline concentration, multiple above baseline concentration, a baseline concentration, a decay rate (e.g., a slope of the curve), ACH, ventilation, a volumetric airflow rate for the indoor environment, or any combination thereof. For example, the area-under-the-curve of the time series data recorded during particle decay decay, as illustrated in FIG. 25B, can be used for determining amount and rate of particle removal or determining air quality such a volumetric air flow or equivalent. Another way to determine a removal rate based on particle characteristics characteristics is by obtaining a concentration curve based on time series particle data captured by one or more particle monitors and fitting an exponential to the decay curve to determine a removal rate. In various aspects, where a volume of the room space is utilized to determine such values, the room volume can be determined by a method described herein via use of a known bolus rather than physical measurement of the space. Passive monitoring and determination of removal rate is possible by using computer analysis to identify when particle concentration has entered a decay period, reached a threshold value (e.g., 3× above baseline), or both. In various aspects, particle removal rate is determined in a manner that is time-coordinated with the gas removal rate, for example the time window for calculating particle removal rate (i) is partially or fully overlapping with the time window for calculating gas removal rate, (ii) begins at the same time as the beginning of the time window for calculating gas removal rate, or (iii) is within a threshold period of time before or after the as the beginning of the time window for calculating gas removal rate.

Yet further ways to determine an air characteristic based on measuring gas in air is by use of techniques described in ASTM E741 or ASHRAE Standard 241, portions thereof, or adaptations thereof. Another way One way to determine an air characteristic based on measuring particles in air is by use of techniques described in ASHRAE Standard 241, portions thereof, or adaptations thereof. ASTM E741 and ASHRAE Standard 241 is incorporated by reference herewith, and the techniques described therein may be used in whole or in part for calculating, measuring, and determining one or more air characteristic, particle characteristic, or gas characteristic.

ASHRAE Standard 241 describes a highly formal standard for determining efficacy of particle mitigation efforts. However, as described herein, coupling gas monitoring together with particle monitoring solves several of the problems encountered when seeking to evaluate indoor environments that experience can use, activity, occupancy changes, ventilation changes, or environment fluctuations. Those of ordinary skill in the art will readily understand that use of gas monitoring to determine ACH and account for ventilation can streamline the type of calculations, determinations, and techniques utilized in ASHRAE Standard 241. For example, techniques in ASHRAE Standard 241 generally suffer from having restrictions on ventilation, air handling, room use, occupancy, and generally any sort of environmental fluctuation during test conditions. The consequences of these problems are at least two-fold: (1) such limitations can restrict the utility, circumstances, and environment where testing can occur, and (2) the approach fails to account for the contribution of ventilation that is present in many indoor environments. In many real-life situations, a more efficient mitigation approach can be achieved by determining both current ventilation and current mitigation performance, and the cost effects of over-mitigation and over ventilation can be avoided.

For reference, ASHRAE Standard 241 describes a technique for determining air cleaning system effectiveness and safety as follows. Currently, the consensus standards that can be used for determining effectiveness are: ANSI/ASHRAE Standard 52.2, ISO 16890-1, ANSI/AHAM AC-1, ANSI/AHAM AC-5, and ANSI/AHRAE 185.1. Currently, the consensus standards that can be used for determining safety are: UL 2998, ASTM D8407, and ISO 14644. Testing for effectiveness and safety can be performed using identical operating conditions of the air cleaning system equipment, but the testing is typically performed separately. The environmental and airflow conditions in the test environment can be equivalent and correspond to the intended application setting. Performance tests of air cleaning systems, whether mounted inside an air-handling unit (AHU) ductwork or plenum or placed in the occupied zone, can be performed as follows and the test chamber can comply with the following:
a. Volume can be at least 800 ft3 (22.7 m3). For air cleaning systems with an operational volume greater than 800 ft3 (22.7 m3), the ratio of the test chamber volume to the air cleaning system equivalent clean airflow rate can be greater than 1.7 for I-P units (ft3/cfm) or 0.101 for SI units (m3/[L/s]).
b. Surfaces can be smooth, nonporous, produce minimal emissions, and react minimally with the air cleaning system.
c. Surfaces can be electrically grounded.
d. Shall have fans to provide sufficient mixing, as defined in ASTM Standard D6670 26, Section 8.4, or equivalent.
e. Shall be sufficiently airtight during testing (leakage of less than 0.05 ach).
f. Shall be capable of flushing or treating air between tests.
g. Shall maintain temperature throughout the duration of testing at 73° F.±5° F. (23° C.±3° C.).
h. Shall maintain relative humidity throughout the duration of testing at 50%±10%.
i. Air-handling systems associated with chamber ventilation can be powered off during testing to minimize recirculation, unless testing with a side duct is necessary in which case air-handling systems intended to clean the air before or after tests can be powered off during the testing.

For in-duct air cleaning systems that clean air in the AHU, ductwork, or plenum, that can be tested for a single-pass infectious aerosol removal efficiency, the test duct can be as described in ANSI/ASHRAE Standard 52.2 5 ANSI/ASHRAE Standard 185.1 10, or per the system manufacturer's published specifications. For in-duct air cleaning systems that clean air in the AHU, ductwork, or plenum, that can be tested for a single-pass infectious aerosol removal efficiency, the test duct can be as described in ANSI/ASHRAE Standard 52.2 5 ANSI/ASHRAE Standard 185.1 10, or per the system manufacturer's published specifications. Air cleaning system equipment can be installed in the test chamber or test duct following the manufacturer's published specifications and in a manner that minimizes impacts on other testing procedures. The third-party independent test laboratory can certify that the installation meets these conditions or describe any potential impacts in the testing report. To ensure the quality of testing, the following measures can be taken:
a. All equipment used in testing can be calibrated per manufacturer instructions.
b. All equipment and surfaces can be cleaned and sterilized between tests as necessary to eliminate cross contamination between tests.
c. The test equipment can be placed within the chamber prior to testing.
d. Samples can be collected, handled, and analyzed in the same manner for all test iterations.
e. Background samples for each sampling target during testing (microbiological or chemical analyte) can be collected prior to each test.
f. A minimum of three replicates for each test condition (air cleaning system OFF and air cleaning system ON) can be performed, for a minimum of six total tests per air cleaning system. Effectiveness can be calculated using an average of these replicates.

Effectiveness testing of particle mitigation systems can be performed following the methodology of a standard described herein with a challenge microorganism, or conform to the following:
(1) All bioaerosol testing and microbial procedures can be conducted in accordance with the current version of the CDC/NIH Biosafety in Microbiological and Biomedical Laboratories
(2) All effectiveness testing utilizing microorganisms can be performed with the nonenveloped bacteriophage MS2 (host Escherichia coli). The test microorganism can be aerosolized by nebulizing a microbial suspension to produce discrete particles. Bioaerosol samples can be collected between 48 and 50 in. (123 and 127 cm) above the floor and >2 ft (0.6 m) from walls or equipment using impingers, impactors, or other acceptable sampling methods as determined by the third-party testing laboratory. The test microorganism samples can be collected, handled, and analyzed in the same manner for all test iterations. Each collected sample can be plated in triplicate.
(3) The effectiveness calculations can:
a. Account for background reduction of the test microorganism
b. Compare averages of the replicates with the air cleaning system OFF to the averages of the replicates with the air cleaning system ON
c. For microorganism recoveries lower than the limit of detection, use the value of the limit of detection for effectiveness calculations

The effectiveness of an air cleaning system operating within a room can be reported as an equivalent clean airflow delivery rate, determined using the procedure presented in Stephens, B., E. T. Gall, M. Heidarinejad, and D. K. Farmer. 2022. Interpreting mitigation systems performance data. ASHRAE Journal 64 (3): 20-30, incorporated by reference herewith and the following Equation 5:

$${V}_{ACS} - V ( {k}_{rd} - {k}_{nd} ) \qquad ( 5 )$$

where:
VACS=air cleaning system equivalent clean airflow rate, cfm (L/s)
V=test chamber volume, ft3 (L) ktd knd=infectious microorganism decay rate with air cleaning system operating, minute-1 (s-1)
k_{td}=infectious microorganism decay rate with air cleaning system operating, minute-1 (s-1)
k_{nd}=infectious microorganism decay rate without air cleaning system operating, minute-1 (s-1)

The effectiveness of an air cleaning system located in an AHU, ductwork, or plenum can be determined in accordance with:
a. Single-pass tests, such as those described in ANSI/ASHRAE Standard 52.2 5, ANSI/ASHRAE Standard 185.1 10, or equivalent, and reported as an infectious aerosol reduction efficiency (EPR). This can apply to in-duct air cleaning systems that clean the air in the AHU, ductwork, or plenum.
b. Tests meeting the conditions in Normative Appendix A with test air recirculating through a side duct or a duct within the test chamber, and with effectiveness reported as an equivalent clean airflow delivery rate. This can apply to in-duct air cleaning systems that clean the air in an occupied zone.

All air cleaning systems can be tested in-chamber for ozone, formaldehyde, and airborne particulates. Testing for formaldehyde and particulates can be determined concurrently or independently. Formaldehyde testing can be conducted in the presence of a single-pulse injection of limonene resulting in approximately 25 μg/m³ (4.5 ppbv) initial gas concentration in the chamber. The experiment can last at least four hours to allow reactive chemistry to occur. Loss rates for the mitigation systems OFF can be determined according to the procedure described in Stephens et. al 28, or equivalent. The average emission rate for formaldehyde over the test period can be determined using Equation 6:

$$E = V ( {L}_{off} {C}_{t = Δ t} + \frac{{C}_{t = Δ t} - {C}_{t = 0} )}{Δ t} ) \qquad ( 6 )$$

where E=emission rate, μg/h
V=volume of the chamber, m³
L_{off}=first order loss rate for the chemical that includes both air change and surface losses (see Stephens et. al)
C_{t=dt}=concentration at the end of the test period, μg/m³
Cₜ₌₀=concentration at the beginning of the test, μg/m³
Δₜ=total length of test in h; minimum=4

As another reference, ASHRAE Standard 241 describes a technique for determining the equivalent clean airflow of a single occupied space for the purposes of determining infection risk mitigation. The technique utilizes a tracer aerosol (particles) and determines removal based on the tracer aerosol decay rate. The technique utilizes a particle generator that disperses particles into the air of the indoor space from a liquid solution that can generate particles in the E1, E2, and E3 size ranges as defined in ANSI/ASHRAE Standard 52.2. The technique utilizes a particle monitor that measures particle concentrations, with an accuracy of +/−10% of a standard reference. Suitable particle detectors can include instantaneous biological analyzer and collectors for fluorescent-tagged polystyrene beads, qPCR, and DNA sequences for DNA-tagged particles. Example particles can include liquid aerosols such as NaCl, DNA-tagged particles, fluorescent-tagged polystyrene latex beads, and smoke or other particulate-based tracers. The particles should be used in non-toxic and non-sensitizing amounts. A target indoor environment for testing can be up to 900 sqft., though larger areas can be tested by using multiple test areas each up to 900 sqft. to cover the larger space. The technique is valid for 0.35 to 12 air changes per hour (ACH).

The technique calls for:
(1) steady-state conditions for a given ventilation and filtration setup, with HVAC system and mitigation systems settings held constant for the duration of the test;
(2) restricted movement in the test area for the duration of the test,
(3) no other particle generating sources, natural or mechanical, active in the test space;
(4) measured and recorded volume of the space; and

the particle generator is placed in the center of the test area, with at least four particle monitors with at least one device placed in the center of each of four quadrants in the test space, each quadrant having a diameter of up to 15 feet but for spaces smaller than 15×15, the quadrants can be smaller, and the particle generator and particle monitors are placed at the same height within the breathing zone 3 to 72 inches above the floor.

Particle counts are collected for each particle monitor, in E1, E2, and E3, size ranges for the duration of the background, release, and settling periods, which are illustrated in FIG. 25.

The background period can be defined as the period beginning five minutes prior to the particle generator being turned on. Background particle count can be defined as the average particle count in this period.

The release period can be defined as the period beginning when the particle generator is turned on or begins generating particles into the space, and ending when the particle generator turns off or ceases generating particles into the space. Particles are generated until particle counts are at least three times (3×) the background particle count. Depending on the particle monitor and particles being measured, it may be suitable to have high particle counter than three times background; for example, 4×, 5×, 6×, 7×, 8×, 9×, 10×, or greater. The time at which the generator is turned off is t=0 minutes.

The settling period can be defined as the six-minute period beginning when the particle generator is turned off or, alternatively as described herein, when the particle generator ceases generating particles into the space.

The decay period can be defined as the period beginning when the settling period ends and ending after either t=60 minutes or when 90% particle count reduction is reached, whichever comes first.

The collected particle data can be analyzed as follows:
(I) Calculate the target volumetric airflow (V_{ECAi,And,T}) for all particle monitors ADₙ (n=1-4+) using Equation 7 and calculate the target air changes per hour (ACHₜ) using Equation 8.

$$\begin{matrix}{V}_{EACi , ADn , T} = [ ( ECAi \times {P}_{Z , IRMM} ) ] & ( 7 ) \\ {ACH}_{ADn , T} = \frac{( α \times {V}_{EACi , ADn , T} )}{V} & ( 8 )\end{matrix}$$

where
V_{ECAi,ADn,T}=target volumetric airflow per aerosol detector, cfm (L/s)
ECAi=equivalent clean airflow rate required per person, cfm per person (L/s/person)
P_{Z,IRMM}=number of people in the breathing zone
V=volume of the space, ft3 (m3)
ACH_{ADn,T}=target air changes per hour per aerosol detector, h-1
(II) Calculate the global areas under the curve for target (AUC_{T}) and actual (AUC_{A}) as follows:
(II.A) Background actual (calculated as the area under the curve using the average background value extrapolated over the decay period [tSDC to tEDC (A)]).
(II.B) Background target (calculated as the area under the curve using the average background value extrapolated over the target decay period [tSDC to tEDC(T)])
(II.C) The decay period:
(II.C.1). AUCT can be calculated as the area under the exponential decay curve defined by ACHT, minus background target.
(II.C.2). Calculate equation constant a using Equation 9.

$$a = \frac{{PC}_{SDC}}{{e}^{- {ACH}_{I} \times t}} \qquad ( 9 )$$

(II.C.3). Calculate particle concentration (PC) associated with each time step. The last time step is when either the calculated PC is reduced by 90% of the starting value minus the background [10% of (PCSDC−PCBackground)] or t=60 minutes.
(II.C.4). Calculate the area under the curve (Global AUCT) using Equation 10, or by integration between (tSDC, PCSDC) and (tEDC(T), PCEDC).

$$Global {AUC}_{T} = {\sum }_{n = 0}^{n = {t}_{EDC ( T )}} \frac{( {T}_{n + 1} - {t}_{n} ) ( {PC}_{n + 1} + {PC}_{n} )}{2} - {AUC}_{B ( T )} \qquad ( 10 )$$

where:
tSDC=time at the start of the decay curve, min
PCSDC=particle concentration at the start of the decay curve
tEDC(T)=time at the end of the decay curve for the target decay curve, min
PCEDC=particle concentration at the end of the decay curve
n=time steps from 0 to tEDC(T), min
AUCB(T)=area under the curve for the background target
(II.C.5). The actual AUCA (decay period AUC minus background actual):
(II.C.5.i). Calculate the area under the curve (AUCA,ADn,En) for all aerosol detector for each size range using Equation 11 between (tSDC, PCSDC) and (tEDC (A), PCEDC).

$${AUC}_{A , ADn , En} = {\sum }_{n = 0}^{n = {t}_{EDC ( A )}} \frac{( {t}_{n + 1} - {t}_{n} ) ( {PC}_{n + 1} + {PC}_{n} )}{2} - {AUC}_{B ( A )} \qquad ( 11 )$$

(II.C.5.ii). Calculate the weighted average area under the curve using weights WE1, WE2, and WE3 from Section 7.4.1 for all aerosol detectors ADn (n=1-4+) using Equation 12.

$${AUC}_{A , ADn , avg} = {AUC}_{A , ADn , E 1} \times {W}_{E 1} + {AUC}_{A , ADn , E 2} \times {W}_{E 2} + {AUC}_{A , ADn , E 3} \times {W}_{E 3} \qquad ( 12 )$$

Where:
AUCA,ADn,avg=area under the curve for the actual decay, aerosol detector n, averaged over all particle bins (E1, E2, E3)
AUCA,ADn,E1=area under the curve for the actual decay, aerosol detector n,E1
WE1=particle size distribution weighting in the 0.3 to 1.0 micron range, % AUCA,ADn,E2=area under the curve for the actual decay, aerosol detector n,E2
WE2=particle size distribution weighting in the 1.0 to 3.0 micron range, %
AUCA,ADn,E3=area under the curve for the actual decay, aerosol detector n,E3
WE3=particle size distribution weighting in the 3.0 to 10.0 micron range, %
(II.C.5.iii). Calculate the global average (Global AUCA) using Equation 13:

$$Global {AUC}_{A} = \frac{( {AUC}_{A , AD 1 , avg} + {AUC}_{A , AD 2 , avg} + {AUC}_{A , AD 3 , avg} )}{n} \qquad ( 13 )$$

where:
Global AUC_{A}=global average of the area under the curve for all aerosol detectors and size ranges E1, E2, and E3
n=number of aerosol detectors in a test
(III) Calculate the actual volumetric airflow VECAi,ADn,En for each aerosol detector and each E1, E2, and E3 particle size range, using Equation 14.

$${V}_{ECAi , ADn , En} = \frac{{V}_{ECAi , target} \times Global {AUC}_{T}}{{AUC}_{A , ADn , En}} \qquad ( 14 )$$

where
VECAi,ADn,En=volumetric airflow per aerosol detector, actual
VECAi,target=volumetric airflow, target from Equation 6
Global AUCT=area under the curve, target from Equation 7
AUCA,ADn,En=area under the curve, actual from Equation 10
(IV) Calculate the actual weighted average, VECAi,ADn,avg,A for each particle size bin, E1, E2, and E3, and each aerosol detector using the weights WE1, WE2, and WE3 using Equation 15.

$${V}_{ECAi , ADn , avgA} = {V}_{ECAi , ADn , E 1 , A} \times {W}_{E 1} + {V}_{ECAi , ADn , E 2 , A} \times {W}_{E 2 +} {V}_{ECAi , ADn , E 3 , A} \times {W}_{E 3} \qquad ( 15 )$$

where:
VECAi,ADn,avg,A=actual volumetric airflow, aerosol detector n, averaged over all particle bins (E1, E2, E3)
VECAi,ADn,E1,A=particle size distribution weighting in the 0.3 to 1.0 micron range, %
WE1=actual volumetric airflow, aerosol detector n,E1
VECAi,ADn,E2,A=particle size distribution weighting in the 1.0 to 3.0 micron range, %
WE2=actual volumetric airflow, aerosol detector n,E2 VECAi,ADn,E3,A=particle size distribution weighting in the 3.0 to 10.0 micron range, %
WE3=actual volumetric airflow, aerosol detector n,E3
(V) Calculate the target and actual global averages, VECAi,avg,T and VECAi,avg,A, using the average of all the respective values of VECAi,ADn,T and VECAi,ADn,avg,A calculated for the space using Equation 16 and Equation 17.

$$\begin{matrix}{V}_{ECAi , avg , T} = \frac{( {V}_{ECAi , AD 1 , T} + {V}_{ECAi , AD 2 , T} + {V}_{ECAi , AD 3 , T} + … )}{n} & ( 16 ) \\ {V}_{ECAi , avg , A} = \frac{( {V}_{ECAi , AD 1 , avg , A} + {V}_{ECAi , AD 2 , avg , A} + {V}_{ECAi , AD 3 , avg , A} + … )}{n} & ( 17 )\end{matrix}$$

where
VECAi,avg,T=target global average volumetric airflow
n=target global average volumetric airflow
VECAi,avg,A=actual global average volumetric airflow

As illustrated above, the ASHRAE Standard 241 can be used to generate several air characteristics. It also can be used to determine a pass or fail, with a pass defined as an actual global average VECAi,avg,A greater than or equal to the target global average VECAi,avg,T; and a failure defined as an actual global average VECAi,avg,A less than the target global average VECAi,avg,T.

The ASHRAE Standard 241 can also be adapted to monitoring gas in the indoor environment, for example, by relying on a gas generator instead of a particle generator and use of a gas monitor instead of a particle monitor.

Other approaches for monitoring gas characteristics of gas in the indoor environment are also possible. For example, ASTM E741 Standard Test Method for Determining Air Change in a Single Zone by Means of a Tracer Gas Dilution.

As discussed herein above, approaches for assessing air quality and determining particle removal rates can benefit from operatively coupled or time-coordinated gas monitoring, particularly for determining ventilation. Additionally, gas monitoring can be used to determine the volume of air in the indoor environment, which is the effective room size. For example, the air volume of the indoor environment can be readily back calculated from the gas concentration detected in the indoor environment following supply of a known amount of gas when the amount of ventilation is insignificant or by accounting for the amount of any ventilation. As another example, the air volume of the indoor environment can be calculated by using the time to return to baseline after providing a known amount of gas together with using the ventilation rate determined by fitting an exponential to the decay curve of the resulting bolus. This approach can avoid the need for physical measurement of the indoor environment and moreover has the advantage of accounting for objects that reduce the total air volume in the indoor environment. The resulting air volume value can be utilized in each of the various systems and methods described herein whenever volume of the indoor environment is utilized, for example when determining an air characteristic, a particle characteristic, a gas characteristic, particle concentration, particle number, gas concentration, gas number, ACH, MEACH, or CADR values in the above techniques or elsewhere.

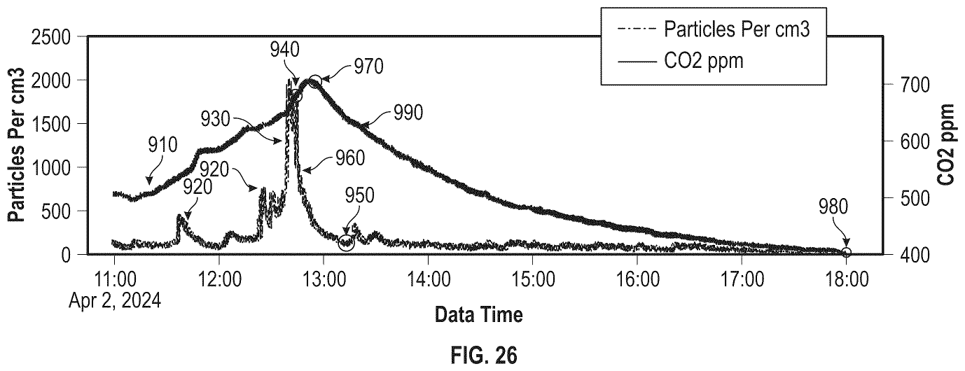

Referring to FIG. 26, example data are provided that illustrate how to determine removal rate based on recording gas or particle characteristics in air of an indoor space. Event 910 indicates the presence of a gas generator, which in this case is a person, which gradually provides CO2 until a maximum concentration around event 970. Event 970 indicates the termination of gas generating activity, which results in a gradual decay period 990 of CO2 until baseline is reached at event 980. Events 920 indicate activities in the indoor space that are generating particles. Event 930 is an event that generates a large amount of particles far above baseline, reaching a maximum or local maximum around event 940. Event 940 also indicates the termination of the particle generating event, which results in gradual decay period 960 of particles until baseline is reached at around event 950. The identified decay periods are useful for determining gas removal rate, particle removal rate, and thus air characteristics of the indoor space and its particle mitigation systems. Gas concentrations were measured during the gas decay phase are processed by fitting an exponential to determine decay rate. Particle concentrations were measured during the gas decay phase are processed by fitting an exponential to determine decay rate. Here, analysis of the decay curves indicated a total particle removal rate of 15 MEACH for the indoor environment overall, and a ventilation rate for the space of 0.6 ACH. Subtracting the ventilation component from the total partial mitigation provided the MEACH for the particle mitigation components of 14.4 MEACH.

Another way to determine removal rates is via continuous monitoring. Here, decay rate can be determined by utilizing a moving time window consisting of a start time and duration, in which collected time series data for particles and/or gas is well fit to an environment modelling equations, such as Equation 3 or Equation 4, and by way of numerical optimization, where the parameters are degrees of freedom for possible window. Additionally, the goodness of fit can be optimized for a time window duration that most closely matches the environment modelling equation for certain determined model parameters. Acceptance of certain windows and parameter values can be defined by required accuracy and statistical power. Threshold trigger can also be used to identify suitable measurement periods. In instances where the concentration, number count, or other measurement has a positive, this can be identified as a region of charge. Once a charge event has been identified as occurring, the data collected compared to a threshold value, and exceeded it is monitored until a decay occurs in which it is brought under threshold. This method is similar in some respects to the method outlined in the AHSREA 241, in which the starting of a decay process must occur at three times the background rate. Other multiples can be suitable as well. Once the decay period has been identified, the curve can be fit using a variety of methods including a moving window style as described above, or using Equation 2, or linear fit methods where in the data is linearized by taking the natural logarithm of the recorded data. This approach can permit continuous and use of historical data processing with out any prior knowledge of charge or decay events within space. In the instance of occupant based generation events, the decay rate can be computed using the data collected once some or all occupants have been identified to have left the space. Again, methods such as a moving window style fit process, Equation 2, other linearization methods can be used, or methods similar to those outlined in ASHREA 241 can be used to compute the decay rates. Decay events can also be captured using calendar and date time specific searches based on historical occupancy information as well. As described herein, using the operatively coupled devices can also be done to identify periods of decay, in which the periods following a known release of particles or gas into the space, for which a charge threshold or other event criteria have been met, such as the charge device having been off for a period of time, identifies the decay period. Again the decay rate can be computed using the moving time window method, Equation 2, linearization methods, ASHREA 241 or other methods in which the decay curve can be accurately computed. Upon an identified decay period, removal rates can be determined as described herein, for example via fitting an exponential or from the area-under-the-curve.

Here, both CO2 and particle concentration are collected over a continuous time period. Various occupant generated particle events occurred within the space, 920 and 940. Events 920 represent common household tasks such as starting a dishwasher and showering, and a cooking activity created the charge and peak particle concentration 940, Using the methods described, event 940 indicates beginning of a decay curve period, during which particles reach a maximum or local maximum concentration well above the background. Subsequent concentration decay suggest this as a period of decay with sufficient magnitude to resolve the indoor environment mitigation equivalent air change rate, CADR or other relevant metric determined from the decay rate. The CO2 monitored curve indicates the introduction of a CO2 generator around within the room space, which provides a steady increase 910 that represents the occupant generating CO2 within the space, with slight variation in slope due to the CO2 generation rate for the occupant changing due to physical activity. The maximum or local maximum concentration 970 occurs when the occupant leaves the space, removing all sources of CO2 as the room becomes unoccupied. At this point, CO2 levels are well above well above the background, afterwhich, the CO2 levels begin to decay. The decay region, 990, can be identified and computed using prior described methods and continues until the CO2 returns to baseline.

The following Examples are provided as example aspects of the disclosed subject matter:

Example 1 provides a system for analyzing air of an indoor environment having a plurality of particle mitigation systems including at least a mechanical air filtration system having a particle filtration profile wherein a first particle size is filtered less than a second particle size and an air handling system for exchanging air with an outdoor environment is provided. The system comprising a particle generator configured to charge the indoor environment with a first charge including a concentration of a plurality of test particles; a particle monitor configured to receive air from the indoor environment to detect the plurality of test particles; a gas generator configured to charge the indoor environment with a second charge including a concentration of a test gas; a gas monitor configured to receive air from the indoor environment to detect the test gas; and a control system operatively coupled to the particle monitor and the gas monitor to determine at least one air characteristic of the indoor environment based on a test gas characteristic related to a first portion of the particle mitigation systems of the indoor environment and a test particle characteristic related to a second portion of the particle mitigation systems of the indoor environment. The plurality of test particles having a test particle size about equal to the first particle size. The test gas having a size less than the test particle size.

Example 2 provides a system for analyzing air of an indoor environment having a plurality of particle mitigation systems including at least a mechanical air filtration system having a particle filtration profile wherein a first particle size is filtered less than a second particle size, an air handling system for exchanging air with an outdoor environment, and a light system is provided. The system comprising a first monitor operable to characterize the air handling system; a second monitor operable to characterize the mechanical air filtration system; a third monitor operable to characterize the light system; and a control system operatively coupled to the first monitor, the second monitor, and the third monitor to determine at least one air characteristic of the indoor environment.

Example 3 provides a system for analyzing air of an indoor environment having at least one particle mitigation system is provided. The at least one particle mitigation system receiving air from an outdoor environment and filtering air which has been circulated in the indoor environments with at least one filter having a particle filtration profile wherein a first particle size is filtered less than a second particle size. The system comprising a particle generator configured to charge the indoor environment with a first charge including a concentration of a plurality of test particles; a particle monitor configured to receive air from the indoor environment to detect the test particle during a first time window; a gas generator configured to charge the indoor environment with a second charge including a concentration of a test gas; a gas monitor configured to receive air from the indoor environment to detect the test gas during the first time window; and a control system operatively coupled to the particle monitor and the gas monitor to determine at least one air characteristic of the indoor environment. The plurality of test particles having a test particle size about equal to the first particle size. The test gas having a size less than the test particle size.

Example 4 provides a system of Example 1, wherein the control system based on the test gas characteristic determines a first air handling system characteristic.

Example 5 provides a system of any one of Examples 1-2, wherein the control system based on the test particle characteristic determines a first mechanical filtering characteristic.

Example 6 provides a system of Example 5, wherein the first mechanical filtering characteristic is further based on the test gas characteristic.

Example 7 provides a system of Examples 1-6, wherein the at least one air characteristic of the indoor environment includes a mitigation equivalent air change rate (MEACH) for the plurality of particle mitigation systems.

Example 8 provides a system of Examples 1-7, wherein the control system based on the at least one air characteristic of the indoor environment provides an alert with an output device.

Example 9 provides a system of Example 8, wherein the output device is one of a visual output device, an audio output device, and a network device.

Example 10 provides a system of Examples 1-9, wherein the control system based on the at least one air characteristic of the indoor environment alters at least one setting of the plurality of particle mitigation systems.

Example 11 provides a system of Examples 1-9, wherein the control system based on the at least one air characteristic of the indoor environment alters at least one setting of the gas generator.

Example 12 provides a system of Examples 1-11, wherein the plurality of test particles have a number mean particle diameter in the range of about 0.03 microns to about 0.3 microns.

Example 13 provides a system of Examples 1-11, wherein the plurality of test particles have a number mean particle diameter in the range of about 0.06 microns to about 0.3 microns.

Example 14 provides a system of Examples 1-11, wherein the plurality of test particles have a number mean particle diameter in the range of about 0.03 microns to about 1.0 microns.

Example 15 provides a system of Examples 1-14, wherein the plurality of test particles are optically reactive to at least one of the plurality of particle mitigation systems.

Example 16 provides a system of Example 15, wherein the plurality of test particles have a first spectral response when exposed to UV light from a first one of the plurality of particle mitigation systems and a second spectral response when not exposed to UV light from the first one of the plurality of particle mitigation systems.

Example 17 provides a system for analyzing air of an indoor environment having a plurality of particle mitigation systems, the system comprising:
a particle monitor configured to receive air having particles generated by a particle generator in the indoor environment and monitor a particle characteristic over time;
a gas monitor configured to receive air having a gas generated by a gas generator in the indoor environment and monitor a gas characteristic over time; and
a control system operatively coupled to the particle monitor and the gas monitor to determine at least one air characteristic of the indoor environment based on both the particle characteristic and the gas characteristic.

Example 18 provides a system of Example-1, wherein the at least one air characteristic of the indoor environment includes a mitigation equivalent air change rate (MEACH) for a portion of the particle mitigation systems based on both the particle characteristic and the gas characteristic.

Example 19 provides a system of any one of Examples 17-1, wherein the at least one air characteristic of the indoor environment includes a mitigation equivalent air change rate (MEACH) for non-ventilation portions of the plurality particle mitigation systems based on both the particle characteristic and the gas characteristic.

Example 20 provides a system of any one of Examples 17-1, wherein the plurality of particle mitigation systems includes a mechanical air filtration system, and the at least one air characteristic of the indoor environment includes a mitigation equivalent air change rate (MEACH) for a mechanical air filtration system portion of the plurality of particle mitigation systems based on both the particle characteristic and the gas characteristic.

Example 21 provides a system of any one of Examples 17-1, wherein the plurality of particle mitigation systems includes a mechanical air filtration system and an air handling system providing ventilation, and the control system is configured for determining a mitigation equivalent air change rate (MEACH) for the mechanical air filtration system and an air change rate (ACH) for the air handling system.

Example 22 provides a system of any one of Examples 17-1, wherein the plurality of particle mitigation systems includes a mechanical air filtration system and an air handling system providing ventilation, and the control system is configured for determining a mitigation equivalent air change rate (MEACH) for the mechanical air filtration system, an air change rate (ACH) for all ventilation for the indoor environment.

Example 23 provides a system of any one of Examples 17-1, wherein the control system determines a mitigation equivalent air change rate (MEACH) for the indoor environment based on all particle mitigation systems and determines an air change rate (ACH) for all ventilation.

Example 24 provides a system of any one of Examples 17-1, wherein the control system determines a clean air delivery rate (CADR) for the indoor environment overall based on both the particle characteristic and the gas characteristic.

Example 25 provides a system of any one of Examples 17-1, wherein the control system determines a clean air delivery rate (CADR) for the indoor environment overall based on combining the mitigation equivalent air change rate (MEACH) for all particle mitigation systems and the air change rate (ACH) for all ventilation.

Example 26 provides a system of any one of Examples 17-1, wherein the plurality of particle mitigation systems includes a mechanical air filtration system, and the control system determines a clean air delivery rate (CADR) for the indoor environment overall based on combination of the mitigation equivalent air change rate (MEACH) for the mechanical air filtration system and the air change rate (ACH) for all ventilation.

Example 27 provides a system of any one of Examples 17-1, wherein the particle characteristic and the gas characteristic are measured simultaneously or contemporaneously.

Example 28 provides a system of any one of Examples 17-1, wherein the one or more air characteristic of the indoor environment is time resolved based on the gas characteristic.

Example 29 provides a system of any one of Examples 17-1, wherein the control system is configured to determine a volume for the indoor environment based on the gas characteristic together with an estimated or known amount of gas provided by the gas generator.

Example 30 provides a system of any one of Examples 17-1, wherein the control system is configured to utilizes a volume for the indoor environment determined from the gas characteristic when determining the one or more air characteristic of the indoor environment.

Example 31 provides a system of any one of Examples 17-1, wherein the one or more air characteristic of the indoor environment is determined approximately real-time relative to monitoring the gas characteristics.

Example 32 provides a system of any one of Examples 17-1, wherein: the gas includes two or more component gases each having differing interactions with one or more portions of the particle mitigation systems;
the gas monitor is configured to monitor gas characteristics of the two or more component gases over time; and
the control system is configured to determine a mitigation equivalent air change rate (MEACH) for the one or more portions of the particle mitigation systems.

Example 33 provides a system of any one of Examples 17-1, wherein the plurality of particle mitigation systems includes a scrubber:
the gas includes two or more component gases each having differing interactions with the scrubber;
the gas monitor is configured to monitor gas characteristics of the two or more component gases over time; and
the control system is configured to determine a mitigation equivalent air change rate (MEACH) for the scrubber.

Example 34 provides a system of any one of Examples 17-1, wherein the control system is configured for:
estimating volume of the indoor environment based on a gas release characteristic of the gas generator and recording gas concentration in the indoor environment over time while the indoor environment is charging with the gas; and
determining outdoor environment ventilation air change rate based on recording gas concentration in the indoor environment over time after the indoor environment is charged with the gas.

Example 35 provides a system of any one of Examples 17-1, wherein the control system is configured for:
determining indoor environment mitigation equivalent air change rate based on recording gas concentration in the indoor environment over time after the indoor environment is charged with the gas.

Example 36 provides a system of any one of Examples 17-1, wherein the control system is configured for:
determining indoor environment mitigation equivalent air change rate based on both recording gas concentration in the indoor environment over time after the indoor environment is charged with the gas and recording particle concentration in the indoor environment over time after the indoor environment is charged with the particles.

Example 37 provides a system of any one of Examples 17-1, wherein the control system is configured for:
assessing at least one air characteristic of the indoor environment against a air characteristic criteria; and
altering a setting of one or more portion of the plurality of particle mitigation systems, a mechanical air filtration system, an air handling system, providing an alert, or any combination thereof.

Example 38 provides a system of any one of Examples 17-1, wherein the control system is configured for:
determining a change in occupancy and/or occupant activity of the indoor environment based on the gas characteristic gas.

Example 39 provides a system of any one of Examples 17-1, wherein the control system is configured for:
determining a change in occupant activity of the indoor environment based on the particle characteristic gas.

Example 40 provides a system of any one of Examples 17-1, wherein the control system is configured for:
determining a change in occupancy or occupant activity of the indoor environment by utilizing both an outdoor environment ventilation air change rate based on recording gas concentration in the indoor environment over time and an indoor environment mitigation equivalent air change rate based on recording gas concentration in the indoor environment over time.

Example 41 provides a system of any one of Examples 17-1, wherein the control system is configured for:
determining at least one air characteristic of the indoor environment based on utilizing occupant number, occupant activity, or both, together with an expected CO2 production rate and a measured gas decay rate determined from recording gas concentration in the indoor environment over time.

Example 42 provides a system of any one of Examples 17-1, wherein the control system is configured for:
determining at least one air characteristic of the indoor environment based on utilizing occupant activity together with an expected particle production rate and a measured particle decay rate determined from recording particle concentration in the indoor environment over time.

Example 43 provides a system of any one of Examples 17-1, wherein the control system is configured for:
determining a mitigation equivalent air change rate (MEACH) for one or more portion of the particle mitigation systems based on utilizing occupant number, occupant activity, or both, together with an expected CO2 production rate and a measured gas decay rate determined from recording gas concentration in the indoor environment over time.

Example 44 provides a system of any one of Examples 17-1, wherein the control system is configured for:
determining a mitigation equivalent air change rate (MEACH) for one or more portion of the particle mitigation systems based on utilizing occupant number, occupant activity, or both, together with an expected particle production rate and a measured particle decay rate determined from recording particle concentration in the indoor environment over time.

Example 45 provides a system of any one of Examples 17-1, wherein the plurality of particle mitigation systems includes a mechanical air filtration system, and the control system determines at least one air characteristic of the indoor environment based on a characteristic of the mechanical air filtration system.

Example 46 provides a system of any one of Examples 17-1, wherein the plurality of particle mitigation systems includes an air handling system, and the control system determines at least one air characteristic of the indoor environment based on a characteristic of the air handling system.

Example 47 provides a system of any one of Examples 17-1, wherein the plurality of particle mitigation systems includes a mechanical air filtration system and an air handling system, and the control system determines a characteristic of the mechanical air filtration system and a characteristic of the air handling system.

Example 48 provides a system of any one of Examples 17-1, wherein the plurality of particle mitigation systems includes a light system, and the control system determines a characteristic of the light system.

Example 49 provides a system of any one of Examples 17-1, wherein the plurality of particle mitigation systems includes a scrubber, and the control system determines a characteristic of the scrubber.

Example 50 provides a system of any one of Examples 17-1, wherein the plurality of particle mitigation systems includes a mechanical air filtration system, and the control system determines a mitigation equivalent air change rate (MEACH) for the mechanical air filtration system.

Example 51 provides a system of any one of Examples 17-1, wherein the plurality of particle mitigation systems includes an air handling system, and the control system determines a mitigation equivalent air change rate (MEACH) for the air handling system.

Example 52 provides a system of any one of Examples 17-1, wherein the plurality of particle mitigation systems includes a light system, and the control system determines a mitigation equivalent air change rate (MEACH) for the light system.

Example 53 provides a system of any one of Examples 17-1, wherein the plurality of particle mitigation systems includes a scrubber, and the control system determines a mitigation equivalent air change rate (MEACH) for the scrubber.

Example 54 provides a system of any one of Examples 17-1, wherein the control system determines at least one air characteristic of the indoor environment based on occupancy of the indoor environment, activity of occupants of the indoor environment, or both.

Example 55 provides a system of any one of Examples 17-1, wherein the control system determines whether gas concentration is at a steady state least one air characteristic of the indoor environment

Example 56 provides a system of any one of Examples 17-1, wherein the particle characteristic is particle concentration in the air of the indoor environment.

Example 57 provides a system of any one of Examples 17-1, wherein the particle characteristic describes how particle concentration is changing at given time.

Example 58 provides a system of any one of Examples 17-1, wherein the particle characteristic is a steady state, a charging state, or a decay state. 59 provides a system of any one of Examples 17-1, wherein the gas characteristic is gas concentration in the air of the indoor environment.

Example 60 provides a system of any one of Examples 17-1, wherein the gas characteristic describes how gas concentration is changing at given time.

Example 61 provides a system of any one of Examples 17-1, wherein the gas characteristic is a steady state, a charging state, or a decay state.

Example 62 provides a system of any one of Examples 17-1, wherein the gas comprises CO2.

Example 63 provides a system of any one of Examples 17-1, wherein the gas comprises isopropanol.

Example 64 provides a system of any one of Examples 17-63, wherein the amount of gas provided to the indoor environment is a known, measured, or metered amount of gas.

Example 65 provides a system of any one of Examples 17-1, wherein the gas is generated from a container.

Example 66 provides a system of any one of Examples 17-1, wherein the gas is generated from one or more people present in the indoor environment.

Example 67 provides a system of any one of Examples 17-1, wherein the gas generator comprises a sensor for determining occupancy or occupant activity in the indoor environment, and occupants or occupant activity in the indoor environment generate the gas and charge the indoor environment.

Example 68 provides a system of any one of Examples 17-1, wherein the particles comprise sodium chloride, oil, fluorescent material, genetic material, a model particle of viral particles, or a combination thereof.

Example 69 provides a system of any one of Examples 17-1, wherein the particles comprise non-peptide fluorophores comprising DNA.

Example 70 provides a system of any one of Examples 17-1, wherein the particles comprise a mineral salt.

Example 71 provides a system of any one of Examples 17-1, wherein the particles are generated from a container.

Example 72 provides a system of any one of Examples 17-1, wherein the particles are provided by a collision atomizer, an ultrasonic atomizer, a candle, a combustion based source, or a vaporization device.

Example 73 provides a system of any one of Examples 17-1, wherein the particles are generated from one or more occupant present in the indoor environment.

Example 74 provides a system of any one of Examples 17-1, wherein the particles are generated from a combustion source.

Example 75 provides a system of any one of Examples 17-1, wherein the particles are generated from a stove, oven, or cooking-based human activity.

Example 76 provides a system of any one of Examples 17-1, wherein the particles and the gas are generated from a container that provides both the particles and the gas.

Example 77 provides a system of any one of Examples 17-1, wherein the particle generator comprises a sensor for determining occupancy or occupant activity in the indoor environment, and occupants or occupant activity in the indoor environment generate the particles and charges the indoor environment.

Example 78 provides a system of any one of Examples 17-1, wherein the particle monitor passively monitors the particle characteristics to identify: initiation of a particle generating event, termination of a particle generation event, achievement of a particle steady state, or any combination thereof.

Example 79 provides a system of any one of Examples 17-1, wherein the particle monitor passively monitors the particle characteristics to identify a background, a signal-to-background threshold, or both.

Example 80 provides a system of any one of Examples 17-1, wherein the gas monitor passively monitors the gas characteristic to identify: initiation of a gas generating event, termination of a gas generation event, achievement of a gas steady state, or any combination thereof.

Example 81 provides a system of any one of Examples 17-1, wherein the gas monitor passively monitors the particle characteristics to identify a background, a signal-to-background threshold, or both.

Example 82 provides a system of any one of Examples 17-81, which is configured to provide a known, measured, or metered amount of gas to the air of the indoor environment and the control system is configured to determine a volume of the air of the indoor environment from utilizing a measured concentration of the gas with the known, measured, or metered amount of gas provided.

Example 83 provides a system of any one of Examples 17-82, wherein the gas characteristic, particle characteristic, or air characteristic is calculated based on using a volume of the air of the indoor environment determined from utilizing a measured concentration of gas in the air with a known, measured, or metered amount of gas provided to the air.

Example 84 provides a system of any one of Examples 17-83, which is configured to provide a known, measured, or metered amount of particles to the air of the indoor environment and the control system is configured to determine a volume of the air of the indoor environment from utilizing a measured concentration of the particles with the known, measured, or metered amount of particles provided.

Example 85 provides a system of any one of Examples 17-84, wherein the particles characteristic, particle characteristic, or air characteristic is calculated based on using a volume of the air of the indoor environment determined from utilizing a measured concentration of particles in the air with a known, measured, or metered amount of particles provided to the air.

Example 86 provides a system of any one of Examples 17-85, which is configured to determine the volume of the air in the indoor environment.

Example 87. A system for analyzing air of an indoor environment having a plurality of particle mitigation systems, the system comprising:
a data input configured to receive a mitigation equivalent air change rate (MEACH) for the plurality of particle mitigation systems, or one or more portion thereof;
a gas monitor configured to receive air having a gas generated by a gas generator in the indoor environment and monitor a gas characteristic over time; and
a control system operatively coupled to the gas monitor to determine at least one air characteristic of the indoor environment based on both the gas characteristic and the mitigation equivalent air change rate (MEACH).

Example 88. A system for analyzing air of an indoor environment having a plurality of particle mitigation systems, the system comprising:
a particle monitor configured to receive air having particles generated by occupant activity in the indoor environment and monitor a particle characteristic over time;
a gas monitor configured to receive air having a gas generated by occupants in the indoor environment and monitor a gas characteristic over time; and
a control system operatively coupled to the particle monitor and the gas monitor to determine at least one air characteristic of the indoor environment based on both the particle characteristic and the gas characteristic.

Example 89. A system for passively analyzing air of an indoor environment having a plurality of particle mitigation systems, the system comprising:
a particle monitor configured to:
receive air having particles generated by occupant activity in the indoor environment,
passively monitor to identify a suitable gas measurement period for determining a particle characteristic over time,
record the particle characteristic during the suitable gas measurement period;
a gas monitor configured to:
receive air having gas generated by a occupants in the indoor environment,
passively monitor to identify a suitable gas measurement period for determining a gas characteristic over time, record the gas characteristic during the suitable gas measurement period;
a control system operatively coupled to the particle monitor and the gas monitor to determine at least one air characteristic of the indoor environment based on the recorded particle characteristic and the recorded gas characteristic.

Example 90 provides a method for analyzing air of an indoor environment having a plurality of particle mitigation systems, the method comprising:
monitoring over time a particle characteristic of air having particles generated by a particle generator in the indoor environment;
monitoring over time a gas characteristic of air having gas generated by a gas generator in the indoor environment;
determining at least one air characteristic of the indoor environment based on both the particle characteristic and the gas characteristic, wherein the monitoring of the particle characteristic and the monitoring of the gas characteristic are time-coordinated.

Example 91 provides a method of Example-1, wherein the at least one air characteristic of the indoor environment includes a mitigation equivalent air change rate (MEACH) for a portion of the particle mitigation systems based on both the particle characteristic and the gas characteristic.

Example 92 provides a method of any one of Examples 90-1, wherein the at least one air characteristic of the indoor environment includes a mitigation equivalent air change rate (MEACH) for non-ventilation portions of the plurality particle mitigation systems based on both the particle characteristic and the gas characteristic.

Example 93 provides a method of any one of Examples 90-1, wherein the plurality of particle mitigation systems includes a mechanical air filtration system, and the at least one air characteristic of the indoor environment includes a mitigation equivalent air change rate (MEACH) for a mechanical air filtration system portion of the plurality of particle mitigation systems based on both the particle characteristic and the gas characteristic.

Example 94 provides a method of any one of Examples 90-1, wherein the plurality of particle mitigation systems includes a mechanical air filtration system and an air handling system providing ventilation, and comprising determining a mitigation equivalent air change rate (MEACH) for the mechanical air filtration system and an air change rate (ACH) for the air handling system.

Example 95 provides a method of any one of Examples 90-1, wherein the plurality of particle mitigation systems includes a mechanical air filtration system and an air handling system providing ventilation, and comprising determining a mitigation equivalent air change rate (MEACH) for the mechanical air filtration system, an air change rate (ACH) for all ventilation for the indoor environment.

Example 96 provides a method of any one of Examples 90-1, comprising determining a mitigation equivalent air change rate (MEACH) for the indoor environment based on all particle mitigation systems and determines an air change rate (ACH) for all ventilation.

Example 97 provides a method of any one of Examples 90-1, comprising determining a clean air delivery rate (CADR) for the indoor environment overall based on both the particle characteristic and the gas characteristic.

Example 98 provides a method of any one of Examples 90-1, comprising determining a clean air delivery rate (CADR) for the indoor environment overall based on combining the mitigation equivalent air change rate (MEACH) for all particle mitigation systems and the air change rate (ACH) for all ventilation.

Example 99 provides a method of any one of Examples 90-1, wherein the plurality of particle mitigation systems includes a mechanical air filtration system, and comprising determining a clean air delivery rate (CADR) for the indoor environment overall based on combination of the mitigation equivalent air change rate (MEACH) for the mechanical air filtration system and the air change rate (ACH) for all ventilation.

Example 100 provides a method of any one of Examples 90-1, wherein the particle characteristic and the gas characteristic are measured simultaneously or contemporaneously.

Example 101 provides a method of any one of Examples 90-1, wherein the one or more air characteristic of the indoor environment is time resolved based on the gas characteristic.

Example 102 provides a method of any one of Examples 90-1, comprising determining a volume for the indoor environment based on the gas characteristic together with an estimated or known amount of gas provided by the gas generator.

Example 103 provides a method of any one of Examples 90-1, comprising utilizing a volume for the indoor environment determined from the gas characteristic when determining the one or more air characteristic of the indoor environment.

Example 104 provides a method of any one of Examples 90-1, wherein the one or more air characteristic of the indoor environment is determined approximately real-time relative to monitoring the gas characteristics.

Example 105 provides a method of any one of Examples 90-1, wherein the gas includes two or more component gases each having differing interactions with one or more portions of the particle mitigation systems, and the method further comprising:
monitoring over time gas characteristics of the two or more component gases over time; and
determining a mitigation equivalent air change rate (MEACH) for a portions of the particle mitigation systems based on the differences in the gas characteristics of the two or more component gases over time.

Example 106 provides a method of any one of Examples 90-1, wherein the plurality of particle mitigation systems includes a scrubber and the gas includes two or more component gases each having differing interactions with the scrubber, and the method further comprising:
monitoring over time gas characteristics of the two or more component gases over time; and
determining a mitigation equivalent air change rate (MEACH) for a portions of the particle mitigation systems based on the differences in the gas characteristics of the two or more component gases over time.

Example 107 provides a method of any one of Examples 90-1, comprising:
estimating volume of the indoor environment based on a gas release characteristic of the gas generator and recording gas concentration in the indoor environment over time while the indoor environment is charging with the gas; and
determining outdoor environment ventilation air change rate based on recording gas concentration in the indoor environment over time after the indoor environment is charged with the gas.

Example 108 provides a method of any one of Examples 90-1, comprising:
determining indoor environment mitigation equivalent air change rate based on recording gas concentration in the indoor environment over time after the indoor environment is charged with the gas.

Example 109 provides a method of any one of Examples 90-1, comprising:
determining indoor environment mitigation equivalent air change rate based on both recording gas concentration in the indoor environment over time after the indoor environment is charged with the gas and recording particle concentration in the indoor environment over time after the indoor environment is charged with the particles.

Example 110 provides a method of any one of Examples 90-1, comprising:
assessing at least one air characteristic of the indoor environment against a air characteristic criteria; and
altering a setting of one or more portion of the plurality of particle mitigation systems, a mechanical air filtration system, an air handling system, providing an alert, or any combination thereof.

Example 111 provides a method of any one of Examples 90-1, comprising:
determining a change in occupancy and/or occupant activity of the indoor environment based on the gas characteristic gas.

Example 112 provides a method of any one of Examples 90-1, comprising:
determining a change in occupant activity of the indoor environment based on the particle characteristic gas.

Example 113 provides a method of any one of Examples 90-1, comprising:
determining a change in occupancy or occupant activity of the indoor environment by utilizing both an outdoor environment ventilation air change rate based on recording gas concentration in the indoor environment over time and an indoor environment mitigation equivalent air change rate based on recording gas concentration in the indoor environment over time.

Example 114 provides a method of any one of Examples 90-1, comprising:
determining at least one air characteristic of the indoor environment based on utilizing occupant number, occupant activity, or both, together with an expected CO2 production rate and a measured gas decay rate determined from recording gas concentration in the indoor environment over time.

Example 115 provides a method of any one of Examples 90-1, comprising:
determining at least one air characteristic of the indoor environment based on utilizing occupant activity together with an expected particle production rate and a measured particle decay rate determined from recording particle concentration in the indoor environment over time.

Example 116 provides a method of any one of Examples 90-1, comprising:
determining a mitigation equivalent air change rate (MEACH) for one or more portion of the particle mitigation systems based on utilizing occupant number, occupant activity, or both, together with an expected CO2 production rate and a measured gas decay rate determined from recording gas concentration in the indoor environment over time.

Example 117 provides a method of any one of Examples 90-1, comprising:
determining a mitigation equivalent air change rate (MEACH) for one or more portion of the particle mitigation systems based on utilizing occupant number, occupant activity, or both, together with an expected particle production rate and a measured particle decay rate determined from recording particle concentration in the indoor environment over time.

Example 118 provides a method of any one of Examples 90-1, wherein the plurality of particle mitigation systems includes a mechanical air filtration system, and the control system determines at least one air characteristic of the indoor environment based on a characteristic of the mechanical air filtration system.

Example 119 provides a method of any one of Examples 90-1, wherein the plurality of particle mitigation systems includes an air handling system, and the control system determines at least one air characteristic of the indoor environment based on a characteristic of the air handling system.

Example 120 provides a method of any one of Examples 90-1, wherein the plurality of particle mitigation systems includes a mechanical air filtration system and an air handling system, and the control system determines a characteristic of the mechanical air filtration system and a characteristic of the air handling system.

Example 121 provides a method of any one of Examples 90-1, wherein the plurality of particle mitigation systems includes a light system, and the control system determines a characteristic of the light system.

Example 122 provides a method of any one of Examples 90-1, wherein the plurality of particle mitigation systems includes a scrubber, and the control system determines a characteristic of the scrubber.

Example 123 provides a method of any one of Examples 90-1, wherein the plurality of particle mitigation systems includes a mechanical air filtration system, and the control system determines a mitigation equivalent air change rate (MEACH) for the mechanical air filtration system.

Example 124 provides a method of any one of Examples 90-1, wherein the plurality of particle mitigation systems includes an air handling system, and the control system determines a mitigation equivalent air change rate (MEACH) for the air handling system.

Example 125 provides a method of any one of Examples 90-1, wherein the plurality of particle mitigation systems includes a light system, and the control system determines a mitigation equivalent air change rate (MEACH) for the light system.

Example 126 provides a method of any one of Examples 90-1, wherein the plurality of particle mitigation systems includes a scrubber, and the control system determines a mitigation equivalent air change rate (MEACH) for the scrubber.

Example 127 provides a method of any one of Examples 90-1, wherein the control system determines at least one air characteristic of the indoor environment based on occupancy of the indoor environment, activity of occupants of the indoor environment, or both.

Example 128 provides a method of any one of Examples 90-1, wherein the control system determines whether gas concentration is at a steady state least one air characteristic of the indoor environment

Example 129 provides a method of any one of Examples 90-1, wherein the particle characteristic is particle concentration in the air of the indoor environment.

Example 130 provides a method of any one of Examples 90-1, wherein the particle characteristic describes how particle concentration is changing at given time.

Example 131 provides a method of any one of Examples 90-1, wherein the particle characteristic is a steady state, a charging state, or a decay state. 132 provides a method of any one of Examples 90-1, wherein the gas characteristic is gas concentration in the air of the indoor environment.

Example 133 provides a method of any one of Examples 90-1, wherein the gas characteristic describes how gas concentration is changing at given time.

Example 134 provides a method of any one of Examples 90-1, wherein the gas characteristic is a steady state, a charging state, or a decay state.

Example 135 provides a method of any one of Examples 90-1, wherein the gas comprises CO2.

Example 136 provides a method of any one of Examples 90-1, wherein the gas comprises isopropanol.

Example 137 provides a method of any one of Examples 90-1, wherein the gas is generated from a container.

Example 138 provides a method of any one of Examples 90-137, wherein the amount of gas provided to the indoor environment is a known, measured, or metered amount of gas

Example 139 provides a method of any one of Examples 90-1, wherein the gas is generated from one or more people present in the indoor environment.

Example 140 provides a method of any one of Examples 90-1, wherein the gas generator comprises a sensor for determining occupancy or occupant activity in the indoor environment, and occupants or occupant activity in the indoor environment generate the gas and charge the indoor environment.

Example 141 provides a method of any one of Examples 90 provides a method of any one of Examples 90-1, wherein the particles comprise sodium chloride, oil, fluorescent material, genetic material, a model particle of viral particles, or a combination thereof.

Example 142 provides a method of any one of Examples 90-1, wherein the particles comprise non-peptide fluorophores comprising DNA.

Example 143 provides a method of any one of Examples 90-1, wherein the particles comprise a mineral salt.

Example 144 provides a method of any one of Examples 90-1, wherein the particles are generated from a container.

Example 145 provides a method of any one of Examples 90-1, wherein the particles are provided by a collison atomizer, an ultrasonic atomizer, a candle, a combustion based source, or a vaporization device.

Example 146 provides a method of any one of Examples 90-1, wherein the particles are generated from one or more occupant present in the indoor environment.

Example 147 provides a method of any one of Examples 90-1, wherein the particles are generated from a combustion source.

Example 148 provides a method of any one of Examples 90-1, wherein the particles are generated from a stove, oven, or cooking-based human activity.

Example 149 provides a method of any one of Examples 90-1, wherein the particles and the gas are generated from a container that provides both the particles and the gas.

Example 150 provides a method of any one of Examples 90-1, wherein the particle generator comprises a sensor for determining occupancy or occupant activity in the indoor environment, and occupants or occupant activity in the indoor environment generate the particles and charges the indoor environment.

Example 151 provides a method of any one of Examples 90-1, comprising passively monitoring the particle characteristic of the air in the indoor environment to identify: initiation of a particle generating event, termination of a particle generation event, achievement of a particle steady state, or any combination thereof.

Example 152 provides a method of any one of Examples 90-1, comprising passively monitoring the particle characteristic of the air in the indoor environment to identify a background, a signal-to-background threshold, or both.

Example 153 provides a method of any one of Examples 90-1, comprising passively monitoring the gas characteristic of the air in the indoor environment to identify: initiation of a gas generating event, termination of a gas generation event, achievement of a gas steady state, or any combination thereof.

Example 154 provides a method of any one of Examples 90-1, comprising passively monitoring the gas characteristic of the air in the indoor environment to identify: a background, a signal-to-background threshold, or both.

Example 155 provides a method of any one of Examples 90-154, comprising providing a known, measured, or metered amount of gas to the air of the indoor environment and determining a volume of the air of the indoor environment by utilizing a measured concentration of the gas with the known, measured, or metered amount of gas provided.

Example 156 provides a method of any one of Examples 90-155, wherein the gas characteristic, particle characteristic, or air characteristic is determined based on using a volume of the air of the indoor environment determined from utilizing a measured concentration of gas in the air with a known, measured, or metered amount of gas provided to the air.

Example 157 provides a method of any one of Examples 90-156, comprising determining a volume of the air of the indoor environment by utilizing a measured concentration of particles provided by a known, measured, or metered bolus.

Example 158 provides a method of any one of Examples 90-157, wherein the particles characteristic, particle characteristic, or air characteristic is calculated based on using a volume of the air of the indoor environment determined from utilizing a measured concentration of particles in the air with a known, measured, or metered amount of particles provided to the air.

Example 159 provides a method of any one of Examples 90-158, comprising determining the volume of the air in the indoor environment without physically measuring dimensions of the indoor environment.

Example 160 provides a method for analyzing air of an indoor environment having a plurality of particle mitigation systems, the method comprising:
providing a mitigation equivalent air change rate (MEACH) for the plurality of particle mitigation systems, or one or more portion thereof;
monitoring over time a gas characteristic of air having gas generated by a gas generator in the indoor environment;
determining at least one air characteristic of the indoor environment based on both the gas characteristic and the mitigation equivalent air change rate (MEACH).

Example 161 provides a method for analyzing air of an indoor environment having a plurality of particle mitigation systems, the method comprising:
monitoring over time a particle characteristic of air having particles generated by occupant activity in the indoor environment;
monitoring over time a gas characteristic of air having a gas generated by occupants in the indoor environment;
determining at least one air characteristic of the indoor environment based on both the particle characteristic and the gas characteristic.

Example 162 provides a method for passively analyzing air of an indoor environment having a plurality of particle mitigation systems, the method comprising:
monitoring over time a particle characteristic of air having particles generated by occupant activity in the indoor environment;
monitoring over time a gas characteristic of air having particles generated by occupants in the indoor environment;
determining a suitable particle measurement period based on analyzing the particle characteristics over time;
determining a suitable gas measurement period based on analyzing the gas characteristic over time;
recording the particle characteristic during the suitable particle measurement period;
recording the gas characteristic during the suitable particle measurement period;
determining at least one air characteristic of the indoor environment based on both the recorded particle characteristic and the recorded gas characteristic.

Example 163 provides a method for analyzing air of an indoor environment having a plurality of particle mitigation systems including a mechanical air filtration system and an air handling system, the method comprising:
charging the indoor environment with particles comprising NaCl, non-peptide fluorophores, fluorescent tagged proteins, or a mixture thereof;
charging the indoor environment with a gas comprising a volatile organic compound;
monitoring over time a particle characteristic of air having the particles in the indoor environment;
monitoring over time a gas characteristic of air having the gas in the indoor environment; and
determining at least one air characteristic of the indoor environment based on the particle characteristic and the gas characteristic.

Example 164 system configured for performing the method of any one of Examples 90-163.

Example 165 system comprising a particle monitor, a gas monitor, and a control system, configured for performing the method of any one of Examples 90-164.

Example 166 system comprising a particle monitor, a gas monitor, a means for gas generation, a means for particle generation, and a control system, configured for performing the method of any one of Examples 90-165.

Example 167 provides a method or system incorporated any combination or permutation of one or more of the aforementioned features in Examples 1-166.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A method for analysing air of an indoor environment having a plurality of particle mitigation systems, the method comprising:
monitoring over time a particle characteristic of air having particles generated by a particle generator in the indoor environment;
monitoring over time a gas characteristic of air having gas generated by a gas generator in the indoor environment;
determining at least one air characteristic of the indoor environment based on both the particle characteristic and the gas characteristic, wherein the monitoring of the particle characteristic and the monitoring of the gas characteristic are time-coordinated.

2. The method of claim 1, wherein the at least one air characteristic of the indoor environment includes a mitigation equivalent air change rate (MEACH) for a portion of the particle mitigation systems based on both the particle characteristic and the gas characteristic.

3. The method of claim 1, comprising determining a clean air delivery rate (CADR) for the indoor environment overall based on both the particle characteristic and the gas characteristic.

4. The method of claim 1, comprising determining a clean air delivery rate (CADR) for the indoor environment overall based on combining the mitigation equivalent air change rate (MEACH) for all particle mitigation systems and the air change rate (ACH) for all ventilation.

5. The method of claim 1, wherein the gas comprises CO2.

6. The method of claim 1, wherein the gas comprises isopropanol.

7. The method of claim 1, wherein the amount of gas provided to the indoor environment is a known, measured, or metered amount of gas.

8. The method of claim 1, wherein the gas is generated from a container.

9. The method of claim 1, wherein the gas is generated from one or more people present in the indoor environment.

10. The method of claim 1, comprising determining the at least one air characteristic of the indoor environment based on utilizing occupant number, occupant activity, or both, together with an expected CO2 production rate and a measured gas decay rate determined from recording gas concentration in the indoor environment over time.

11. The method of claim 1, comprising using a sensor to determine occupancy or occupant activity in the indoor environment, and occupants or occupant activity in the indoor environment generate the gas and charge the indoor environment.

12. The method of claim 1, comprising passively monitoring the particle characteristic of the air in the indoor environment to identify a background, a signal-to-background threshold, or both.

13. The method of claim 1, comprising passively monitoring the gas characteristic of the air in the indoor environment to identify: initiation of a gas generating event, termination of a gas generation event, achievement of a gas steady state, or any combination thereof.

14. The method of claim 1, comprising providing a known, measured, or metered amount of gas to the air of the indoor environment and determining a volume of the air of the indoor environment by utilizing a measured concentration of the gas with the known, measured, or metered amount of gas provided.

15. The method of claim 1, comprising assessing at least one air characteristic of the indoor environment against a air characteristic criteria; and altering a setting of one or more portion of the plurality of particle mitigation systems, a mechanical air filtration system, an air handling system, providing an alert, or any combination thereof.

16. The method of claim 1, wherein the particle generator is an occupant or occupant activity.

17. The method of claim 1, wherein the gas generator is one or more occupants in the indoor environment.

18. A system comprising a particle monitor, a gas monitor, a means for gas generation, a means for particle generation, and a control system, configured for performing the method of claim 1.

19. A system for analyzing air of an indoor environment having a plurality of particle mitigation systems, the system comprising:
a particle monitor configured to receive air having particles generated by a particle generator in the indoor environment and monitor a particle characteristic over time;
a gas monitor configured to receive air having a gas generated by a gas generator in the indoor environment and monitor a gas characteristic over time; and
a control system operatively coupled to the particle monitor and the gas monitor to determine at least one air characteristic of the indoor environment based on both the particle characteristic and the gas characteristic.

20. A system for analyzing air of an indoor environment having a plurality of particle mitigation systems including at least a mechanical air filtration system having a particle filtration profile wherein a first particle size is filtered less than a second particle size and an air handling system for exchanging air with an outdoor environment, the system comprising:
a particle generator configured to charge the indoor environment with a first charge including a concentration of a plurality of test particles, the plurality of test particles having a test particle size about equal to the first particle size;
a particle monitor configured to receive air from the indoor environment to detect the plurality of test particles;
a gas generator configured to charge the indoor environment with a second charge including a concentration of a test gas, the test gas having a size less than the test particle size;
a gas monitor configured to receive air from the indoor environment to detect the test gas; and
a control system operatively coupled to the particle monitor and the gas monitor to determine at least one air characteristic of the indoor environment based on a test gas characteristic related to a first portion of the particle mitigation systems of the indoor environment and a test particle characteristic related to a second portion of the particle mitigation systems of the indoor environment.
